(19) United States
(12) Patent Application Publication
(10) Pub. No.: US 2009/0203038 A1
(43) Pub. Date: Aug. 13, 2009

(54) Negative Mimic Antibody For Use as a Blocking Reagent In BNP Immunoassays

(75) Inventors: Susan E. Brophy, Lindenhurst, IL (US); William E. Brown, III, Libertyville, IL (US); Walt J. Keirans, Libertyville, IL (US); Joan D. Tyner, Beach Park, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/342,752

(22) Filed: Dec. 23, 2008

Related U.S. Application Data
(60) Provisional application No. 61/016,852, filed on Dec. 27, 2007.

(51) Int. Cl. G01N 33/53; C07K 16/00; C07H 21/04; C12N 5/06

(52) U.S. Cl. 435/7.1; 530/387.1; 530/387.3; 530/388.1; 530/389.1; 536/23.53; 435/358

(57) ABSTRACT

The present disclosure relates to isolated antibodies that do not bind to brain natriuretic peptide that can be used as a reagent to reduce heterophilic interference in an immunoassay.

### RELATED APPLICATION INFORMATION

This application claims the benefit of U.S. Application No. 61/016,852 filed on Dec. 27, 2008, the contents of which are herein incorporated by reference.

### TECHNICAL FIELD

The present disclosure relates to isolated antibodies that do not bind to brain natriuretic peptide. The antibodies of the present disclosure can be used as reagents to reduce or remove interference in immunoassays for brain natriuretic peptide or a fragment thereof.

### BACKGROUND

In the field of diagnostics, immunoassays are used for detecting analytes in biological samples. Examples of analytes that can be detected include, drugs, hormones, infectious agents, microorganisms, antibodies and the like. The identification of one or more analytes in a biological sample can be used to diagnose cancer, heart disease, etc.

Immunoassays involve a specific binding reaction between the analyte to be detected and at least one specific binding partner. The specific binding partner (which can be an antibody, antigen, etc) specifically binds to the analyte or reacts with it. The analyte and the specific binding partner form a specific binding pair complex. An example of such a specific binding pair complex is an antibody (or antibody fragment) and antigen. However, more than one analyte or more than one specific binding partner can react with each other during each reaction.

The specific binding pair complex can then be detected. Typically, at least one specific binding partner is labeled with a detectable label such as a chromogen, fluorophore, substances capable of chemi- or electrochemiluminescence, radioisotopes, haptens, enzymes labels or substances that can form another specific binding pair such as biotin and streptavidin.

As useful as immunoassays are, they are not without their problems. Such problems include nonspecific binding reactions and undesired interactions between the specific binding partners and the sample components. These interferences can cause an increase in background signal and reduced sensitivity and specificity. Depending on the kind of interference, false positive or false negative test results can occur.

When human serum/plasma is used as a biological sample in an immunoassay, heterophile or heterophilic antibodies present in a sample can interact with the specific binding partners and have an effect on the immunoassay. The existence of heterophilic antibodies and their potential for causing interference in immunoassays has been known for years. “Hetero” and “phile” are from the Greek and mean “different” and “affinity”, respectively. Taber's Medical Dictionary defines heterophilic antibodies as an “antibody response to an antigen other than a specific one” (See, Kaplan, I., et al., Clinical Chem., 45(5):616-618 (1999)). Heterophile antibodies are found in all people and are weak, multi-specific antibodies. There are a variety of possible causes for inducing heterophilic antibodies, including, exposure to animals, alternate animal contact therapy, exposure to animal products, special diets, deliberate immunization, rheumatoid factors, blood transfusions, autoimmune diseases, dialysis, maternal transfer, cardiac myopathy and gastrointestinal disease. It is apparent that heterophilic antibodies can come from natural antibodies (namely, natural polyspecific antibodies, natural idiotypic antibodies and natural rheumatoid factors (RF)) and autoantibodies (namely, autoimmune polyspecific antibodies and autoimmune RF and idiotypic antibodies). Heterophilic antibodies which bind to animal antibodies are referred to as HAAAs (human anti-animal antibodies). An example of an HAAA is a human anti-mouse antibody, also referred to as a HAMA.

A number of approaches are known in the art for reducing heterophilic antibody interference. Specifically, a number of physical and chemical techniques have been used to remove heterophile interferences. These techniques include, ultracentrifugation, removal of the interference with protein A or protein G, precipitation with trichloroacetic acid, heating at 90° C., and pretreatment with ethanol, polyethylene glycol (130 g/L), sulfhydryl agents and detergents (See, Levinson, S., et al., Clinica Chimica Acta, 325:1-15 (2002)). Alternatively, blocking agents can be used. A blocking reagent is a preparation, which, when added to an immunoassay, prevents interference by heterophilic antibodies. Two types of blocking agents are known, nonspecific blocking agents and specific blocking agents.

Historically, nonspecific blocking agents have been nonimmune globulins from the species used to produce the test antibodies. Id. The nonimmune globulin may be in the purified form, but usually, it is in the form of normal animal sera. Id. These agents have the advantage of containing a diverse mixture of proteins that can adsorb out heterophile interference from all types of sandwich and competitive assays. Id. Nonspecific binding agents are typically considered to be “passive” blocking agents because they are added in excess concentrations so that any specific anti-species antibodies present in the biological sample bind to these in preference to the specific binding partners that are present in lower concentrations.

Specific blocking agents are reagents that contain specific antibodies with activity against human immunoglobulins. Examples of commercially available specific blocking agents include immunoglobulin inhibiting reagent (IIR) and heterophile blocking reagent (HBR). IIR is a mixture of mouse monoclonal antibodies produced by using human heterophilic antibodies and HAMA as the immunogens. Id. IIR has been studied as an agent capable of blocking specific HAMA and it blocked interference in more samples than a polymerized monoclonal preparation, but not better than in a polyclonal nonimmune mouse IgG. Id.

Heterophile blocking reagents (HBR) are specific binders that are directed against the human heterophilic antibody. When HBR binds to the human heterophilic antibody, the blocking is accomplished by steric hindrance. One study suggested that HBR reduced heterophile interference from 29% to 4.8% compared with 1% normal mouse sera which reduced interference to 6.2% when an intact mouse monoclonal antibody was used for detection, while interference was reduced from 4.8% to 2.0% compared to 2.6% for the normal mouse sera when a chimeric detection antibody was used. Id. In another study, one patient's sera showed a reduction of troponin I to normal levels when HBR was added, but also to normal levels, when diluted with a preparation of normal goat serum. Id. In yet another study, HBR blocked interference in all of five sera that were previously positive prior to a modification of the reagent, but the manufacturer's modified reagent, containing bovine and goat normal sera, blocked the interference as well or better. Id. However, like nonspecific binding agents, specific binding agents do not remove interference in all cases.

Thereupon, there is a need in the art for new reagents that can be used to remove interferences from immunoassays.

### SUMMARY

In one embodiment, the present disclosure relates to an isolated antibody that does not bind to BNP. The antibody has a variable heavy domain and a variable light domain, the variable heavy domain comprising a heavy chain complementarity determining region (CDR) 1, a heavy chain CDR 2, a heavy chain CDR 3, a heavy framework region 1, a heavy framework region 2, a heavy framework region 3, a heavy framework region 4, the variable light domain comprising a light chain CDR 1, a light chain CDR 2, a light chain CDR 3, a light framework region 1, a light framework region 2, a light framework region 3, and a light framework region 4 wherein said antibody comprises at least one of:

(a) a heavy chain CDR 1 having an amino acid sequence of the formula of: a heavy chain CDR 1 having an amino acid sequence of the formula of:

(SEQ ID NO:1)

Gly-Tyr-Xaa₉-Phe-Thr-Xaa₁₀-Xaa₁₁-Xaa₁₂-Xaa₁₃-Xaa₁₄

wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine, provided that if (a) Xaa₉ is threonine, Xaa₁₀ is serine, Xaa₁₁ is tyrosine, Xaa₁₂ is tryptophan, Xaa₁₃ is methionine then Xaa₁₄ is glycine; (b) Xaa₁₀ is serine, Xaa₁₁ is tyrosine, Xaa₁₂ is tryptophan, Xaa₁₃ is methionine, Xaa₁₄ is alanine, then Xaa₉ is alanine; (c) Xaa₉ is threonine, Xaa₁₁ is tyrosine, Xaa₁₂ is tryptophan, Xaa₁₃ is methionine, Xaa₁₄ is asparagine, then Xaa₁₀ is alanine; (d) Xaa₉ is threonine, Xaa₁₀ is serine, Xaa₁₂ is tryptophan, Xaa₁₃ is methionine, Xaa₁₄ is asparagine, then Xaa₁₁ is aspartic acid or proline; (e) Xaa₉ is threonine, Xaa₁₀ is serine, Xaa₁₁ is tyrosine, Xaa₁₃ is methionine, Xaa₁₄ is asparagine then Xaa₁₂ is cysteine, glutamine, proline, valine or asparagine; or (f) Xaa₉ is threonine, Xaa₁₀ is serine, Xaa₁₁ is tyrosine, Xaa₁₂ is tryptophan, Xaa₁₄ is asparagine then Xaa₁₃ is proline, threonine, lysine or histidine;

(b) a heavy chain CDR 2 having an amino acid sequence of the formula of:

(SEQ ID NO:2)

Arg-Ile-Xaa₁-Xaa₂-Xaa₃-Xaa₄-Xaa₅-Glu-Thr-His-Tyr-
Asn-Gln-Lys-Phe-Lys-Asp

wherein Xaa₁ is aspartic acid or cysteine; Xaa₂ is proline or leucine; Xaa₃ is tyrosine, proline or methionine; Xaa₄ is aspartic acid or serine and Xaa₅ is serine or leucine, provided that if: (a) Xaa₁ is aspartic acid, Xaa₂ is proline, Xaa₃ is tyrosine, Xaa₄ is aspartic acid then Xaa₅ is leucine; (b) Xaa₂ is proline, Xaa₃ is tyrosine, Xaa₃ is aspartic acid, Xaa₅ is serine, then Xaa₁ is cysteine; (c) Xaa₁ is aspartic acid, Xaa₃ is tyrosine, Xaa₄ is aspartic acid, Xaa₅ is serine, then Xaa₂ is leucine; or (d) Xaa₁ is aspartic acid, Xaa₂ is proline, Xaa₄ is aspartic acid, Xaa₅ is serine, then Xaa₃ is proline or methionine; or (e) Xaa₁ is aspartic acid, Xaa₂ is proline, Xaa₃ is tyrosine, Xaa₅ is serine, then Xaa₄ is serine;

(c) a light chain CDR 1 that has an amino acid sequence having a formula of:

(SEQ ID NO:4)

Xaa₁₅-Xaa₁₆-Xaa₁₇-Xaa₁₈-Xaa₁₉-Xaa₂₀-Leu-Asp-Ser-
Asp-Gly-Xaa₂₁-Xaa₂₂-Xaa₂₃-Leu-Asn

wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline, provided that if: (a) Xaa₁₅ is lysine; Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, then Xaa₂₃ is proline; (b) Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₅ is leucine; (c) Xaa₁₅ is lysine, Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₆ is valine or leucine; (d) Xaa₁₅ is lysine, Xaa₁₆ is serine; Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₇ is arginine, aspartic acid or threonine; (e) Xaa₁₅ is lysine, Xaa₁₆ is serine; Xaa₁₇ is serine, Xaa₁₉ is serine, Xaa₂₀ is leucine, Xaa₂₁ is lysine, Xaa₂₂ is threonine, Xaa₂₃ is tyrosine, then Xaa₁₈ is glutamic acid; (f) Xaa₁₅ is lysine, Xaa₁₆ is serine; Xaa₁₇ is serine, Xaa₁₈ is glutamine, Xaa₂₀ is leucine, Xaa₂₁ is lysine, Xaa₂₂ is threonine, Xaa₂₃ is tyrosine, then Xaa₁₉ is aspartic acid or leucine; (g) Xaa₁₅ is lysine, Xaa₁₆ is serine; Xaa₁₇ is serine, Xaa₁₈ is glutamine, Xaa₁₉ is serine, Xaa₂₁ is lysine, Xaa₂₂ is threonine, Xaa₂₃ is tyrosine, then Xaa₂₀ is glutamine; (h) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₂ is threonine, Xaa₂₃ is tyrosine, then Xaa₂₁ is glutamine; or (i) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₁ is lysine, Xaa₂₃ is tyrosine, then Xaa₂₂ is glycine;

(d) a light chain CDR 2 that has an amino acid sequence having a formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine, provided that if (a) Xaa₆ is serine and Xaa₇ is lysine, then Xaa₈ is glutamine; (b) Xaa₇ is lysine and Xaa₈ is leucine, then Xaa₆ is glutamic acid; or (c) Xaa₆ is serine and Xaa₈ is leucine, then Xaa₇ is proline;

(e) a heavy chain framework 1 that has an amino acid sequence having a formula of:

(SEQ ID NO:114)

Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-Ala-
Ser

wherein Xaa₂₄ is alanine;

(f) a heavy chain framework 2 that has an amino acid sequence having a formula of:

(SEQ ID NO:116)

Trp-Val-Lys-Gln-Arg-Pro-Glu-Gln-Gly-Leu-Glu-Trp-
Ile-Xaa₂₅

wherein Xaa₂₅ glutamic acid;

(g) a light chain framework 2 that has an amino acid sequence having a formula of:

(SEQ ID NO:121)

Trp-Xaa₂₆-Phe-Gln-Xaa₂₇-Pro-Gly-Glu-Ser-Pro-Lys-
Leu-Leu-Ile-Tyr

wherein Xaa₂₆ is leucine or serine and Xaa₂₇ is arginine or glycine, provided that if (a) Xaa₂₆ is leucine, then Xaa₂₇ is glycine; or (b) Xaa₂₇ is arginine, then Xaa₂₆ is serine;

(h) a light chain framework 4 that has an amino acid sequence having a formula of:

(SEQ ID NO:125)

Trp-Thr-Phe-Gly-Gly-Gly-Thr-Lys-Leu-Glu-Ile-Lys-
Xaa₂₈

wherein Xaa₂₈ is tryptophan; or

(i) any combinations of (a)-(h).

The above antibody can be a monoclonal antibody, a multispecific antibody, a human antibody, a fully humanized antibody, a partially humanized antibody, an animal antibody, a recombinant antibody, a chimeric antibody, a single-chain Fv, a single chain antibody, a single domain antibody, a Fab fragment, a F(ab′)₂ fragment, a disulfide-linked Fv, a DVD-Ig, a TVD-Ig, an anti-idiotypic antibody, or a functionally active epitope-binding fragment thereof.

In yet another embodiment, the present disclosure relates to an isolated antibody that does not bind to BNP. The antibody has a variable heavy domain and a variable light domain, the variable heavy domain comprising a heavy chain complementarity determining region (CDR) 1, a heavy chain CDR 2, a heavy chain CDR 3, a heavy framework region 1, a heavy framework region 2, a heavy framework region 3, a heavy framework region 4, the variable light domain comprising a light chain CDR 1, a light chain CDR 2, a light chain CDR 3, a light framework region 1, a light framework region 2, a light framework region 3, and a light framework region 4 wherein said antibody comprises at least one of:

(a) the heavy chain CDR 1 has an amino acid sequence having a formula of:

(SEQ ID NO:1)

Gly-Tyr-Xaa₉-Phe-Thr-Xaa₁₀-Xaa₁₁-Xaa₁₂-Xaa₁₃-Xaa₁₄

wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine;

(b) the heavy chain CDR 2 has an amino acid sequence having a formula of:

(SEQ ID NO:2)

Arg-Ile-Xaa₁-Xaa₂-Xaa₃-Xaa₄-Xaa₅-Glu-Thr-His-Tyr-
Asn-Gln-Lys-Phe-Lys-Asp

wherein Xaa₁ is aspartic acid or cysteine; Xaa₂ is proline or leucine; Xaa₃ is tyrosine, proline or methionine; Xaa₄ is aspartic acid or serine and Xaa₅ is serine or leucine;

(c) the heavy chain CDR 3 has an amino acid sequence of:

| | |
|---|---|
| Asp-Gly-Tyr; | (SEQ ID NO:3) |

(d) the light chain CDR 1 has an amino acid sequence having a formula of:

(SEQ ID NO:4)

Xaa₁₅-Xaa₁₆-Xaa₁₇-Xaa₁₈-Xaa₁₉-Xaa₂₀-Leu-Asp-Ser-
Asp-Gly-Xaa₂₁-Xaa₂₂-Xaa₂₃-Leu-Asn

wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline;

(e) the light chain CDR 2 has an amino acid sequence having the formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine;

(f) the light chain CDR 3 has an amino acid sequence having a formula of:

| | |
|---|---|
| Leu-Gln-Ala-Thr-His-Phe-Pro; | (SEQ ID NO:6) |

(g) the heavy chain framework 1 has an amino acid sequence having a formula of:

(SEQ ID NO:130)

Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-Ala-
Ser

wherein Xaa₂₄ is valine or alanine;

(h) the heavy chain framework 2 has an amino acid sequence having a formula of:

(SEQ ID NO:131)

Trp-Val-Lys-Gln-Arg-Pro-Glu-Gln-Gly-Leu-Glu-Trp-
Ile-Xaa₂₅

wherein Xaa₂₅ is glycine or glutamic acid;

(i) the heavy chain framework 3 has an amino acid sequence having a formula of:

(SEQ ID NO:118)

Lys-Ala-Ile-Leu-Thr-Val-Asp-Lys-Ser-Ser-Ser-Thr-
Ala-Phe-Val-Gln-Leu-Thr-Ser-Leu-Thr-Ser-Glu-Asp-
Ser-Ala-Val-Tyr-Tyr-Cys-Val-Ser;

(j) the heavy chain framework 4 has an amino acid sequence having a formula of:

(SEQ ID NO:119)

Trp-Gly-Ala-Gly-Thr-Thr-Cys-Thr-Val-Ser-Ser;

(k) the light chain framework 1 has an amino acid sequence having a formula of:

(SEQ ID NO:120)

Asp-Val-Val-Met-Thr-Gln-Thr-Pro-Leu-Thr-Leu-Ser-
Val-Thr-Thr-Gly-Gln-Pro-Ala-Ser-Ile-Ser-Cys;

(l) the light chain framework 2 has an amino acid sequence having a formula of:

(SEQ ID NO:121)

Trp-Xaa₂₆-Phe-Gln-Xaa₂₇-Pro-Gly-Glu-Ser-Pro-Lys-
Leu-Leu-Ile-Tyr

wherein Xaa₂₆ is leucine or serine;

wherein Xaa₂₇ is arginine or glycine;

(m) the light chain framework 3 has an amino acid sequence having a formula of:

(SEQ ID NO:124)

Gly-Val-Pro-Asp-Arg-Phe-Thr-Gly-Ser-Gly-Ser-Gly-
Thr-Asp-Phe-Thr-Leu-Lys-Ile-Ser-Arg-Val-Glu-Ala-
Glu-Asp-leu-Gly-Val-Tyr-Tyr-Cys;
and

(n) the light chain framework 4 has an amino acid sequence having a formula of:

(SEQ ID NO:132)

Trp-Thr-Phe-Gly-Gly-Gly-Thr-Lys-Leu-Glu-Ile-Lys-
Xaa₂₈

wherein Xaa₂₈ is arginine or tryptophan;

provided that each of Xaa₁-Xaa₂₈ are not simultaneously the following: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg.

In the above antibody, Xaa₁-Xaa₂₈ can be as follows: (a) Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ala, Xaa₁₁ is Tyr, Xaa₁₂ is Cys, Xaa₁₃ is Met, Xaa₁₄ is Asn, Xaa₁₅ is Lys, Xaa₁₆ is Val, Xaa₁₇ is Arg, Xaa₁₈ is Glu, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg; (b) Xaa₁ is Cys, Xaa₂ is Leu, Xaa₃ is Met, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Gln, Xaa₁₃ is Pro, Xaa₁₄ is Gly, Xaa₁₅ is Lys, Xaa₁₆ is Ser, Xaa₁₇ is Asp, Xaa₁₈ is Leu, Xaa₁₉ is Asp, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Ser, Xaa₂₇ is Arg and Xaa₂₈ is Arg; (c) Xaa₁ is Asp, Xaa₂ is Pro, Xaa₃ is Pro, Xaa₄ is Ser, Xaa₅ is Leu, Xaa₆ is Glu, Xaa₇ is Pro, Xaa₈ is Gln, Xaa₉ is Thr, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg; (d) Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Pro, Xaa₁₃ is Thr, Xaa₁₄ is Gly, Xaa₁₅ is Leu, Xaa₁₆ is Leu, Xaa₁₇ is Thr, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Glu, Xaa₂₆ is Leu, Xaa₂₇ is Gly and Xaa₂₈ is Arg; (e) Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Asp, Xaa₁₂ is Val, Xaa₁₃ is Lys, Xaa₁₄ is Asn, Xaa₁₅ is Lys, Xaa₁₆ is Ser, Xaa₁₇ is Ser, Xaa₁₈ is Ser, Xaa₁₉ is Leu, Xaa₂₀ is Gln, Xaa₂₁ is Lys, Xaa₂₂ is Thr, Xaa₂₃ is Tyr, Xaa₂₄ is Ala, Xaa₂₅ is Gly, Xaa₂₆ is Leu, Xaa₂₇ is Arg and Xaa₂₈ is Arg; (f) Xaa₁ is Asp, Xaa₂ is Pro, Xaa₃ is Pro, Xaa₄ is Ser, Xaa₅ is Leu, Xaa₆ is Ser, Xaa₇ is Lys, Xaa₈ is Leu, Xaa₉ is Ala, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg; or (g) Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Pro, Xaa₁₂ is Asn, Xaa₁₃ is His, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Gln, Xaa₂₂ is Gly, Xaa₂₃ is Pro, Xaa₂₄ is Val, Xaa₂₅ is Gly, Xaa₂₆ is Leu, Xaa₂₇ is Arg and Xaa₂₈ is Trp.

In the above antibody, (a) the heavy chain CDR 1 has an amino acid sequence comprising SEQ ID NO:102, SEQ ID NO:103, SEQ ID NO:104, SEQ ID NO:105, SEQ ID NO:106 or SEQ ID NO:107; (b) the heavy chain CDR 2 has an amino acid sequence comprising SEQ ID NO:69, SEQ ID NO:108 or SEQ ID NO:129; (c) the light chain CDR 1 has an amino acid sequence comprising SEQ ID NO:109, SEQ ID NO: 110, SEQ ID NO:111, SEQ ID NO:112 or SEQ ID NO:113; (d) the light chain CDR 2 has an amino acid sequence comprising SEQ ID NO:70; (e) the heavy chain framework 1 has an amino acid sequence comprising SEQ ID NO:115; (f) the heavy chain framework 2 has an amino acid sequence comprising SEQ ID NO:117; (g) the light chain framework 2 has an amino acid sequence comprising SEQ ID NO:122 or SEQ ID NO:123; or (h) the light chain framework 4 has an amino acid sequence comprising SEQ ID NO:126.

More specifically, in the above antibody, (a) the heavy chain CDR 2 has an amino acid sequence comprising SEQ ID NO:69; and (b) the light chain CDR 2 has an amino acid sequence comprising SEQ ID NO:70.

The above antibody can be a monoclonal antibody, a multispecific antibody, a human antibody, a fully humanized antibody, a partially humanized antibody, an animal antibody, a recombinant antibody, a chimeric antibody, a single-chain Fv, a single chain antibody, a single domain antibody, a Fab fragment, a F(ab′)₂ fragment, a disulfide-linked Fv, a DVD-Ig, a TVD-Ig, an anti-idiotypic antibody, or a functionally active epitope-binding fragment thereof.

In still yet another embodiment, the present disclosure relates to an isolated polynucleotide comprising a polynucleotide sequence having a sequence of SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101 and full complements thereof.

In yet still another embodiment, the present disclosure relates to a heterophilic blocking agent for use in a BNP immunoassay, the agent comprising any of the above described antibodies.

In still yet another embodiment, the present disclosure relates to a method for reducing heterophilic interference in a BNP immunoassay. The method comprises the step of adding the above described heterophilic blocking agent to an immunoassay.

In still yet another embodiment, the present disclosure relates to an immunoassay for hBNP or hBNP fragment, wherein said immunoassay comprises at least one of the above described antibodies.

In still yet another embodiment, the present disclosure relates to chinese hamster ovary (“CHO”) cell line BNP3-631-436CN4CHO that expresses the CN4 antibody, wherein said cell line comprises at least one polynucleotide sequence selected from the group consisting of: SEQ ID NO:82, SEQ ID NO:83, SEQ ID NO:84, SEQ ID NO:85 and combinations thereof.

In still yet another embodiment, the present disclosure relates to an antibody made from DNA extracted from the CHO cell line BNP3-631-436CN4CHO, wherein said DNA comprises at least one polynucleotide sequence selected from the group consisting of: SEQ ID NO:82, SEQ ID NO:83, SEQ ID NO:84, SEQ ID NO:85 and combinations thereof.

In still yet another embodiment, the present disclosure relates to a chimeric antibody or a fragment thereof produced by CHO cell line BNP3-631-436CN4CHO, wherein said antibody comprises DNA comprising at least one polynucleotide sequence selected from the group consisting of: SEQ ID NO:82, SEQ ID NO:83, SEQ ID NO:84, SEQ ID NO:85 and combinations thereof.

In still yet another embodiment, the present disclosure relates to a kit for use in an immunoassay for hBNP or hBNP fragment. The kit comprises:
(a) heterophilic blocking agent comprising at least one of the above described antibodies; and
(b) instructions for conducting the assay of the test sample.

### BRIEF DESCRIPTION OF THE FIGURES

FIG. 1 is a diagram of the 3-631-436 scFv protein in the orientation in which it is expressed on the surface of yeast.

FIG. 2 shows the amino acid sequence of the 3-631-436 scFv (SEQ ID NO: 66). The solid underlined sequence represents the variable heavy chain sequence (VH), the double underlined sequence the linker, and the stippled underline sequence the variable light chain sequence (VL). Italicized and bold type indicates the complementary determining regions (CDR).

FIGS. 3A-3B show the nucleotide sequence of the 3-631-436 scFv (SEQ ID NO: 67). The double-stranded sequence is depicted.

FIG. 4 is a plasmid map for vector pYD41-40-3631436-scFv containing the 3-631-436 single-chain variable fragment shown in FIG. 1.

FIGS. 5A-5B show that yeast expressing full-length 3-631-436 scFv bind to cyclic BNP (SEQ ID NO: 10). More specifically, this figure shows that 3-631-436 scFv expressing yeast were incubated with cyclic BNP (1-32c) (SEQ ID NO:10) or anti-V5 followed by secondary reagents streptavidin phycoerythrin (SA:PE) (FIG. 5A) and goat anti mouse-FITC (GAM:FITC) (FIG. 5B). The flow cytometry histograms illustrate the full-length expression of 3-631-436 scFv as detected by anti-V5 (5B) and the ability of 3-631-436 scFv to bind to the antigen cyclic BNP peptide (1-32) (SEQ ID NO: 5) (5A). Antigen (Ag) binding or PE-A units (abscissa): 10⁰, 10¹, 10², and 10³ and 10⁴. Count units (ordinate): 0, 10, 20, 30, 40, 50, 60, 70, 80 (FIG. 5A); Antibody (Ab) expression or FITC-A units (abscissa) 10⁰, 10¹, 10², and 10³ and 10⁴: 0, 10, 20, 30, 40, 50, 60, 70, 80 (FIG. 5B).

FIGS. 6A-6D are the nucleotide sequence of the vector shown in FIG. 4 (SEQ ID NO: 11).

FIG. 7 is a schematic depiction which shows how degenerate oligonucleotides were designed so that three amino acid positions of the complementarity determining region (9 nucleotides) were randomly mutated per library.

FIG. 8 is a schematic depiction which shows how the 3-631-436 scFv library was constructed using yeast homologous recombination. More specifically, gapped vectors were PCR generated to exclude those nucleotides that were being mutagenized in the library. The degenerate single stranded oligonucleotides were synthesized. Gapped vectors and single stranded degenerate oligonucleotides were transformed into S. cerevisiae strain EBY100. Transformed clones were selected in tryptophan deficient glucose media.

FIGS. 9A-9F show the fifty (50) oligonucleotides (SEQ ID NOS:14-63) that were used to create the pYD1-41-40-3-631-436 vectors described in detail in Example 1.

FIG. 10 are graphs that show 3-631-436 WT scFv, 3-631-436 CDR light chain libraries (3631VL) and CDR heavy chain libraries (3631 VH) were incubated with or without biotinylated:cyclic BNP (1-32c) (SEQ ID NO:10) and anti-V5 followed by secondary reagents streptavidin phycoerythrin (SA:PE) and goat anti mouse-FITC (GAM:FITC). Using FACS, a sort gate (“S1” or “S2”) was set in which 3-631-436 scFv yeast cells that did not bind BNP (PE low fluorescence) but were expressed as full length proteins (FITC high fluorescence) were sorted. Selected yeast cells were grown, stained and sorted a second time as described above. Finally, round 2 sorted cells were plated on selective media and further characterized. The “pos” or positive control gate was set based on the position of the 3-631-436WT BNP binding population.

FIG. 11 shows the results of the characterization of scFv clones and wildtype (WT) clones from 3-631-436 for human brain natriuretic peptide (BNP) and anti-V5 binding pursuant to Example 1. The characterization shows that clones H1, H3, H4, L1, L2, L3 and L4 did not bind human BNP at 83 nM.

FIG. 12 shows the amino acid mutations for the 3-631-436 scFv variants.

FIG. 13 shows a 3-631-436 VH/VL combination library was incubated with or without biotinylated:cyclic BNP (1-32c) (SEQ ID NO:10) and anti-V5 followed by secondary reagents streptavidin phycoerythrin (SA:PE) and goat anti mouse-FITC (GAM:FITC). Using FACS, a sort gate (“S1” and “S2”) was set in which 3-631-436 scFv yeast cells that did not bind BNP (PE low fluorescence) but were expressed as full length proteins (FITC high fluorescence) were sorted. Selected yeast cells were grown, stained and sorted a second time from “S2” gate. Finally, round 2 sorted cells were plated on selective media and further characterized. The “WT pos” or positive control gate was set based on the position of the 3-631-436WT BNP binding population.

FIG. 14 shows the further characterization of clones CN1, CN2, CN4, CN6, CN7, CN8, CN9 and WT described in Example 1 at 2 μM human BNP.

FIG. 15 shows the amino acid mutations in the CDR heavy chain and CDR light chain for each of clones CN1, CN2, CN4, CN6, CN7, CN8 and CN9.

FIG. 16 shows KinExA® characterization of the binding of clone CN4, to human BNP. Clone CN4 did not generate a signal at both 1 nM and 10 nM monoclonal antibody (mAb) concentration demonstrating its lack of ability to bind to human BNP. Voltage values are recorded in the table to illustrate the lack of binding to BNP relative to the positive control (3-631-436 WT mAb).

FIG. 17 shows polynucleotide sequences for each of the CDR heavy chain and CDR light chain for each of clones CN1, CN2, CN4, CN6, CN7, CN8 and CN9 Specifically, FIG. 17A shows the polynucleotide sequence of the CDR heavy chain for clone CN1 (SEQ ID NOS:74 and 75). FIG. 17B shows the polynucleotide sequence of the CDR light chain for clone CN1 (SEQ ID NOS:76 and 77). FIG. 17C shows the polynucleotide sequence of the CDR heavy chain for clone CN2 (SEQ ID NOS:78 and 79). FIG. 17D shows the polynucleotide sequence of the CDR light chain for clone CN2 (SEQ ID NOS:80 and 81). FIG. 17E shows the polynucleotide sequence of the CDR heavy chain for clone CN4 (SEQ ID NOS:82 and 83). FIG. 17F shows the polynucleotide sequence of the CDR light chain for clone CN4 (SEQ ID NOS:84 and 85). FIG. 17G shows the polynucleotide sequence of the CDR heavy chain for clone CN6 (SEQ ID NOS:86 and 87). FIG. 17H shows the polynucleotide sequence of the CDR light chain for clone CN6 (SEQ ID NOS:88 and 89). FIG. 17I shows the polynucleotide sequence of the CDR heavy chain for clone CN7 (SEQ ID NOS:90 and 91). FIG. 17J shows the polynucleotide sequence of the CDR light chain for clone CN7 (SEQ ID NOS:92 and 93). FIG. 17K shows the polynucleotide sequence of the CDR heavy chain for clone CN8 (SEQ ID NOS:94 and 95). FIG. 17L shows the polynucleotide sequence of the CDR light chain for clone CN8 (SEQ ID NOS:96 and 97). FIG. 17M shows the polynucleotide sequence of the CDR heavy chain for clone CN9 (SEQ ID NOS:98 and 99). FIG. 17N shows the polynucleotide sequence of the CDR light chain for clone CN9 (SEQ ID NOS:100 and 101).

FIG. 18 shows the amino acid sequences for the variable heavy chain (SEQ ID NO:127) and variable light chain (SEQ ID NO:128) for wildtype 3-631-436. FIG. 18A shows the variable heavy chain, including framework H1 (amino acids 1-25), CDR1 (amino acids 26-35), framework H2 (amino acids 36-49), CDR2 (amino acids 50-65), framework H3 (amino acids 66-94), CDR3 (amino acids 95-97) and framework H4 (amino acids 103-113). FIG. 18B shows the variable light chain, including framework L1 (amino acids 1-23), CDR1 (amino acids 24-34), framework L2 (amino acids 35-49), CDR2 (amino acids 50-56), framework L3 (amino acids 57-88), CDR3 (amino acids 89-95) and framework L4 (amino acids 96-108).

### DETAILED DESCRIPTION

The present disclosure relates to negative mimic antibodies or NEMIC antibodies. The antibodies of the present disclosure can be used as heterophilic blocking agents in BNP immunoassays. Specifically, the antibodies of the present disclosure can be used to reduce heterophilic interference in said immunoassays.

### A. Definitions

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) Antibody or Antibodies

As used herein, the terms “antibody” and “antibodies” refer to monoclonal antibodies, multispecific antibodies, human antibodies, humanized antibodies (fully or partially humanized), animal antibodies (in one aspect, a bird (for example, a duck or goose), in another aspect, a shark or whale, in yet another aspect, a mammal, including a non-primate (for example, a cow, pig, camel, llama, horse, goat, rabbit, sheep, hamsters, guinea pig, cat, dog, rat, mouse, etc) and a non-human primate (for example, a monkey, such as a cynomologous monkey, a chimpanzee, etc), recombinant antibodies, chimeric antibodies, single-chain Fvs (scFv), single chain antibodies, single domain antibodies, dual variable domain (DVD) or triple variable domain (TVD) antibodies (Dual-variable domain immunoglobulins and methods for making them are described in Wu, C., et al., Nature Biotechnology, 25(11):1290-1297 (2007) and WO2001/058956, the contents of each of which are herein incorporated by reference), Fab fragments, F(ab′)₂ fragments, disulfide-linked Fv (sdFv), and anti-idiotypic (anti-Id) antibodies (including, for example, anti-Id antibodies to antibodies of the present disclosure), and functionally active epitope-binding fragments of any of the above. In particular, antibodies include immunoglobulin molecules and immunologically active fragments of immunoglobulin molecules, namely, molecules that contain an antigen binding site. Immunoglobulin molecules can be of any type (for example, IgG, IgE, IgM, IgD, IgA and IgY), class (for example, IgG₁, IgG₂, IgG₃, IgG₄, IgA₁ and IgA₂) or subclass.

b) Binding Constants

The term “association rate constant”, “kₒₙ” or “kₐ” as used interchangeably herein, refers to the value indicating the binding rate of an antibody to its target antigen or the rate of complex formation between an antibody and antigen as shown by the equation below:

Antibody (“Ab”)+Antigen (“Ag”)→Ab-Ag.

The term “dissociation rate constant”, “k_{off}” or “k_{d}” as used interchangeably herein, refers to the value indicating the dissociation rate of an antibody from its target antigen or separation of Ab-Ag complex over time into free antibody and antigen as shown by the equation below:

Ab+Ag←Ab-Ag.

Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.) can also be used.

As used herein, the term “equilibrium dissociation constant” or “K_{D}” as used interchangeably, herein, refers to the value obtained by dividing the dissociation rate (k_{off}) by the association rate (kₒₙ). The association rate, the dissociation rate and the equilibrium dissociation constant are used to represent the binding affinity of an antibody to an antigen.

c) Epitope or Epitopes

As used herein, the term “epitope” or “epitopes” refers to sites or fragments of a polypeptide or protein having antigenic or immunogenic activity in a subject. An epitope having immunogenic activity is a site or fragment of a polypeptide or protein that elicits an antibody response in an animal. An epitope having antigenic activity is a site or fragment of a polypeptide or protein to which an antibody immunospecifically binds as determined by any method well-known to those skilled in the art, for example by immunoassays.

d) Human Brain Natriuretic Peptide, Human BNP, hBNP, hBNP Peptide or B-Type Natriuretic Peptide

As used herein, the term “human brain natriuretic peptide”, “human BNP”, “hBNP”, “hBNP peptide”, “B-type natriuretic peptide”, “hBNP polypeptide” hBNP 1-32 refers to a 32 amino acid molecule representing amino acids 77-108 of the 108 amino acid precursor molecule of human brain natriuretic peptide. The sequence of human brain naturetic peptide is shown in SEQ ID NO: 68.

e) hBNP Fragment or hBNP Peptide Fragment

As used herein, the term “hBNP fragment” or “hBNP peptide fragment” as used herein refers to a polypeptide that comprises at least about five contiguous amino acids of amino acids 77-108 of the 108 amino acid BNP precursor molecule (See, SEQ ID NO: 68). In one aspect, a hBNP fragment or hBNP peptide fragment refers to a polypeptide that comprises at least about ten contiguous amino acids residues of amino acids 77-108 of the 108 amino acid BNP precursor molecule; at least about fifteen contiguous amino acids residues of amino acids 77-108 of the 108 amino acid BNP precursor molecule; at least about 20 contiguous amino acids residues of amino acids 77-108 of the 108 amino acid BNP precursor molecule; at least about 25 contiguous amino acids residues of amino acids 77-108 of the 108 amino acid BNP precursor molecule, or at least about 30 contiguous amino acid residues of amino acids 77-108 of the 108 amino acid BNP precursor molecule. Examples of hBNP fragments or hBNP peptide fragments include, but are not limited to, amino acid sequences containing amino acids residues 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 2-32, 2-31, 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 3-32, 3-31, 3-30, 3-29, 3-28, 3-27, 3-26, 3-25, 3-24, 3-23, 3-32, 3-21, 3-20, 3-19, 3-18, 3-17, 3-16, 3-15, 3-14, 3-13, 3-12, 3-11, 3-10, 3-9, 3-8, 4-32, 4-31, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 7-32, 7-31, 7-30, 7-29, 7-28, 7-27, 7-26, 7-25, 7-24, 7-23, 7-22, 7-21, 7-20, 7-19, 7-18, 7-17, 7-16, 7-15, 7-14, 7-13, 7-12, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 9-32, 9-31, 9-30, 9-29, 9-28, 9-27, 9-26, 9-25, 9-24, 9-23, 9-22, 9-21, 9-20, 9-19, 9-18, 9-17, 9-16, 9-15, 9-14, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 11-32, 11-31, 11-30, 11-29, 11-28, 11-27, 11-26, 11-25, 11-24, 11-23, 11-22, 11-21, 11-20, 11-19, 11-18, 11-17, 11-16, 12-32, 12-31, 12-30, 12-29, 12-28, 12-27, 12-26, 12-25, 12-24, 12-23, 12-22, 12-21, 12-20, 12-19, 12-18, 12-17, 13-32, 13-31, 13-30, 13-29, 13-28, 13-27, 13-26, 13-25, 13-24, 13-23, 13-22, 13-21, 13-20, 13-19, 13-18, 14-32, 14-31, 14-30, 14-29, 14-28, 14-27, 14-26, 14-25, 14-24, 14-23, 14-22, 14-21, 14-20, 14-19, 15-32, 15-31, 15-30, 15-29, 15-28, 15-27, 15-26, 15-25, 15-24, 15-23, 15-22, 15-21, 15-20, 16-32, 16-31, 16-30, 16-29, 16-28, 16-27, 16-26, 16-25, 16-24, 16-23, 16-22, 16-21, 17-32, 17-31, 17-30, 17-29, 17-28, 17-27, 17-26, 17-25, 17-24, 17-23, 17-22, 18-32, 18-31, 18-30, 18-29, 18-28, 18-27, 18-26, 18-25, 18-24, 18-23, 19-32, 19-31, 19-30, 19-29, 19-28, 19-27, 19-26, 19-25, 19-24, 20-32, 20-31, 20-30, 20-29, 20-28, 20-27, 20-26, 20-25, 21-32, 21-31, 21-30, 21-29, 21-28, 21-27, 21-26, 22-32, 22-31, 22-30, 22-29, 22-28, 22-27, 23-32, 23-31, 23-30, 23-29, 23-28, 24-32, 24-31, 24-30, 24-29, 25-32, 25-31, 25-20, 26-32, 26-31 or 27-32 of hBNP.

f) Humanized Antibody

As used herein, the term “humanized” antibody refers to an immunoglobulin variant or fragment thereof, which is capable of binding to a predetermined antigen and which comprises framework regions having substantially the amino acid sequence of a human immunoglobulin and CDRs having substantially the amino acid sequence of a non-human immunoglobulin. Ordinarily, a humanized antibody has one or more amino acid residues introduced into it from a source that is non-human. In general, the humanized antibody will include substantially all of at least one, and typically two, variable domains (Fab, Fab′, F(ab′)₂, Fabc, Fv) in which all or substantially all of the CDR regions correspond to those of a non-human immunoglobulin and all or substantially all of the FR regions are those of a human immunoglobulin consensus sequence. The humanized antibody optimally comprises at least a portion of an immunoglobulin constant region (Fc), typically that of a human immunoglobulin. Generally, the antibody will contain both the light chain as well as at least the variable domain of a heavy chain. The humanized antibody can be selected from any class of immunoglobulins, including IgM, IgG, IgD, IgA and IgE, and any isotype, including IgG₁, IgG₂, IgG₃ and IgG₄. The humanized antibody may comprise sequences from more than one class or isotype, and selecting particular constant domains to optimize desired effector functions is within those skilled in the art.

g) Immunospecifically Binds

As used herein, the phrase “immunospecifically binds to brain natriuretic peptide”, “immunospecifically binds to a brain natriuretic fragment”, “immunospecifically binds to human brain natriuretic peptide”, “immunospecifically binds to hBNP”, “immunospecifically binds to human brain natriuretic peptide fragment” or “immunospecifically binds to hBNP fragment” and analogous terms thereof refer to peptides, polypeptides, proteins, fusion proteins and antibodies that specifically bind to BNP, a BNP fragment, hBNP or a hBNP fragment and do not specifically bind to other peptides. A peptide, polypeptide, protein, or antibody that immunospecifically binds to BNP, a BNP fragment, hBNP or a hBNP fragment may bind to other peptides, polypeptides, or proteins with lower binding affinity as determined by, for example, immunoassays, such as a Biacore® or KinExA® assay, or any other assays known to those of skill in the art. Antibodies or antibody fragments that immunospecifically bind to BNP, a BNP fragment, hBNP or a hBNP fragment can be identified, for example, by immunoassays, such as by using a Biacore® or KinExA® assay, or by using other techniques known to those of skill in the art. An antibody binds immunospecifically to BNP, a BNP fragment, hBNP peptide or hBNP fragment when it binds to BNP, a BNP fragment, hBNP or a hBNP fragment with a higher binding affinity than to any cross-reactive antigen as determined using experimental techniques, such as, but not limited to, radioimmunoassays (RIA) and enzyme-linked immunosorbent assays (ELISAs) (See, for example, Paul, ed., Fundamental Immunology, 2nd ed., Raven Press, New York, pages 332-336 (1989) for a discussion regarding antibody specificity.).

h) Isolated

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In one aspect, nucleic acid molecules are isolated. In another aspect, a nucleic acid molecule encoding an antibody of the disclosure is isolated.

i) Negative Mimic or NEMIC Antibody

As used herein, the term “negative mimic” antibody or “NEMIC” antibody refers to an antibody that does not immunospecifically bind to an antigen of interest (such as, but not limited to a protein, peptide, fragment of a protein or peptide, etc.). In other words, the NEMIC antibody of the present disclosure does not generate any detectable signal when placed in the presence of the antigen of interest under standard assay conditions. Such assay conditions are well known in the art and can include the use of a Biacore® or KinExA® assay (See, for example, the use of a KinExA® assay as described in Example 2). An example of an antigen of interest can be BNP, a BNP fragment, hBNP or a hBNP fragment. Such antibodies can be used as reagents to reduce heterophilic interference in an immunoassay.

j) Stringent Conditions

As used herein, the term “stringent conditions” refers to hybridization to filter-bound DNA in 6× sodium chloride/sodium citrate (SSC) at about 45° C. followed by one or more washes in 0.2×SSC/0.1% SDS at about 50-65° C. The term “under highly stringent conditions”, refers to hybridization to filter-bound nucleic acid in 6×SSC at about 45° C. followed by one or more washes in 0.1×SSC/0.2% SDS at about 68° C., or under other stringent hybridization conditions which are known to those skilled in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

k) Subject or Patient

As used herein, the terms “subject” and “patient” are used interchangeably. As used herein, the terms “subject” and “subjects” refer to an animal, in one aspect, a bird (for example, a duck or goose), in another aspect, a shark or whale, or in a further aspect, a mammal including, a non-primate (for example, a cow, pig, camel, llama, horse, goat, rabbit, sheep, hamsters, guinea pig, cat, dog, rat, and mouse) and a primate (for example, a monkey, such as a cynomolgous monkey, chimpanzee, and a human).

l) Test Sample

As used herein, the term “test sample” or “sample” generally refers to a biological material derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

The terminology used herein is for the purpose of describing particular embodiments only and is not otherwise intended to be limiting.

### B. Negative Mimic Antibodies

In one embodiment, the present disclosure relates to negative mimic antibodies or NEMIC antibodies. Specifically, the negative mimic antibodies of the present disclosure are antibodies that do not bind, more specifically, do not immunospecifically bind to BNP or any fragment thereof. More specifically, the antibodies of the present disclosure do not immunospecifically bind to human BNP or any hBNP fragment thereof. The antibodies of the present disclosure can be used as heterophilic blocking agents to reduce heterophilic interference in an immunoassay. Preferably, the immunoassay is an immunoassay for BNP or a BNP fragment, preferably, hBNP or a hBNP fragment thereof.

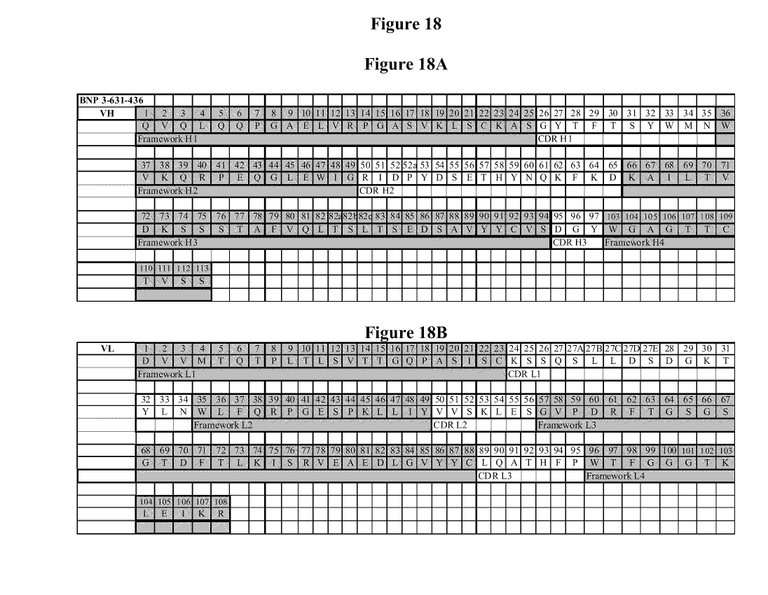

The negative mimic antibodies of the present disclosure comprise at least one mutation (such as deletions, additions and/or substitutions) in at least one of the heavy chain complementary determining (“CDR”) regions (for example, the heavy chain CDR 1, heavy chain CDR 2 and/or heavy chain CDR 3), at least one mutation (such as deletions, additions and/or substitutions) in the light chain CDR regions (for example, the light chain CDR 1, light chain CDR 2, and/or light chain CDR 3), at least one mutation (such as deletions, additions and/or substitutions) in the heavy chain framework regions (for example, the heavy chain framework 1, heavy chain framework 2, heavy chain framework 3 and/or heavy chain framework 4) and/or at least one mutation (such as deletions, additions and/or substitutions) in the light chain framework regions (for example, the light chain framework 1, light chain framework 2, light chain framework 3 and/or light chain framework 4) when compared to the amino acid sequence of the antibody produced by hybridoma cell line 3-631-436, specifically, hybridoma cell line 3-631-436, A.T.C.C. Accession No. PTA-6476 (also referred to herein as the “wildtype”; See, FIG. 18A and FIG. 18B and SEQ ID NOS:127 and 128). Moreover, the antibodies of the present disclosure may also contain one or more other mutations (such as deletions, additions and/or substitutions) in a part or portion of the antibody other than the CDR or framework region of the antibody. Methods for creating such derivatives are well known in the art and include the use of site-directed mutagenesis and PCR-mediated mutagenesis, which will be discussed in more detail infra.

More specifically, in another aspect, the antibodies of the present disclosure do not immunospecifically bind to BNP or a fragment thereof, preferably to hBNP or a hBNP fragment, and comprises a heavy chain CDR 1 having an amino acid sequence of the formula of:

(SEQ ID NO:1)

Gly-Tyr-Xaa₉-Phe-Thr-Xaa₁₀-Xaa₁₁-Xaa₁₂-Xaa₁₃-Xaa₁₄

wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine, provided that if (a) Xaa₉ is threonine, Xaa₁₀ is serine, Xaa₁₁ is tyrosine, Xaa₁₂ is tryptophan, Xaa₁₃ is methionine then Xaa₁₄ is glycine; (b) Xaa₁₀ is serine, Xaa₁₁ is tyrosine, Xaa₁₂ is tryptophan, Xaa₁₃ is methionine, Xaa₁₄ is alanine, then Xaa₉ is alanine; (c) Xaa₉ is threonine, Xaa₁₁ is tyrosine, Xaa₁₂ is tryptophan, Xaa₁₃ is methionine, Xaa₁₄ is asparagine, then Xaa₁₀ is alanine; (d) Xaa₉ is threonine, Xaa₁₀ is serine, Xaa₁₂ is tryptophan, Xaa₁₃ is methionine, Xaa₁₄ is asparagine, then Xaa₁₁ is aspartic acid or proline; (e) Xaa₉ is threonine, Xaa₁₀ is serine, Xaa₁₁ is tyrosine, Xaa₁₃ is methionine, Xaa₁₄ is asparagine then Xaa₁₂ is cysteine, glutamine, proline, valine or asparagine; or (f) Xaa₉ is threonine, Xaa₁₀ is serine, Xaa₁₁ is tyrosine, Xaa₁₂ is tryptophan, Xaa₁₄ is asparagine then Xaa₁₃ is proline, threonine, lysine or histidine.

In yet a further aspect, the antibody of the present disclosure does not immunospecifically bind to hBNP or hBNP fragment and comprises a heavy chain CDR 1 having the amino acid sequence of: Gly-Tyr-Thr-Phe-Thr-Ala-Tyr-Cys-Met-Asn (SEQ ID NO:102), Gly-Tyr-Thr-Phe-Thr-Ser-Tyr-Gln-Pro-Gly (SEQ ID NO:103), Gly-Tyr-Thr-Phe-Thr-Ser-Tyr-Pro-Thr-Gly (SEQ ID NO:104), Gly-Tyr-Thr-Phe-Thr-Ser-Asp-Val-Lys-Asn (SEQ ID NO:105), Gly-Tyr-Ala-Phe-Thr-Ser-Tyr-Trp-Met-Asn (SEQ ID NO:106) or Gly-Tyr-Thr-Phe-Thr-Ser-Pro-Asn-His-Asn (SEQ ID NO:107).

In another aspect, the present disclosure relates to an antibody that does not immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:102, SEQ ID NO:103, SEQ ID NO:104, SEQ ID NO:105, SEQ ID NO:106 or SEQ ID NO:107.

In another aspect, the antibody of the present disclosure do not immunospecifically bind to BNP or a fragment thereof, preferably to hBNP or a hBNP fragment, and comprises a heavy chain CDR 2 having an amino acid sequence of the formula of:

(SEQ ID NO:2)

Arg-Ile-Xaa₁-Xaa₂-Xaa₃-Xaa₄-Xaa₅-Glu-Thr-His-Tyr-
Asn-Gln-Lys-Phe-Lys-Asp

wherein Xaa₁ is aspartic acid or cysteine; Xaa₂ is proline or leucine; Xaa₃ is tyrosine, proline or methionine; Xaa₄ is aspartic acid or serine and Xaa₅ is serine or leucine, provided that if: (a) Xaa₁ is aspartic acid, Xaa₂ is proline, Xaa₃ is tyrosine, Xaa₄ is aspartic acid, then Xaa₅ is leucine; (b) Xaa₂ is proline, Xaa₃ is tyrosine, Xaa₄ is aspartic acid, Xaa₅ is serine, then Xaa₁ is cysteine; (c) Xaa₁ is aspartic acid, Xaa₃ is tyrosine, Xaa₄ is aspartic acid, Xaa₅ is serine, then Xaa₂ is leucine; or (d) Xaa₁ is aspartic acid, Xaa₂ is proline, Xaa₄ is aspartic acid, Xaa₅ is serine, then Xaa₃ is proline or methionine; or (e) Xaa₁ is aspartic acid, Xaa₂ is proline, Xaa₃ is tyrosine, Xaa₅ is serine, then Xaa₄ is serine.

In yet a further aspect, the antibody of the present disclosure does not immunospecifically bind to hBNP or hBNP fragment and comprises a heavy chain CDR 2 having the amino acid sequence of Arg-Ile-Asp-Pro-Pro-Ser-Lys-Glu-Thr-His-Tyr-Asn-Gln-Lys-Phe-Lys (SEQ ID NO:69), Arg-Ile-Cys-Leu-Met-Tyr-Asp-Ser-Glu-Thr-His-Tyr-Asn-Gln-Lys-Phe-Lys-Asp (SEQ ID NO:108) or Arg-Ile-Asp-Pro-Pro-Ser-Leu-Glu-Thr-His-Tyr-Asn-Gln-Lys-Phe-Lys-Asp (SEQ ID NO:129). In another aspect, the present disclosure relates to an antibody that does not immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:69, SEQ ID NO:108 or SEQ ID NO:129.

In yet another aspect, the antibody of the present disclosure does not immunospecifically bind to BNP or a fragment thereof, preferably, hBNP or a hBNP fragment, and comprises a light chain CDR 1 that has an amino acid sequence having a formula of:

(SEQ ID NO:4)

Xaa₁₅-Xaa₁₆-Xaa₁₇-Xaa₁₈-Xaa₁₉-Xaa₂₀-Leu-Asp-Ser-
Asp-Gly-Xaa₂₁-Xaa₂₂-Xaa₂₃-Leu-Asn

wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline, provided that if: (a) Xaa₁₅ is lysine; Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, then Xaa₂₃ is proline; (b) Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₅ is leucine; (c) Xaa₁₅ is lysine, Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₆ is valine or leucine; (d) Xaa₁₅ is lysine, Xaa₁₆ is serine; Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₇ is arginine, aspartic acid or threonine; (e) Xaa₁₅ is lysine, Xaa₁₆ is serine; Xaa₁₇ is serine, Xaa₁₉ is serine, Xaa₂₀ is leucine, Xaa₂₁ is lysine, Xaa₂₂ is threonine, Xaa₂₃ is tyrosine, then Xaa₁₈ is glutamic acid; (f) Xaa₁₅ is lysine, Xaa₁₆ is serine; Xaa₁₇ is serine, Xaa₁₈ is glutamine, Xaa₂₀ is leucine, Xaa₂₁ is lysine, Xaa₂₂ is threonine, Xaa₂₃ is tyrosine, then Xaa₁₉ is aspartic acid or leucine; (g) Xaa₁₅ is lysine, Xaa₁₆ is serine; Xaa₁₇ is serine, Xaa₁₈ is glutamine, Xaa₁₉ is serine, Xaa₂₁ is lysine, Xaa₂₂ is threonine, Xaa₂₃ is tyrosine, then Xaa₂₀ is glutamine; (h) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₂ is threonine, Xaa₂₃ is tyrosine, then Xaa₂₁ is glutamine; or (i) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₁ is lysine, Xaa₂₃ is tyrosine, then Xaa₂₂ is glycine.

In yet a further aspect, the antibody does not immunospecifically bind to hBNP or hBNP fragment and has a light chain CDR 1 having the amino acid sequence of:

(SEQ ID NO:109)

Lys-Val-Arg-Glu-Ser-Leu-Leu-Asp-Ser-Asp-Gly-Lys-
Thr-Tyr-Leu-Asn,

(SEQ ID NO:110)

Lys-Ser-Asp-Leu-Asp-Leu-Leu-Asp-Ser-Asp-Gly-Lys-
Thr-Tyr-Leu-Asn,

(SEQ ID NO:111)

Leu-Leu-Thr-Gln-Ser-Leu-Leu-Asp-Ser-Asp-Gly-Lys-
Thr-Tyr-Leu-Asn,

(SEQ ID NO:112)

Lys-Ser-Ser-Ser-Leu-Gln-Leu-Asp-Ser-Asp-Gly-Lys-
Thr-Tyr-Leu-Asn
or

(SEQ ID NO:113)

Lys-Ser-Ser-Gln-Ser-Leu-Leu-Asp-Ser-Asp-Gly-Gln-
Gly-Pro-Leu-Asn.

In another aspect, the present disclosure relates to an antibody that immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:109, SEQ ID NO:10, SEQ ID NO:111, SEQ ID NO:112 or SEQ ID NO:113.

In yet another aspect, the antibody of the present disclosure does not immunospecifically bind to BNP or a fragment thereof, preferably, hBNP or a hBNP fragment, and comprises a light chain CDR 2 that has an amino acid sequence having a formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine, provided that if (a) Xaa₆ is serine and Xaa₇ is lysine, then Xaa₈ is glutamine; (b) Xaa₇ is lysine and Xaa₈ is leucine, then Xaa₆ is glutamic acid; or (c) Xaa₆ is serine and Xaa₈ is leucine, then Xaa₇ is proline.

In yet a further aspect, the antibody does not immunospecifically bind to hBNP or hBNP fragment and has a light chain CDR 2 having the amino acid sequence of: Val-Val-Glu-Pro-Gln-Glu-Ser (SEQ ID NO:70). In another aspect, the present disclosure relates to an antibody that immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:70.

In yet a further aspect, the antibody of the present disclosure does not immunospecifically bind to BNP or a fragment thereof, preferably, hBNP or a hBNP fragment, and comprises a heavy chain framework 1 that has an amino acid sequence having a formula of:

(SEQ ID NO:114)

Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-
Ala-Ser

wherein Xaa₂₄ is alanine.

In yet a further aspect, the antibody does not immunospecifically bind to hBNP or hBNP fragment and has a heavy chain framework 1 having the amino acid sequence of: Gln-Val-Gln-Leu-Gln-Gln-Pro-Gly-Ala-Glu-Leu-Val-Arg-Pro-Gly-Ala-Ser-Ala-Lys-Leu-Ser-Cys-Lys-Ala-Ser (SEQ ID NO:115). In another aspect, the present disclosure relates to an antibody that immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:115.

In yet a further aspect, the antibody of the present disclosure does not immunospecifically bind to BNP or a fragment thereof, preferably, hBNP or a hBNP fragment, and comprises a heavy chain framework 2 that has an amino acid sequence having a formula of:

(SEQ ID NO:116)

Trp-Val-Lys-Gln-Arg-Pro-Glu-Gln-Gly-Leu-Glu-Trp-
Ile-Xaa₂₅

wherein Xaa₂₅ glutamic acid.

In yet a further aspect, the antibody does not immunospecifically bind to hBNP or hBNP fragment and has a heavy chain framework 2 having the amino acid sequence of: Trp-Val-Lys-Gln-Arg-Pro-Glu-Gln-Gly-Leu-Glu-Trp-Ile-Glu (SEQ ID NO:117).

In another aspect, the present disclosure relates to an antibody that immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:117.

In yet a further aspect, the antibody of the present disclosure does not immunospecifically bind to BNP or a fragment thereof, preferably, hBNP or a hBNP fragment, and comprises a light chain framework 2 that has an amino acid sequence having a formula of:

(SEQ ID NO:121)

Trp-Xaa₂₆-Phe-Gln-Xaa₂₇-Pro-Gly-Glu-Ser-Pro-Lys-
Leu-Leu-Ile-Tyr

wherein Xaa₂₆ is leucine or serine and Xaa₂₇ is arginine or glycine, provided that if (a) Xaa₂₆ is leucine, then Xaa₂₇ is glycine; or (b) Xaa₂₇ is arginine, then Xaa₂₆ is serine.

In yet a further aspect, the antibody does not immunospecifically bind to hBNP or hBNP fragment and has a light chain framework 2 having the amino acid sequence of: Trp-Ser-Phe-Gln-Arg-Pro-Gly-Glu-Ser-Pro-Lys-Leu-Leu-Ile-Tyr (SEQ ID NO:122) or Trp-Leu-Phe-Gln-Gly-Pro-Gly-Glu-Ser-Pro-Lys-Leu-Leu-Ile-Tyr (SEQ ID NO:123).

In another aspect, the present disclosure relates to an antibody that immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:122 or SEQ ID NO:123.

In yet a further aspect, the antibody of the present disclosure does not immunospecifically bind to BNP or a fragment thereof, preferably, hBNP or a hBNP fragment, and comprises a light chain framework 4 that has an amino acid sequence having a formula of:

(SEQ TD NO:125)

Trp-Thr-Phe-Gly-Gly-Gly-Thr-Lys-Leu-Glu-Ile-Lys-
Xaa₂₈

wherein Xaa₂₈ is tryptophan.

In yet a further aspect, the antibody does not immunospecifically bind to hBNP or hBNP fragment and has a light chain framework 4 having the amino acid sequence of: Trp-Thr-Phe-Gly-Gly-Gly-Thr-Lys-Leu-Glu-Ile-Lys-Trp (SEQ ID NO:126). In another aspect, the present disclosure relates to an antibody that immunospecifically binds to hBNP or hBNP fragment that comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of SEQ ID NO:126.

In yet a further aspect, the antibody of the present disclosure does not immunospecifically binds to BNP or a fragment thereof, preferably, hBNP or a hBNP fragment, and has a heavy chain CDR 1, heavy chain CDR 2, heavy chain CDR 3, a heavy chain framework 1, a heavy chain framework 2, a heavy chain framework 3, a heavy chain framework 4, a light chain CDR 1, a light chain CDR 2, a light variable CDR 3, a light chain framework 1, a light chain framework 2, a light chain framework 3 and a light chain framework 4 comprising the following amino acid sequences:

(a) the heavy chain CDR 1 has an amino acid sequence having a formula of:

(SEQ ID NO:1)

Gly-Tyr-Xaa₉-Phe-Thr-Xaa₁₀-Xaa₁₁-Xaa₁₂-Xaa₁₃-Xaa₁₄

wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine;

(b) the heavy chain CDR 2 has an amino acid sequence having a formula of:

(SEQ ID NO:2)

Arg-Ile-Xaa₁-Xaa₂-Xaa₃-Xaa₄-Xaa₅-Glu-Thr-His-Tyr-
Asn-Gln-Lys-Phe-Lys-Asp

wherein Xaa₁ is aspartic acid or cysteine; Xaa₂ is proline or leucine; Xaa₃ is tyrosine, proline or methionine; Xaa₄ is aspartic acid or serine and Xaa₅ is serine or leucine;

(c) the heavy chain CDR 3 has an amino acid sequence of:

| | |
|---|---|
| Asp-Gly-Tyr; | (SEQ ID NO:3) |

(d) the light chain CDR 1 has an amino acid sequence having a formula of: Xaa₁₅-Xaa₁₆-Xaa₁₇-Xaa₁₈-Xaa₁₉-Xaa₂₀-Leu-Asp-Ser-Asp-Gly-Xaa₂₁-Xaa₂₂-Xaa₂₃-Leu-Asn (SEQ ID NO:4)

wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline;

(e) the light chain CDR 2 has an amino acid sequence having the formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine;

(f) the light chain CDR 3 has an amino acid sequence having a formula of:

| | |
|---|---|
| Leu-Gln-Ala-Thr-His-Phe-Pro; | (SEQ ID NO:6) |

(g) the heavy chain framework 1 has an amino acid sequence having a formula of:

(SEQ ID NO:130)

Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-
Ala-Ser

wherein Xaa₂₄ is valine or alanine;

(h) the heavy chain framework 2 has an amino acid sequence having a formula of:

(SEQ ID NO:131)

Trp-Val-Lys-Gln-Arg-Pro-Glu-Gln-Gly-Leu-Glu-Trp-
Ile-Xaa₂₅

wherein Xaa₂₅ is glycine or glutamic acid;

(i) the heavy chain framework 3 has an amino acid sequence having a formula of:

(SEQ ID NO:118)

Lys-Ala-Ile-Leu-Thr-Val-Asp-Lys-Ser-Ser-Ser-Thr-
Ala-Phe-Val-Gln-Leu-Thr-Ser-Leu-Thr-Ser-Glu-Asp-
Ser-Ala-Val-Tyr-Tyr-Cys-Val-Ser;

(j) the heavy chain framework 4 has an amino acid sequence having a formula of:

(SEQ ID NO:119)

Trp-Gly-Ala-Gly-Thr-Thr-Cys-Thr-Val-Ser-Ser;

(k) the light chain framework 1 has an amino acid sequence having a formula of:

(SEQ ID NO:120)

Asp-Val-Val-Met-Thr-Gln-Thr-Pro-Leu-Thr-Leu-Ser-
Val-Thr-Thr-Gly-Gln-Pro-Ala-Ser-Ile-Ser-Cys;

(l) the light chain framework 2 has an amino acid sequence having a formula of:

(SEQ ID NO:121)

Trp-Xaa₂₆-Phe-Gln-Xaa₂₇-Pro-Gly-Glu-Ser-Pro-Lys-
Leu-Leu-Ile-Tyr

wherein Xaa₂₆ is leucine or serine;

wherein Xaa₂₇ is arginine or glycine;

(m) the light chain framework 3 has an amino acid sequence having a formula of:

(SEQ ID NO:124)

Gly-Val-Pro-Asp-Arg-Phe-Thr-Gly-Ser-Gly-Ser-Gly-
Thr-Asp-Phe-Thr-Leu-Lys-Ile-Ser-Arg-Val-Glu-Ala-
Glu-Asp-leu-Gly-Val-Tyr-Tyr-Cys;
and

(n) the light chain framework 4 has an amino acid sequence having a formula of:

(SEQ ID NO:132)

Trp-Thr-Phe-Gly-Gly-Gly-Thr-Lys-Leu-Glu-Ile-Lys-
Xaa₂₈

wherein Xaa₂₈ is arginine or tryptophan;

provided that each of Xaa₁-Xaa₂₈ are not simultaneously (e.g., meaning each at the same time) the following: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg. In other words, the heavy chain CDR 1, heavy chain CDR 2, a light chain CDR 1, a light chain CDR 2, a heavy chain framework 1, a heavy chain framework 2, a heavy chain framework 4, a light chain framework 2, and a light chain framework 4 shown above in SEQ ID NOS:1, 2, 4, 5, 114, 116, 119, 121 and 125 do not compass the wildtype sequences for hybridoma cell line 3-631-436 as show in FIG. 18A and FIG. 18B and in SEQ ID NOS:127 and 128.

Examples of antibodies having the above-described formulas comprise a heavy chain CDR 1, heavy chain CDR 2, heavy chain CDR 3, heavy chain framework 1, heavy chain framework 2, heavy chain framework 3, heavy chain framework 4, light chain CDR 1, light chain CDR 2, light chain CDR 3, light chain framework 1, light chain framework 2, light chain framework 3 and light chain framework 4 where Xaa₁-Xaa₂₈ in the above described formula are shown below in Table A.

TABLE A

| | CN1 | CN2 | CN4 | CN6 | CN7 | CN8 | CN9 |
|---|---|---|---|---|---|---|---|
| Xaa₁ | Asp | Cys | Asp | Asp | Asp | Asp | Asp |
| Xaa₂ | Pro | Leu | Pro | Pro | Pro | Pro | Pro |
| Xaa₃ | Tyr | Met | Pro | Tyr | Tyr | Pro | Tyr |
| Xaa₄ | Asp | Asp | Ser | Asp | Asp | Ser | Asp |
| Xaa₅ | Ser | Ser | Leu | Ser | Ser | Leu | Ser |
| Xaa₆ | Ser | Ser | Glu | Ser | Ser | Ser | Ser |
| Xaa₇ | Lys | Lys | Pro | Lys | Lys | Lys | Lys |
| Xaa₈ | Leu | Leu | Gln | Leu | Leu | Leu | Leu |
| Xaa₉ | Thr | Thr | Thr | Thr | Thr | Ala | Thr |
| Xaa₁₀ | Ala | Ser | Ser | Ser | Ser | Ser | Ser |
| Xaa₁₁ | Tyr | Tyr | Tyr | Tyr | Asp | Tyr | Pro |
| Xaa₁₂ | Cys | Gln | Trp | Pro | Val | Trp | Asn |
| Xaa₁₃ | Met | Pro | Met | Thr | Lys | Met | His |
| Xaa₁₄ | Asn | Gly | Asn | Gly | Asn | Asn | Asn |
| Xaa₁₅ | Lys | Lys | Lys | Leu | Lys | Lys | Lys |
| Xaa₁₆ | Val | Ser | Ser | Leu | Ser | Ser | Ser |
| Xaa₁₇ | Arg | Asp | Ser | Thr | Ser | Ser | Ser |
| Xaa₁₈ | Glu | Leu | Gln | Gln | Ser | Gln | Gln |
| Xaa₁₉ | Ser | Asp | Ser | Ser | Leu | Ser | Ser |
| Xaa₂₀ | Leu | Leu | Leu | Leu | Gln | Leu | Leu |
| Xaa₂₁ | Lys | Lys | Lys | Lys | Lys | Lys | Gln |
| Xaa₂₂ | Thr | Thr | Thr | Thr | Thr | Thr | Gly |
| Xaa₂₃ | Tyr | Tyr | Tyr | Tyr | Tyr | Tyr | Pro |
| Xaa₂₄ | Val | Val | Val | Val | Ala | Val | Val |
| Xaa₂₅ | Gly | Gly | Gly | Glu | Gly | Gly | Gly |
| Xaa₂₆ | Leu | Ser | Leu | Leu | Leu | Leu | Leu |
| Xaa₂₇ | Arg | Arg | Arg | Gly | Arg | Arg | Arg |
| Xaa₂₈ | Arg | Arg | Arg | Arg | Arg | Arg | Trp |

In another aspect, the present disclosure provides CN4 antibodies produced by Chinese hamster ovary (CHO) cell line BNP3-631-436CN4CHO. Antibodies produced by this cell line bind to do not immunospecifically bind to hBNP or a hBNP fragment. Specifically, these antibodies contain a heavy chain CDR2 having the amino acid sequence of Arg-Ile-Asp-Pro-Pro-Ser-Lys-Glu-Thr-His-Tyr-Asn-Gln-Lys-Phe-Lys (SEQ ID NO:69) and a light chain CDR 1 having the amino acid sequence of: Val-Val-Glu-Pro-Gln-Glu-Ser (SEQ ID NO:70).

### C. Nucleic Acid Molecules

The present disclosure provides for a nucleic acid molecule, generally isolated, encoding an antibody of the present disclosure that does not immunospecifically binds to BNP or a fragment thereof, specifically hBNP or a hBNP fragment.

As discussed previously herein, the antibodies of the present disclosure comprise at least one mutation (such as deletions, additions and/or substitutions) in at least one of the heavy chain complementary determining (CDR) regions (for example, the heavy chain CDR 1, heavy chain CDR 2, or heavy chain CDR 3), at least one mutation (such as deletions, additions and/or substitutions) in the light chain CDR regions (for example, the light chain CDR 1, light chain CDR 2, or light chain CDR 3), at least one mutation (such as deletions, additions and/or substitutions) in the heavy chain framework regions (for example, the heavy chain framework 1, heavy chain framework 2, heavy chain framework 3 and/or heavy chain framework 4) and/or at least one mutation (such as deletions, additions and/or substitutions) in the light chain framework regions (for example, the light chain framework 1, light chain framework 2, light chain framework 3 and/or light chain framework 4) when compared to the amino acid sequence the antibody produced by hybridoma cell line 3-631-436, A.T.C.C. Accession No. PTA-6476 (See, FIG. 18A and FIG. 18B and SEQ ID NOS:127 and 128). Standard techniques known to those of skill in the art can be used to introduce mutations (such as deletions, additions, and/or substitutions) in the nucleic acid molecule encoding an antibody of the present disclosure, including, for example, site-directed mutagenesis and PCR-mediated mutagenesis which results in amino acid substitutions. In one aspect, the derivatives include less than 10 amino acid substitutions, less than 5 amino acid substitutions, less than 4 amino acid substitutions, less than 3 amino acid substitutions, or less than 2 amino acid substitutions relative to the original antibody produced by hybridoma cell line 3-631-436. A “conservative amino acid substitution” is one in which the amino acid residue is replaced with the amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Mutations can be introduced randomly along all or part of the coding sequence, such as by saturation mutagenesis, and the resultant mutants can be screened for biological activity to identify mutants that do not exhibit any binding affinity to BNP or a fragment thereof, preferably, hBNP or a hBNP fragment. Following mutagenesis, the encoded antibody can be expressed and the activity of the antibody can be determined.

In another aspect, the present disclosure provides an isolated nucleic acid molecule encoding an antibody that does not immunospecifically bind to BNP or a fragment thereof, specifically, hBNP or a hBNP fragment, said antibody having a heavy chain CDR 1 having an amino acid sequence of the formula of:

(SEQ ID NO:1)

Gly-Tyr-Xaa₉-Phe-Thr-Xaa₁₀-Xaa₁₁-Xaa₁₂-Xaa₁₃-Xaa₁₄

wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine, provided that if (a) Xaa₉ is threonine, Xaa₁₀ is serine, Xaa₁₁ is tyrosine, Xaa₁₂ is tryptophan, Xaa₁₃ is methionine then Xaa₁₄ is glycine; (b) Xaa₁₀ is serine, Xaa₁₁ is tyrosine, Xaa₁₂ is tryptophan, Xaa₁₃ is methionine, Xaa₁₄ is alanine, then Xaa₉ is alanine; (c) Xaa₉ is threonine, Xaa₁₁ is tyrosine, Xaa₁₂ is tryptophan, Xaa₁₃ is methionine, Xaa₁₄ is asparagine, then Xaa₁₀ is alanine; (d) Xaa₉ is threonine, Xaa₁₀ is serine, Xaa₁₂ is tryptophan, Xaa₁₃ is methionine, Xaa₁₄ is asparagine, then Xaa₁₁ is aspartic acid or proline; (e) Xaa₉ is threonine, Xaa₁₀ is serine, Xaa₁₁ is tyrosine, Xaa₁₃ is methionine, Xaa₁₄ is asparagine then Xaa₁₂ is cysteine, glutamine, proline, valine or asparagine; or (f) Xaa₉ is threonine, Xaa₁₀ is serine, Xaa₁₁ is tyrosine, Xaa₁₂ is tryptophan, Xaa₁₄ is asparagine then Xaa₁₃ is proline, threonine, lysine or histidine.

The present disclosure also provides an isolated nucleic acid molecule that comprises a nucleotide sequence that hybridizes, under stringent conditions, to the nucleic acid molecule described herein that encodes an antibody having a heavy chain CDR 1 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a heavy chain CDR 1 having an amino acid sequence of SEQ ID NO:102, SEQ ID NO:103, SEQ ID NO:104, SEQ ID NO:105, SEQ ID NO:106 or SEQ ID NO:107. The present disclosure also provides an isolated nucleic acid molecule that comprises a nucleotide sequence that hybridizes, under stringent conditions, to the nucleic acid molecule described herein that encodes an antibody comprising a heavy chain CDR 1 having the amino acid sequence of SEQ ID NO:102, SEQ ID NO:103, SEQ ID NO:104, SEQ ID NO:105, SEQ ID NO:106 or SEQ ID NO:107.

In another aspect, the present disclosure provides an isolated nucleic acid molecule encoding an antibody that does not immunospecifically bind to BNP or a fragment thereof, specifically, hBNP or a hBNP fragment, said antibody having a heavy chain CDR 2 having an amino acid sequence of the formula of:

(SEQ ID NO:2)

Arg-Ile-Xaa₁-Xaa₂-Xaa₃-Xaa₄-Xaa₅-Glu-Thr-His-Tyr-
Asn-Gln-Lys-Phe-Lys-Asp

wherein Xaa₁ is aspartic acid or cysteine; Xaa₂ is proline or leucine; Xaa₃ is tyrosine, proline or methionine; Xaa₄ is aspartic acid or serine and Xaa₅ is serine or lysine, provided that if: (a) Xaa₁ is aspartic acid, Xaa₂ is proline, Xaa₃ is tyrosine, Xaa₄ is aspartic acid, then Xaa₅ is leucine; (b) Xaa₂ is proline, Xaa₃ is tyrosine, Xaa₄ is aspartic acid, Xaa₅ is serine, then Xaa₁ is cysteine; (c) Xaa₁ is aspartic acid, Xaa₃ is tyrosine, Xaa₄ is aspartic acid, Xaa₅ is serine, then Xaa₂ is leucine; or (d) Xaa₁ is aspartic acid, Xaa₂ is proline, Xaa₄ is aspartic acid, Xaa₅ is serine, then Xaa₃ is proline or methionine; or (e) Xaa₁ is aspartic acid, Xaa₂ is proline, Xaa₃ is tyrosine, Xaa₅ is serine, then Xaa₄ is serine.

The present disclosure also provides an isolated nucleic acid molecule that comprises a nucleotide sequence that hybridizes, under stringent conditions, to the nucleic acid molecule described herein that encodes an antibody having a heavy chain CDR 2 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a heavy chain CDR 2 having an amino acid sequence of SEQ ID NO:69, SEQ ID NO:108 or SEQ ID NO:129. The present disclosure also provides an isolated nucleic acid molecule that comprises a nucleotide sequence that hybridizes, under stringent conditions, to the nucleic acid molecule described herein that encodes an antibody comprising a heavy chain CDR 2 having the amino acid sequence of SEQ ID NO:69, SEQ ID NO:108 or SEQ ID NO:129.

In another aspect, the present disclosure provides an isolated nucleic acid molecule encoding an antibody that does not immunospecifically bind to BNP or a fragment thereof, specifically, hBNP or a hBNP fragment, said antibody having a light chain CDR 1 having an amino acid sequence of the formula of:

(SEQ ID NO:4)

Xaa₁₅-Xaa₁₆-Xaa₁₇-Xaa₁₈-Xaa₁₉-Xaa₂₀-Leu-Asp-Ser-
Asp-Gly-Xaa₂₁-Xaa₂₂-Xaa₂₃-Leu-Asn

wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline, provided that if (a) Xaa₁₅ is lysine; Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, then Xaa₂₃ is proline; (b) Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₅ is leucine; (c) Xaa₁₅ is lysine, Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₆ is valine or leucine; (d) Xaa₁₅ is lysine, Xaa₁₆ is serine; Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₇ is arginine, aspartic acid or threonine; (e) Xaa₁₅ is lysine, Xaa₁₆ is serine; Xaa₁₇ is serine, Xaa₁₉ is serine, Xaa₂₀ is leucine, Xaa₂₁ is lysine, Xaa₂₂ is threonine, Xaa₂₃ is tyrosine, then Xaa₁₈ is glutamic acid; (f) Xaa₁₅ is lysine, Xaa₁₆ is serine; Xaa₁₇ is serine, Xaa₁₈ is glutamine, Xaa₂₀ is leucine, Xaa₂₁ is lysine, Xaa₂₂ is threonine, Xaa₂₃ is tyrosine, then Xaa₁₉ is aspartic acid or leucine; (g) Xaa₁₅ is lysine, Xaa₁₆ is serine; Xaa₁₇ is serine, Xaa₁₈ is glutamine, Xaa₁₉ is serine, Xaa₂₁ is lysine, Xaa₂₂ is threonine, Xaa₂₃ is tyrosine, then Xaa₂₀ is glutamine; (h) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₂ is threonine, Xaa₂₃ is tyrosine, then Xaa₂₁ is glutamine; or (i) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₁ is lysine, Xaa₂₃ is tyrosine, then Xaa₂₂ is glycine.

The present disclosure also provides an isolated nucleic acid molecule that comprises a nucleotide sequence that hybridizes, under stringent conditions, to the nucleic acid molecule described herein that encodes an antibody having a light chain CDR 1 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a light chain CDR 1 having an amino acid sequence of SEQ ID NO:109, SEQ ID NO:110, SEQ ID NO:111, SEQ ID NO:112 or SEQ ID NO:113. The present disclosure also provides an isolated nucleic acid molecule that comprises a nucleotide sequence that hybridizes, under stringent conditions, to the nucleic acid molecule described herein that encodes an antibody comprising a light chain CDR 1 having the amino acid sequence of SEQ ID NO:109, SEQ ID NO:10, SEQ ID NO:111, SEQ ID NO:112 or SEQ ID NO:113.

In another aspect, the present disclosure provides an isolated nucleic acid molecule encoding an antibody that does not immunospecifically bind to BNP or a fragment thereof, specifically, hBNP or a hBNP fragment, said antibody having a light chain CDR 2 having an amino acid sequence of the formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine, provided that if (a) Xaa₆ is serine and Xaa₇ is lysine, then Xaa₈ is glutamine; (b) Xaa₇ is lysine and Xaa₈ is leucine, then Xaa₆ is glutamic acid; or (c) Xaa₆ is serine and Xaa₈ is leucine, then Xaa₇ is proline.

The present disclosure also provides an isolated nucleic acid molecule that comprises a nucleotide sequence that hybridizes, under stringent conditions, to the nucleic acid molecule described herein that encodes an antibody having a light chain CDR 2 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a light chain CDR 2 having an amino acid sequence of SEQ ID NO:70. The present disclosure also provides an isolated nucleic acid molecule that comprises a nucleotide sequence that hybridizes, under stringent conditions, to the nucleic acid molecule described herein that encodes an antibody comprising a light chain CDR 2 having the amino acid sequence of SEQ ID NO:70.

In another aspect, the present disclosure provides an isolated nucleic acid molecule encoding an antibody that does not immunospecifically bind to BNP or a fragment thereof, specifically, hBNP or a hBNP fragment, said antibody having a heavy chain framework 1 having an amino acid sequence of the formula of:

(SEQ ID NO:114)

Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-Ala-
Ser

wherein Xaa₂₄ is alanine.

The present disclosure also provides an isolated nucleic acid molecule that comprises a nucleotide sequence that hybridizes, under stringent conditions, to the nucleic acid molecule described herein that encodes an antibody having a heavy chain framework 1 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a heavy chain framework 1 having an amino acid sequence of SEQ ID NO:115. The present disclosure also provides an isolated nucleic acid molecule that comprises a nucleotide sequence that hybridizes, under stringent conditions, to the nucleic acid molecule described herein that encodes an antibody comprising a heavy chain framework having the amino acid sequence of SEQ ID NO:115.

In another aspect, the present disclosure provides an isolated nucleic acid molecule encoding an antibody that does not immunospecifically bind to BNP or a fragment thereof, specifically, hBNP or a hBNP fragment, said antibody having a heavy chain framework 2 having an amino acid sequence of the formula of:

(SEQ ID NO:116)

Trp-Val-Lys-Gln-Arg-Pro-Glu-Gln-Gly-Leu-Glu-Trp-
Ile-Xaa₂₅

wherein Xaa₂₅ glutamic acid.

The present disclosure also provides an isolated nucleic acid molecule that comprises a nucleotide sequence that hybridizes, under stringent conditions, to the nucleic acid molecule described herein that encodes an antibody having a heavy chain framework 2 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a heavy chain framework 2 having an amino acid sequence of SEQ ID NO:117. The present disclosure also provides an isolated nucleic acid molecule that comprises a nucleotide sequence that hybridizes, under stringent conditions, to the nucleic acid molecule described herein that encodes an antibody comprising a heavy chain framework 2 having the amino acid sequence of SEQ ID NO:117.

In another aspect, the present disclosure provides an isolated nucleic acid molecule encoding an antibody that does not immunospecifically bind to BNP or a fragment thereof, specifically, hBNP or a hBNP fragment, said antibody having a light chain framework 2 having an amino acid sequence of the formula of:

(SEQ ID NO:121)

Trp-Xaa₂₆-Phe-Gln-Xaa₂₇-Pro-Gly-Glu-Ser-Pro-Lys-
Leu-Leu-Ile-Tyr

wherein Xaa₂₆ is leucine or serine and Xaa₂₇ is arginine or glycine, provided that if (a) Xaa₂₆ is leucine, then Xaa₂₇ is glycine; or (b) Xaa₂₇ is arginine, then Xaa₂₆ is serine.

The present disclosure also provides an isolated nucleic acid molecule that comprises a nucleotide sequence that hybridizes, under stringent conditions, to the nucleic acid molecule described herein that encodes an antibody having a light chain framework 2 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a light chain framework 2 having an amino acid sequence of SEQ ID NO:122 or SEQ ID NO:123. The present disclosure also provides an isolated nucleic acid molecule that comprises a nucleotide sequence that hybridizes, under stringent conditions, to the nucleic acid molecule described herein that encodes an antibody comprising a light chain framework 2 having the amino acid sequence of SEQ ID NO:122 or SEQ ID NO:123.

In another aspect, the present disclosure provides an isolated nucleic acid molecule encoding an antibody that does not immunospecifically bind to BNP or a fragment thereof, specifically, hBNP or a hBNP fragment, said antibody having a light chain framework 4 having an amino acid sequence of the formula of:

(SEQ ID NO:125)

Trp-Thr-Phe-Gly-Gly-Gly-Thr-Lys-Leu-Glu-Ile-Lys-
Xaa₂₈

wherein Xaa₂₈ is tryptophan.

The present disclosure also provides an isolated nucleic acid molecule that comprises a nucleotide sequence that hybridizes, under stringent conditions, to the nucleic acid molecule described herein that encodes an antibody having a light chain framework 4 having an amino acid sequence of the above-described formula.

In another aspect, the disclosure provides an isolated nucleic acid molecule encoding an antibody that does not immunospecifically bind to BNP or a fragment thereof, preferably, hBNP or a hBNP fragment, said antibody comprising (alternatively, consisting of) a light chain framework 4 having an amino acid sequence of SEQ ID NO:126. The present disclosure also provides an isolated nucleic acid molecule that comprises a nucleotide sequence that hybridizes, under stringent conditions, to the nucleic acid molecule described herein that encodes an antibody comprising a light chain framework 4 having the amino acid sequence of SEQ ID NO:126.

In another aspect, the present disclosure provides an isolated nucleic acid molecule encoding an antibody that does not immunospecifically bind to BNP or a fragment thereof, specifically, hBNP or a hBNP fragment, said antibody having a heavy chain CDR 1, heavy chain CDR 2, heavy chain CDR 3, a heavy chain framework 1, a heavy chain framework 2, a heavy chain framework 3, a heavy chain framework 4, a light chain CDR 1, a light chain CDR 2, a light variable CDR 3, a light chain framework 1, a light chain framework 2, a light chain framework 3 and a light chain framework 4 comprising the following amino acid sequences:

(a) the heavy chain CDR 1 has an amino acid sequence having a formula of:

(SEQ ID NO:1)

Gly-Tyr-Xaa₉-Phe-Thr-Xaa₁₀-Xaa₁₁-Xaa₁₂-Xaa₁₃-Xaa₁₄

wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine;

(b) the heavy chain CDR 2 has an amino acid sequence having a formula of:

(SEQ ID NO:2)

Arg-Ile-Xaa₁-Xaa₂-Xaa₃-Xaa₄-Xaa₅-Glu-Thr-His-Tyr-
Asn-Gln-Lys-Phe-Lys-Asp

wherein Xaa₁ is aspartic acid or cysteine; Xaa₂ is proline or leucine; Xaa₃ is tyrosine, proline or methionine; Xaa₄ is aspartic acid or serine and Xaa₅ is serine or leucine;

(c) the heavy chain CDR 3 has an amino acid sequence of:

| | |
|---|---|
| Asp-Gly-Tyr; | (SEQ ID NO:3) |

(d) the light chain CDR 1 has an amino acid sequence having a formula of:

(SEQ ID NO:4)

Xaa₁₅-Xaa₁₆-Xaa₁₇-Xaa₁₈-Xaa₁₉-Xaa₂₀-Leu-Asp-Ser-
Asp-Gly-Xaa₂₁-Xaa₂₂-Xaa₂₃-Leu-Asn

wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline;

(e) the light chain CDR 2 has an amino acid sequence having the formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine;

(f) the light chain CDR 3 has an amino acid sequence having a formula of:

| | |
|---|---|
| Leu-Gln-Ala-Thr-His-Phe-Pro; | (SEQ ID NO:6) |

(g) the heavy chain framework 1 has an amino acid sequence having a formula of:

(SEQ ID NO:130)

Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-
Ala-Ser

wherein Xaa₂₄ is valine or alanine;

(h) the heavy chain framework 2 has an amino acid sequence having a formula of:

(SEQ ID NO:131)

Trp-Val-Lys-Gln-Arg-Pro-Glu-Gln-Gly-Leu-Glu-Trp-
Ile-Xaa₂₅

wherein Xaa₂₅ is glycine or glutamic acid;

(i) the heavy chain framework 3 has an amino acid sequence having a formula of:

(SEQ ID NO:118)

Lys-Ala-Ile-Leu-Thr-Val-Asp-Lys-Ser-Ser-Ser-Thr-
Ala-Phe-Val-Gln-Leu-Thr-Ser-Leu-Thr-Ser-Glu-Asp-
Ser-Ala-Val-Tyr-Tyr-Cys-Val-Ser;

(j) the heavy chain framework 4 has an amino acid sequence having a formula of:

(SEQ ID NO:119)

Trp-Gly-Ala-Gly-Thr-Thr-Cys-Thr-Val-Ser-Ser;

(k) the light chain framework 1 has an amino acid sequence having a formula of:

(SEQ ID NO:120)

Asp-Val-Val-Met-Thr-Gln-Thr-Pro-Leu-Thr-Leu-Ser-
Val-Thr-Thr-Gly-Gln-Pro-Ala-Ser-Ile-Ser-Cys;

(l) the light chain framework 2 has an amino acid sequence having a formula of:

(SEQ ID NO:121)

Trp-Xaa₂₆-Phe-Gln-Xaa₂₇-Pro-Gly-Glu-Ser-Pro-Lys-
Leu-Leu-Ile-Tyr

wherein Xaa₂₆ is leucine or serine;

wherein Xaa₂₇ is arginine or glycine;

(m) the light chain framework 3 has an amino acid sequence having a formula of:

(SEQ ID NO:124)

Gly-Val-Pro-Asp-Arg-Phe-Thr-Gly-Ser-Gly-Ser-Gly-
Thr-Asp-Phe-Thr-leu-Lys-Ile-Ser-Arg-Val-Glu-Ala-
Glu-Asp-leu-Gly-Val-Tyr-Tyr-Cys;
and

(n) the light chain framework 4 has an amino acid sequence having a formula of:

(SEQ ID NO:132)

Trp-Thr-Phe-Gly-Gly-Gly-Thr-Lys-Leu-Glu-Ile-Lys-
Xaa₂₈

wherein Xaa₂₈ is arginine or tryptophan;

provided that each of Xaa₁-Xaa₂₈ are not simultaneously (e.g., meaning each at the same time) the following: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg. In other words, the heavy chain CDR 1, heavy chain CDR 2, a light chain CDR 1, a light chain CDR 2, a heavy chain framework 1, a heavy chain framework 2, a heavy chain framework 4, a light chain framework 2, and a light chain framework 4 shown above in SEQ ID NOS:1, 2, 4, 5, 114, 116, 119, 121 and 125 do not compass the wildtype sequences for hybridoma cell line 3-631-436 as show in FIG. 18A and FIG. 18B and in SEQ ID NOS:127 and 128.

The present disclosure also provides an isolated nucleic acid molecule that comprises a nucleotide sequence that hybridizes, under stringent conditions, to the nucleic acid molecule described herein that encodes an antibody having a heavy chain CDR 1 region, a heavy chain CDR 2 region, a heavy chain CDR 3 region, a heavy chain framework 1, a heavy chain framework 2, a heavy chain framework 3, a heavy chain framework 4, a light chain CDR 1 region, a light chain CDR 2 region, a light chain CDR 3 region, a light chain framework 1, a light chain framework 2, a light chain framework 3 and a light chain framework 4 having the amino acid sequences pursuant to the above-described formula.

In yet another aspect, the present disclosure provides an isolated nucleic acid molecule encoding an antibody that does not immunospecifically bind to hBNP or hBNP fragment, wherein said antibody is a CN4 antibody produced by CHO cell line BNP3-631-436CN4CHO. This nucleic acid can comprise at least one polynucleotide sequence selected from the group consisting of SEQ ID NO:82, SEQ ID NO:83, SEQ ID NO:84, SEQ ID NO:85 and combinations thereof. The present disclosure also provides an isolated nucleic acid molecule that comprises a nucleotide sequence that hybridizes, under stringent conditions, to the nucleic acid molecule that encodes an antibody that immunospecifically binds to hBNP or hBNP fragment, wherein said antibody is produced by CHO cell line BNP3-631-436CN4CHO.

In yet another aspect, the present disclosures provides an isolated polynucleotide comprising or consisting of a sequence selected from the group consisting of: SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101 and full complements thereof.

In still yet another aspect, the present disclosure provides an isolated nucleic acid molecule encoding an antibody that does not immunospecifically bind to hBNP or hBNP fragment, wherein said antibody is a CN1 antibody and further wherein said antibody has (a) a CDR heavy chain comprising or consisting of the polynucleotide sequence of SEQ ID NO:74, SEQ ID NO:75 or full complements thereof; and (b) a CDR light chain comprising or consisting of the polynucleotide sequence of SEQ ID NO:76, SEQ ID NO:77 or full complements thereof.

In still yet another aspect, the present disclosure provides an isolated nucleic acid molecule encoding an antibody that does not immunospecifically bind to hBNP or hBNP fragment, wherein said antibody is a CN2 antibody and further wherein said antibody has (a) a CDR heavy chain comprising or consisting of the polynucleotide sequence of SEQ ID NO:78, SEQ ID NO:79 or full complements thereof; and (b) a CDR light chain comprising or consisting of the polynucleotide sequence of SEQ ID NO:80, SEQ ID NO:81 or full complements thereof.

In still yet another aspect, the present disclosure provides an isolated nucleic acid molecule encoding an antibody that does not immunospecifically bind to hBNP or hBNP fragment, wherein said antibody is a CN4 antibody and further wherein said antibody has (a) a CDR heavy chain comprising or consisting of the polynucleotide sequence of SEQ ID NO:82, SEQ ID NO:83 or full complements thereof; and (b) a CDR light chain comprising or consisting of the polynucleotide sequence of SEQ ID NO:84, SEQ ID NO:85 or full complements thereof.

In still yet another aspect, the present disclosure provides an isolated nucleic acid molecule encoding an antibody that does not immunospecifically bind to hBNP or hBNP fragment, wherein said antibody is a CN6 antibody and further wherein said antibody has (a) a CDR heavy chain comprising or consisting of the polynucleotide sequence of SEQ ID NO:86, SEQ ID NO:87 or full complements thereof; and (b) a CDR light chain comprising or consisting of the polynucleotide sequence of SEQ ID NO:88, SEQ ID NO:89 or full complements thereof.

In still yet another aspect, the present disclosure provides an isolated nucleic acid molecule encoding an antibody that does not immunospecifically bind to hBNP or hBNP fragment, wherein said antibody is a CN7 antibody and further wherein said antibody has (a) a CDR heavy chain comprising or consisting of the polynucleotide sequence of SEQ ID NO:90, SEQ ID NO:91 or full complements thereof; and (b) a CDR light chain comprising or consisting of the polynucleotide sequence of SEQ ID NO:92, SEQ ID NO:93 or full complements thereof.

In still yet another aspect, the present disclosure provides an isolated nucleic acid molecule encoding an antibody that does not immunospecifically bind to hBNP or hBNP fragment, wherein said antibody is a CN8 antibody and further wherein said antibody has (a) a CDR heavy chain comprising or consisting of the polynucleotide sequence of SEQ ID NO:94, SEQ ID NO:95 or full complements thereof; and (b) a CDR light chain comprising or consisting of the polynucleotide sequence of SEQ ID NO:96, SEQ ID NO:97 or full complements thereof.

In still yet another aspect, the present disclosure provides an isolated nucleic acid molecule encoding an antibody that does not immunospecifically bind to hBNP or hBNP fragment, wherein said antibody is a CN9 antibody and further wherein said antibody has (a) a CDR heavy chain comprising or consisting of the polynucleotide sequence of SEQ ID NO:98, SEQ ID NO:99 or full complements thereof; and (b) a CDR light chain comprising or consisting of the polynucleotide sequence of SEQ ID NO:100, SEQ ID NO:101 or full complements thereof.

### D. Methods for Preparing Negative Mimic Antibodies

The antibodies of the present disclosure can be prepared using routine techniques known to those skilled in the art.

In one aspect, the antibodies of the present disclosure can be prepared by recombinant expression of immunoglobulin light and heavy chain genes in a host cell. To express an antibody recombinantly, a host cell is transfected with one or more recombinant expression vectors carrying nucleic acid molecules encoding the immunoglobulin light and heavy chains of the antibody such that the light and heavy chains are expressed in the host cell and, preferably, secreted into the medium in which the host cells are cultures, from which medium the antibodies can be recovered. Standard recombinant nucleic acid (DNA) methodologies are used to obtain antibody heavy and light chain genes, incorporate these genes into recombinant expressions vectors and introduce the vectors into host cells, such as those described in Sambrook, Fritsch and Maniatis (eds), Molecular Cloning: A Laboratory Manual, Second Edition, Cold Spring Harbor, New Your, (1989), Ausubel, F. M. et al. (eds.) Current Protocols in Molecular Biology, Greene Publishing Associates (1989) and in U.S. Pat. No. 4,816,397 by Boss et al.

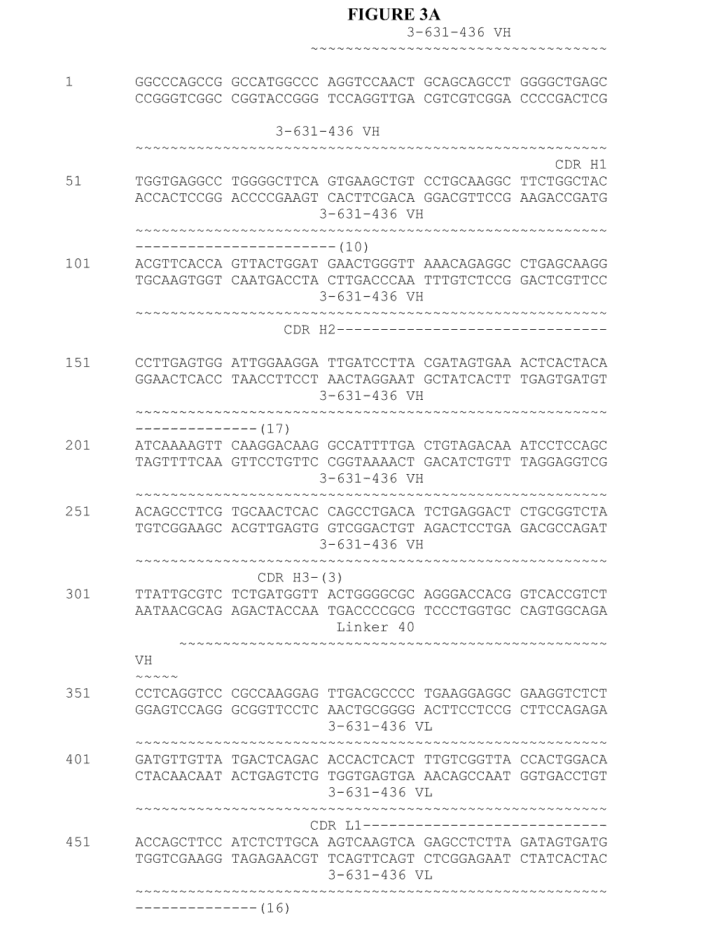

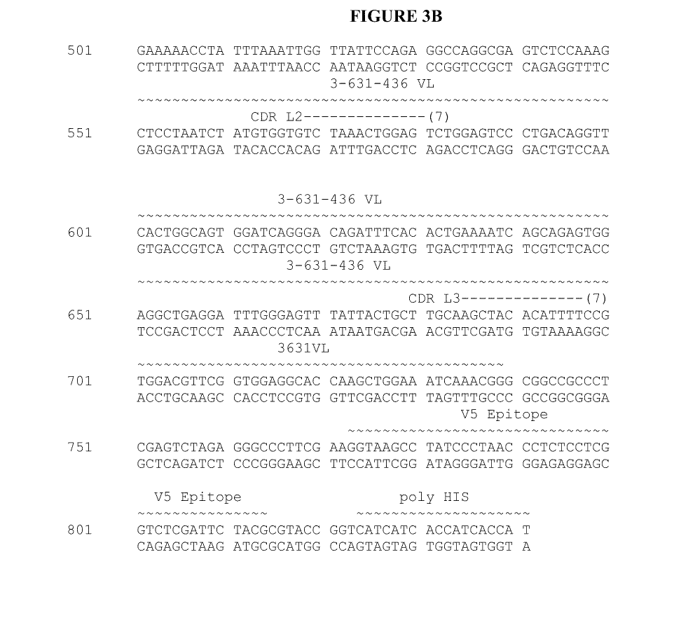

To express the antibodies of the disclosure, nucleic acid molecules encoding the light and heavy chain regions are first obtained. These nucleic acid molecules may be obtained from a hybridoma cell line monoclonal antibody 3-631-436 and modified by means well known in the art (such as site-directed mutagenesis) to generate antibodies of the present disclosure, including, for example, the antibodies produced by CHO cell line BNP3-631-436CN4CHO. A hybridoma cell line expressing monoclonal antibody 3-631-436 which was deposited with the American Type Culture Collection (A.T.C.C.) on Dec. 21, 2004 and assigned A.T.C.C. Accession No. PTA-6476 and is described in U.S. Patent Publication 2006/0183154 published on Aug. 17, 2006. The nucleic acid sequence of monoclonal antibody 3-631-436 is shown in FIGS. 3A-3B and SEQ ID NO: 73.

For example, once the 3-631-436 variable heavy (VH) and variable (VL) nucleic acid fragments are obtained, these sequences or specific regions within these sequences, such as the complementary determining (CDR) or framework regions, can be mutated to encode the CN1, CN2, CN4, CN6, CN7, CN8 or CN9 (referred to collectively herein as “CN1-CN9”)-related amino acid sequences disclosed herein. The amino acid sequences encoded by the 3-631-436 VH and VL DNA sequences are compared to the appropriate CN1-CN9 or CN1-CN9-related VH and VL amino acid sequences to identify amino acid residues in the CN1-CN9 or CN1-CN9-related sequence that differ. The appropriate nucleotides of antibody 3-631-436 are mutated such that the mutated sequence encodes the CN1-CN9 or CN1-CN9-related amino acid sequence, using the genetic code to determine which nucleotide changes should be made. Mutagenesis of antibody 3-631-436 sequences can be carried out by standard methods, such as PCR-mediated mutageneisis (in which the mutated nucleotides are incorporated into the PCR primers such that the PCR product contains the mutations) or site-directed mutagenesis.

Alternatively, in another aspect, nucleic acid molecules encoding the VH and VL chains can be synthesized on a chemical synthesizer, using routine techniques known to those in the art. For example, the VH and VL chains from the nucleic acid molecules described in Section C can be chemically synthesized using routine techniques known in the art. Starting at the 3′ terminal base which is attached to a support, nucleotides are coupled in a step-wise fashion. Following the addition of the most 5′ nucleotide, the nucleotide is cleaved from the solid support and purified by desalting followed by polyacrylamide gel electrophoresis (PAGE) (Midland Certified Reagents, Midland, Tex.).

Once nucleic acid fragments encoding CN1-CN9 or CN1-CN9-related VH and VL segments are obtained (by amplification and mutagenesis of VH and VL genes, as described above), these nucleic acid fragments can be further manipulated by standard recombinant DNA techniques, for example to convert the variable region genes to an antibody (such as, but not limited to, a full-length antibody chain genes, to Fab fragment genes or to a scFv gene). In these manipulations, a VL- or VH-encoding nucleic acid fragment is operatively linked to another nucleic acid fragment encoding another protein, such as antibody constant region or a flexible linker. The term “operatively linked”, as used in this context, is intended to mean that the two nucleic acid fragments are joined such that the amino acid sequences encoded by the two nucleic acid fragments remain in-frame.

In an alternative method, a scFv gene may be constructed with wildtype CDR regions (such as those of antibody 3-631-436) and then mutated using techniques known in the art.

The isolated nucleic acid molecule encoding the VH region can be converted to a full-length heavy chain gene by operatively linking the VH-encoding nucleic acid molecule to another nucleic acid molecule encoding heavy chain constant regions (CH1, CH2 and CH3). The sequences of human heavy chain constant region genes are known in the art (See for example, Kabat, E. A., et al., Sequences of Proteins of Immunological Interest, Fifth Edition, U.S. Department of Health and Human Services, NIH Publication No. 91-3242 (1991)). In another aspect, the present disclosure further encompasses all known human heavy chain constant regions, including but not limited to, all known allotypes of the human heavy chain constant region. Nucleic acid fragments encompassing these regions can be obtained by standard PCR amplification. The heavy chain constant region can be an IgG1, IgG2, IgG3, IgG4, IgA, IgE, IgM or IgD constant region.

The isolated nucleic acid molecule encoding the VL region can be converted to a full-length light chain gene (as well as a Fab light chain gene) by operatively linking the VL-encoding nucleic acid molecule to another nucleic acid molecule encoding the light chain constant region, CL. The sequences of human light chain constant region genes are known in the art (See, e.g., Kabat, E. A., et al., Sequences of Proteins of Immunological Interest, Fifth Edition, U.S. Department of Health and Human Services, NIH Publication No. 91-3242 (1991)). The present disclosure encompasses all known human light chain constant regions, including but not limited to, all known allotypes of the human light chain constant region. Nucleic acid fragments encompassing these regions can be obtained by standard PCR amplification. The light chain constant region can be a kappa or lambda constant region, but most preferably is a kappa constant region.

It is to be understood that the specific designations of framework (FR) and CDR regions within a particular heavy or light chain region may vary depending on the convention or numbering system used to identify such regions (e.g. Chothia, Kabat, Oxford Molecular's AbM modeling software, all of which are known to those of ordinary skill in the art). For the purposes of the present disclosure, the Kabat and numbering system is used. CDRs are defined using the Kabat and Molecular's AbM systems.

To create a scFv gene, the VH- and VL-encoding nucleic acid fragments are operatively linked to another fragment encoding a flexible linker, such as, a linker that is encoded by the amino acid sequence GPAKELTPLKEAKVS (SEQ ID NO:9). Examples of other linker sequences that can be used in the present disclosure can be found in Bird et al., Science 242:423-426 (1988), Huston et al., Proc. Natl. Acad. Sci. USA 85:5879-5883 (1988) and McCafferty et al., Nature, 348:552-554 (1990).

To express the antibodies, or antibody portions of the disclosure, nucleic acid molecules encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (for example, ligation of complementary restriction sites on the antibody gene fragment and vector, or blunt end ligation if no restriction sites are present). Prior to the insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operatively linked to the CH “segment” within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The single peptide can be an immunoglobin signal peptide or a heterologous signal peptide (i.e., a signal peptide from a non-immunoglobulin protein).

In addition to the antibody chain genes, the recombinant expression vectors can carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of the expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (such as the adenovirus major late promoter (AdMLP)) and polyoma. For further description of viral regulatory elements, and sequences thereof, see for example, U.S. Pat. No. 5,168,062, U.S. Pat. No. 4,510,245 and U.S. Pat. No. 4,968,615.

In addition to the antibody chain genes and regulatory sequences, recombinant expression vectors may carry additional sequences, such as sequences that regulate replication of the vector in host cells (e.g., origins of replication) and selectable marker genes. The selectable marker gene facilitates selection of host cells into which the vector has been introduced (See, for example, U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene for use in dhfr- host cells with methotrexate selection/amplification and the neomycin (“neo”) gene for G418 selection.

For expression of the light and heavy chains, the expression vector(s) encoding the heavy and light chains are transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (See, Boss, M. A. and Wood, C. R., Immunology Today 6:12-13 (1985)).

Preferred mammalian host cells for expressing the recombinant antibodies of the disclosure include the Chinese Hamster Ovary (CHO) cells (including dhfr-CHO cells, described in Urlaub and Chasin, Proc. Natl. Acad. Sci. USA 77:4216-4220 (1980), used with a DHFR selectable marker, for example, as described in R. J. Kaufman and P. A. Sharp, Mol. Biol. 159:601-621 (1982)), NSO myeloma cells, COS cells, HEK-293 cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments, F(ab′)₂ fragments or scFv molecules. It will be understood that variations on the above procedure are within the scope of the present disclosure. For example, it may be desirable to transfect a host cell with nucleic acid molecule encoding either the light chain or the heavy chain (but not both) of an antibody of the present disclosure. Recombinant DNA technology may also be used to remove some or all of the nucleic acid molecules encoding either or both of the light and heavy chains that one skilled in the art believes are not necessary for the antibodies of the present disclosure. The molecules expressed from such truncated nucleic acid molecules also are encompassed by the antibodies of the disclosure.

In a preferred system for recombinant expression of an antibody of the present disclosure, a recombinant expression vector encoding both the antibody heavy chain and the antibody light chain is introduced into dhfr deficient (dhfr-) CHO cells by lipofectamine mediated transfection (Invitrogen). Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector. Cells were cultured in medium without hypoxanthine and thymidine to obtain those CHO cells that have acquired the DHFR gene from the transfecting vector. Methyltrexate was supplemented into HT—media to improve antibody expression levels by causing gene duplication of dhfr gene and in turn antibody genes. Screening methods were used to identify those clones that expressed the highest quantity of antibody. Those individual clones were expanded and were routinely re-screened. Cell lines were selected from CN4 transfections. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of forgoing, another aspect of the disclosure pertains to nucleic acid, vector and host cell compositions that can be used for recombinant expression of the antibodies and antibody portions of the disclosure. In one aspect, the amino acid sequence encoding the heavy chain CDR 1 region of CN1 and variants thereof is shown in SEQ ID NO:102. In another aspect, the amino acid sequence encoding the light chain CDR 1 region of CN1 and variants thereof is shown in SEQ ID NO:109.

In another aspect, the amino acid sequence encoding the heavy chain CDR 1 region of CN2 and variants thereof is shown in SEQ ID NO:103. In another aspect, the amino acid sequence encoding the heavy chain CDR 2 region of CN2 and variants thereof is shown in SEQ ID NO:108. In another aspect, the amino acid sequence encoding the light chain CDR 1 region of CN2 and variants thereof is shown in SEQ ID NO:110. In yet another aspect, the amino acid sequence encoding the light chain framework 2 region of CN2 and variants thereof is shown in SEQ ID NO:122.

In still yet another aspect, the amino acid sequence encoding the heavy chain CDR 2 region of CN4 and variants thereof is shown in SEQ ID NO:69. In another aspect, the amino acid sequence encoding the light chain CDR 2 region of CN4 and variants thereof is shown in SEQ ID NO:70.

In still yet another aspect, the amino acid sequence encoding the heavy chain CDR 1 region of CN6 and variants thereof is shown in SEQ ID NO:104. In another aspect, the amino acid sequence encoding the heavy chain framework 2 region of CN6 and variants thereof is shown in SEQ ID NO:117. In another aspect, the amino acid sequence encoding the light chain CDR 1 region of CN6 and variants thereof is shown in SEQ ID NO:111. In yet another aspect, the amino acid sequence encoding the light chain framework 2 region of CN6 and variants thereof is shown in SEQ ID NO:123.

In still yet another aspect, the amino acid sequence encoding the heavy chain framework 1 region of CN7 and variants thereof is shown in SEQ ID NO:115. In another aspect, the amino acid sequence encoding the heavy chain CDR 1 region of CN7 and variants thereof is shown in SEQ ID NO:105. In another aspect, the amino acid sequence encoding the light chain CDR 1 region of CN7 and variants thereof is shown in SEQ ID NO:112.

In still yet another aspect, the amino acid sequence encoding the heavy chain CDR 1 region of CN8 and variants thereof is shown in SEQ ID NO:106. In another aspect, the amino acid sequence encoding the heavy chain CDR 2 region of CN8 and variants thereof is shown in SEQ ID NO:129.

In still yet another aspect, the amino acid sequence encoding the heavy chain CDR 1 region of CN9 and variants thereof is shown in SEQ ID NO:107. In another aspect, the amino acid sequence encoding the light chain CDR 1 region of CN9 and variants thereof is shown in SEQ ID NO:113. In yet another aspect, the amino acid sequence encoding the light chain framework 4 region is shown in SEQ ID NO:126.

### E. Selection of Recombinant Antibodies

The antibodies of the present disclosure, including the CN1-CN9 and CN1-CN9 related antibodies disclosed herein, can be isolated by screening of a combinatorial antibody library. Preferably, the combinatorial antibody library is a recombinant combinatorial library, preferably a scFv yeast display library, prepared using chimeric, humanized or human VL, VH and/or framework region cDNAs. Methodologies for preparing and screening such libraries are known in the art. In addition to commercially available vectors for generating yeast display libraries (such as, the pYD1 vector, Invitrogen, Carlsbad, Calif.) examples of methods and reagents particularly amenable for use in generating and screening antibody display libraries can be found in, for example, Boder E. T. and Wittrup K. D., Yeast surface display for directed evolution of protein expression, affinity, and stability, Methods Enzymol., 328:430-44 (2000) and Boder E. T. and Wittrup K. D., Yeast surface display for screening combinatorial polypeptide libraries, Nat Biotechnol. 15(6):553-7 (June 1997).

In a preferred embodiment, to isolate antibodies that do not immunospecifically bind to BNP or a fragment thereof, specifically hBNP or a fragment of hBNP, such as any of the antibodies described in Section B herein, an antibody that is known to immunospecifically bind to BNP, specifically, hBNP or hBNP fragment (such as, for example, antibody 3-631-436) is first used to generate mouse heavy and light chain sequences expressed as scFvs on the surface of yeast (preferably, Saccaromyces cerevisiae). These antibody (such as antibody 3-631-436) scFvs are analyzed to determine binding preferably using biotinylated cyclic hBNP (1-32c) to ensure the scFv format did not effect antigen binding ability and to ensure the entirety of the scFv protein is expressed on the yeast.

For example, for CN4 and CN4-related antibodies, to prevent or decrease binding to BNP, specifically hBNP and a hBNP fragment, the VH and VL segments of the preferred VH/VL pair(s) can be randomly mutated, preferably within the CDR2 region of VH, and the CDR2 region of VL. To select for scFv that prevent binding to BNP, a portion of each CDR and/or framework region can be replaced with a degenerate single-stranded oligonucleotide encoding mutations within three amino acids codon lengths within the CDR being targeted. The replacement of a portion of each CDR with a new randomized sequence (up to 8000 possibilities) can be accomplished by homologous recombination in yeast (See, e.g. Example 1). These randomly mutated VH, VL and/or framework segments can be analyzed on for example, a flow cytometer for a lack of binding to hBNP or hBNP fragment in the context of an scFv; scFvs exhibiting little to no fluorescence due to no binding to labeled BNP antigen can then be isolated and the CDR mutation identified by sequencing. The mutations from the VH, VL and/or framework region chains can then be combined into a single entity and analyzed for binding to BNP antigen.

Following screening of a recombinant scFv display library, clones having the desired characteristics are selected for conversion. Nucleic acid molecules encoding the selected antibody can be recovered from the display package (e.g., from the yeast expression vector) and subcloned into other expression vectors by standard recombinant DNA techniques. If desired, the nucleic acid can be further manipulated to create other antibody forms of the disclosure (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in Section D above.

For example, for CN4 and CN4-related antibodies, to prevent or decrease any binding affinity with BNP, the VH and VL segments of the preferred VH/VL pair(s) can be randomly mutated, preferably within the CDR2 region of VL. This can be accomplished by replacing a portion of the VL CDR with a degenerate single-stranded oligonucleotide encoding mutations for three amino acids codon lengths within the VL CDR being targeted. The replacement of a portion of the CDR with a new randomized sequence (up to 8000 possibilities) can be accomplished by homologous recombination in yeast (see, e.g. Example 1). The randomly mutated VL segments can be analyzed for a lack of binding to BNP, specifically, hBNP or hBNP fragment in the context of an scFv; on for example, a flow cytometer the scFvs exhibiting no or little fluorescence due to no binding to labeled BNP antigen when compared with an antibody produced by 3-631-436 which was deposited with the A.T.C.C. on Dec. 21, 2004 and assigned A.T.C.C. Accession No. PTA-6476 and is described in U.S. Patent Publication 2006/0183154 published on Aug. 17, 2006 can then be isolated and the CDR mutation identified by sequencing.

Following screening of a recombinant scFv display library, clones having the desired characteristics are selected for conversion. Nucleic acid molecules encoding the selected antibody can be recovered from the display package (e.g., from the yeast expression vector) and subcloned into other expression vectors by standard recombinant DNA techniques. If desired, the nucleic acid can be further manipulated to create other antibody forms of the disclosure (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in Section D above.

### F. Immunoassays

In another aspect, the present disclosure relates to immunoassays that can be used for the qualitative and/or quantitative detection of BNP or a fragment thereof, preferably hBNP or hBNP fragment, in a test sample. More specifically, the immunoassays of present disclosure employ the antibodies of the present disclosure as heterophilic blocking agents (i.e. reagents) in order to reduce heterophilic interference in said immunoassays.

The immunoassays of the present disclosure can be conducted using any format known in the art, such as, but not limited to, a sandwich format, a competitive inhibition format (including both forward or reverse competitive inhibition assays) or in a fluorescence polarization format.

In immunoassays for the qualitative detection of hBNP or hBNP fragment in a test sample, at least one antibody that binds to certain epitopes of hBNP or a hBNP fragment thereof is contacted with at least one test sample suspected of containing or that is known to contain hBNP or hBNP fragment to form an antibody-hBNP immune complex. Any antibodies known to bind to hBNP or a hBNP fragment known in the art can be used. For example, the antibodies described in U.S. Patent Publication No. US 2008/0124811, a monoclonal antibody is produced by hybridoma cell line 106.3 (A.T.C.C. Accession No. HB-12044) or a monoclonal antibody produced by hybridoma cell line 201.3 (A.T.C.C. Accession No. HB 12045), can be used in such immunoassays to form such antibody-hBNP immune complexes in at least one test sample. These immune complexes can then detected using routine techniques known to those skilled in the art. For example, the antibody of the present disclosure can be labeled with a detectable label to detect the presence of an antibody-hBNP complex. Alternatively, the hBNP or hBNP fragments in the test sample can be labeled with a detectable label and the resulting antibody-hBNP immune complexes detected using routine techniques known to those skilled in the art. Detectable labels and their attachment to antibodies are discussed in more detail infra.

Alternatively, a second antibody that binds to the hBNP or hBNP fragment and that contains a detectable label can be added to the test sample and used to detect the presence of the antibody-hBNP complex. Any detectable label known in the art can be used. Detectable labels and their attachment to antibodies are discussed in more detail infra.

In immunoassays for the quantitative detection of hBNP, such as a sandwich type format, at least two antibodies are employed to separate and quantify hBNP or hBNP fragment in a test sample. More specifically, the immunoassay with at least two antibodies bind to certain epitopes of hBNP or hBNP fragment forming an immune complex which is referred to as a “sandwich”. Generally, one or more antibodies can be used to capture the hBNP or hBNP fragment in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, it is preferred that both antibodies binding to their epitope are not diminished by the binding of any other antibody in the assay to its respective epitope. In other words, antibodies should be selected so that the one or more first antibodies brought into contact with a test sample suspected of containing hBNP or hBNP fragment do not bind to all or part of an epitope recognized by the second or subsequent antibodies, thereby interfering with the ability of the one or more second detection antibodies to bind to the hBNP or hBNP fragment. In a sandwich assay, the antibodies of the present disclosure can be added directly to the test sample or can be added simultaneously or sequentially with any other antibody or reagents added at anytime during the immunoassay.

In a preferred embodiment, the test sample suspected of containing hBNP or a hBNP fragment can be contacted with at least one first capture antibody (or antibodies) and at least one second detection antibodies either simultaneously or sequentially. In the sandwich assay format, a test sample suspected of containing hBNP or hBNP fragment is first brought into contact with at least one first capture antibody that specifically binds to a particular epitope under conditions which allow the formation of a first antibody-hBNP complex. If more than one capture antibody is used, a first multiple capture antibody-hBNP complex is formed. In a sandwich assay, the antibodies, preferably, at least one capture antibody, are used in molar excess amounts of the maximum amount of hBNP or hBNP fragment expected in the test sample. For example, from about 5 μg/mL to about 1 mg/mL of antibody per mL of microparticle coating buffer can be used. The antibodies of the present disclosure can be added directly to the test sample or can be added simultaneously or sequentially with at least one first capture antibody, with at least one second detection antibody or with both at least one first capture antibody and at least one second detection antibody.

Optionally, prior to contacting the test sample with at least one first capture antibody, at least one first capture antibody can be bound to a solid support which facilitates the separation of the first antibody-hBNP complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind hBNP or hBNP fragment. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample suspected of containing hBNP or an hBNP fragment is brought into contact with the at least one first capture antibody, the test sample is incubated in order to allow for the formation of a first capture antibody (or multiple antibody)-hBNP complex. The incubation can be carried out at a pH of from about 4.5 to about 10.0, at a temperature of from about 2° C. to about 45° C., and for a period from at least about one (1) minute to about eighteen (18) hours, preferably from about 2-6 minutes, most preferably from about 3-4 minutes.

After formation of the first (or multiple) capture antibody-hBNP complex, the complex is then contacted with at least one second detection antibody (under conditions which allow for the formation of a first (or multiple) antibody-hBNP-second antibody complex). If the first antibody-hBNP complex is contacted with more than one detection antibody, then a first (or multiple) capture antibody-hBNP-multiple antibody detection complex is formed. As with first antibody, when at least second (and subsequent) antibody is brought into contact with the first antibody-hBNP complex, a period of incubation under conditions similar to those described above is required for the formation of the first (or multiple) antibody-hBNP-second/multiple antibody complex. Preferably, at least one second antibody contains a detectable label. The detectable label can be bound to at least one second antibody prior to, simultaneously with or after the formation of the first (or multiple) antibody-hBNP-second/multiple antibody complex. Any detectable label known in the art can be used. For example, the detectable label can be a radioactive label, such as, ³H, ¹²⁵I, ³⁵S, ¹⁴C, ³²P, ³³P, an enzymatic label, such as horseradish peroxidase, alkaline peroxidase, glucose 6-phosphate dehydrogenase, etc., a chemiluminescent label, such as, acridinium esters, luminal, isoluminol, thioesters, sulfonamides, phenanthridinium esters, etc. a fluorescence label, such as, fluorescein (5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

The detectable label can be bound to the antibodies either directly or through a coupling agent. An example of a coupling agent that can be used is EDAC (1-ethyl-3-(3-dimethylaminopropyl) carbodiimide, hydrochloride) that is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

The first antibody/multiple-hBNP-second/multiple antibody complex can be, but does not have to be, separated from the remainder of the test sample prior to quantification of the label. For example, if at least first capture antibody is bound to a solid support, such as a well or a bead, separation can be accomplished by removing the fluid (from the test sample) from contact with the solid support. Alternatively, if at least first capture antibody is bound to a solid support it can be simultaneously contacted with the hBNP-containing sample and the at least one second detection antibody to form a first (multiple) antibody-hBNP-second (multiple) antibody complex, followed by removal of the fluid (test sample) from contact with the solid support. If at least first capture antibody is not bound to a solid support, then the first antibody/multiple-hBNP-second/multiple antibody complex does not have to be removed from the test sample for quantification of the amount of the label.

After formation of the labeled first antibody-hBNP-second antibody complex, the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. Once the amount of the label in the complex has been quantified, the concentration of hBNP or hBNP fragment in the test sample is determined by use of a standard curve that has been generated using serial dilutions of hBNP or hBNP fragment of known concentration. Other than using serial dilutions of hBNP or hBNP fragment, the standard curve can be generated gravimetrically, by mass spectroscopy and by other techniques known in the art.

In a forward competitive format, an aliquot of labeled hBNP, hBNP fragment or hBNP analogue thereof of a known concentration is used to compete with hBNP or hBNP fragment in a test sample for binding to hBNP antibody. Peptides of hBNP, hBNP fragments and hBNP analogues thereof and methods of making peptides of hBNP, hBNP fragments and hBNP analogues are known in the art (See, for example, U.S. Pat. No. 6,162,902). Optionally, the antibodies of the present disclosure can be added to the test sample or to the aliquot containing the labeled hBNP, hBNP fragment or hBNP analogue.

In a forward competition assay, an immobilized antibody (such as an antibody of the present disclosure) can either be sequentially or simultaneously contacted with the test sample and a labeled hBNP, hBNP fragment or hBNP analogue thereof. Optionally, the antibodies of the present disclosure can be added directly to the test sample or can be sequentially or simultaneously contacted with the test sample and the labeled hBNP, hBNP fragment or hBNP analogue. The hBNP peptide, hBNP fragment or hBNP analogue can be labeled with any detectable label known to those skilled in the art, including those detectable labels discussed above in connection with the sandwich assay format. In this assay, the antibody can be immobilized on to a solid support using the techniques discussed previously herein. Alternatively, the antibody can be coupled to an antibody, such as an antispecies antibody, that has been immobilized on to a solid support, such as a microparticle.

The labeled hBNP peptide, hBNP fragment or hBNP analogue, the test sample and the antibodies are incubated under conditions similar to those described above in connection with the sandwich assay format. Two different species of antibody-hBNP complexes are then generated. Specifically, one of the antibody-hBNP complexes generated contains a detectable label while the other antibody-hBNP complex does not contain a detectable label. The antibody-hBNP complex can be, but does not have to be, separated from the remainder of the test sample prior to quantification of the detectable label. Regardless of whether the antibody-hBNP complex is separated from the remainder of the test sample, the amount of detectable label in the antibody-hBNP complex is then quantified. The concentration of hBNP or hBNP fragment in the test sample can then be determined by comparing the quantity of detectable label in the antibody-hBNP complex to a standard curve. The standard curve can be generated using serial dilutions of hBNP or hBNP fragment of known concentration, by mass spectroscopy, gravimetrically and by other techniques known in the art.

The antibody-hBNP complex can be separated from the test sample by binding the antibody to a solid support, such as the solid supports discussed above in connection with the sandwich assay format, and then removing the remainder of the test sample from contact with the solid support.

The labeled hBNP (or hBNP fragment or hBNP analogue thereof) that is used to compete with hBNP or a hBNP fragment in the test sample for binding to the antibody can be intact hBNP 1-32, any hBNP fragment thereof or any hBNP analogue provided that said hBNP peptide, hBNP fragment or hBNP analogue contains a sequence of amino acids that corresponds to an epitope that is recognized by the antibody.

In a reverse competition assay, an immobilized hBNP peptide, hBNP fragment or hBNP analogue thereof can either be sequentially or simultaneously contacted with a test sample and at least one labeled antibody. The antibodies of the present disclosure can be included in the test sample. Optionally, the immobilized hBNP peptide, hBNP fragment or hBNP analogue can either be sequentially or simultaneously contacted with the test sample, at least one labeled antibody and at least one antibody of the present disclosure.

The hBNP peptide, hBNP fragment or hBNP analogue can be bound to a solid support, such as the solid supports discussed above in connection with the sandwich assay format.

The immobilized hBNP peptide, hBNP peptide fragment or hBNP analogue thereof, test sample, at least one labeled antibody and at least one antibody of the present disclosure) are incubated under conditions similar to those described above in connection with the sandwich assay format. Two different species hBNP-antibody complexes are then generated. Specifically, one of the hBNP-antibody complexes generated is immobilized and contains a detectable label while the other hBNP-antibody complex is not immobilized and contains a detectable label. The non-immobilized hBNP-antibody complex and the remainder of the test sample are removed from the presence of the immobilized hBNP-antibody complex through techniques known in the art, such as washing. Once the non-immobilized hBNP antibody complex is removed, the amount of detectable label in the immobilized hBNP-antibody complex is then quantified. The concentration of hBNP or hBNP fragment in the test sample can then be determined by comparing the quantity of detectable label in the hBNP-complex to a standard curve. The standard curve can be generated using serial dilutions of hBNP or hBNP fragment of known concentration, by mass spectroscopy, gravimetrically and by other techniques known in the art.

In a fluorescence polarization assay, in one embodiment, a first antibody or functionally active fragment thereof is first contacted with an unlabeled test sample suspected of containing hBNP or a hBNP fragment thereof to form an unlabeled hBNP-antibody complex. The unlabeled hBNP-first antibody complex is then contacted with a fluorescently labeled hBNP, hBNP fragment or hBNP analogue thereof. The labeled hBNP, hBNP fragment or hBNP analogue competes with any unlabeled hBNP or hBNP fragment in the test sample for binding to the antibody or functionally active fragment thereof. The amount of labeled hBNP-first antibody complex formed is determined and the amount of hBNP in the test sample determined via use of a standard curve. Optionally, the antibodies of the present disclosure (which would be a second antibody) can be included in the test sample. Alternatively, the antibodies of the present disclosure can, along with the first antibody, be simultaneously or sequentially contacted with the unlabeled test sample suspected of containing hBNP or a hBNP fragment.

The antibody, labeled hBNP peptide, hBNP peptide fragment or hBNP analogue thereof and test sample and at least one labeled antibody are incubated under conditions similar to those described above in connection with the sandwich assay format.

### F. Immunoassay Kits

The present disclosure also contemplates kits for detecting the presence of BNP, preferably, human BNP or any hBNP fragment, in a test sample. Such kits can comprise one or more of the negative mimic antibodies or NEMIC antibodies described herein (e.g., Section B). More specifically, if the kit is a kit for performing an immunoassay, the kit optionally can comprise the negative mimic antibodies or NEMIC antibodies described herein and instructions for performing the immunoassay. Optionally, the kit can further comprise one or more additional antibodies that can be used as capture antibodies and/or detection antibodies.

Thus, the present disclosure further provides for diagnostic and quality control kits comprising one or more negative mimic antibodies or NEMIC antibodies of the disclosure. Optionally the assays, kits and kit components of the disclosure are optimized for use on commercial platforms (e.g., immunoassays on the Prism®, AxSYM®, ARCHITECT® and EIA (Bead) platforms of Abbott Laboratories, Abbott Park, Ill., as well as other commercial and/or in vitro diagnostic assays). Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories, Abbott Park, Ill.) electrochemical immunoassay system that performs sandwich immunoassays for several cardiac markers, including TnI, CKMB and BNP. Immunosensors and methods of operating them in single-use test devices are described, for example, in US Patent Applications 20030170881, 20040018577, 20050054078 and 20060160164 which are incorporated herein by reference. Additional background on the manufacture of electrochemical and other types of immunosensors is found in U.S. Pat. No. 5,063,081 which is also incorporated by reference for its teachings regarding same.

Optionally the kits include quality control reagents (e.g., sensitivity panels, calibrators, and positive controls). Preparation of quality control reagents is well known in the art, and is described, e.g., on a variety of immunodiagnostic product insert sheets. BNP sensitivity panel members optionally can be prepared in varying amounts containing, e.g., known quantities of BNP antibody ranging from “low” to “high”, e.g., by spiking known quantities of the BNP antibodies according to the disclosure into an appropriate assay buffer (e.g., a phosphate buffer). These sensitivity panel members optionally are used to establish assay performance characteristics, and further optionally are useful indicators of the integrity of the immunoassay kit reagents, and the standardization of assays.

The antibodies provided in the kit can incorporate a detectable label, such as a fluorophore, radioactive moiety, enzyme, biotin/avidin label, chromophore, chemiluminescent label, or the like, or the kit may include reagents for labeling the antibodies or reagents for detecting the antibodies (e.g., detection antibodies) and/or for labeling the antigens or reagents for detecting the antigen. The antibodies, calibrators and/or controls can be provided in separate containers or pre-dispensed into an appropriate assay format, for example, into microtiter plates.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

The kit further can optionally include instructions for use, which may be provided in paper form or in computer-readable form, such as a disc, CD, DVD or the like.

### G. Adaptation of Kits

The kit (or components thereof), as well as the method of determining the detecting the presence or concentration of BNP, preferably, human BNP or any hBNP fragment in a test sample by an assay using the components and methods described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., BNP capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours) an automated or semi-automated format (e.g., ARCHITECT®, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an BNP, preferably a human BNP or hBNP fragment assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the second detection antibody labeled with alkaline phosphatase (or other label). Within the fluid pouch of the cartridge is an aqueous reagent that includes p-aminophenol phosphate.

In operation, a sample suspected of containing BNP, preferably a human BNP or hBNP fragment is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between BNP, preferably human BNP, BNP capture antibody, and the labeled detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of BNP, preferably human BNP in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent.

By way of example and not of limitation, examples of the present disclosure shall now be given.

### Example 1

### Identification of Immunoglobulin Genes

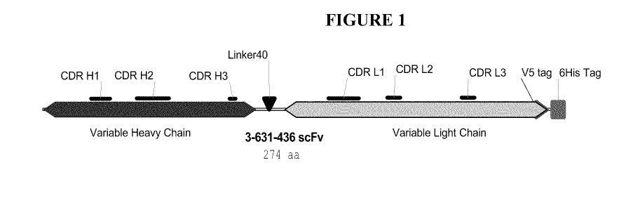

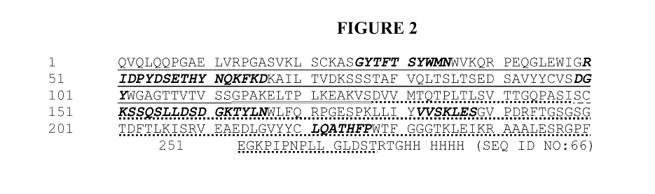

Messenger RNA was isolated from subcloned anti-BNP 3-631-436 hybridoma cells (hybridoma cell line 3-631-436 (A.T.C.C. Accession No. PTA-6476) is described in U.S. Patent Application No. 2006/0183154, the contents of which are herein incorporated by reference in their entirety). 3-631-436 mRNA was utilized in a reverse transcriptase-polymerase chain reaction using a mouse Ig primer set kit purchased from Novagen (Novagen (which is an Affiliate of Merck KGaA, Darmstadt, Germany), Cat No. 69831-3) with immunoglobulin gene specific primers contained in the kit. The resulting PCR products were sequenced and thus the immunoglobulin variable heavy chain gene was identified (See, FIGS. 1, 2 and 3A and 3B)).

Alternatively the N-terminal amino acid sequence of the 3-631-436 light chain was determined using a LC-MS-MS technique subsequently used to isolate the light chain gene. The antibody heavy and light chain were separated on a SDS-PAGE gel, trypsin digested and analyzed by LC-MS-MS. The N-terminus amino acid sequence was determined using a database search to be DVVMTQTPLTLSVTTGQPASISC (SEQ ID NO:7). A degenerate 5′ nucleotide primer (5′ GAYGTNGTNATGACNCARACNCCN 3′ where N=A, G, C, T; Y=C, T; R=A, G) (SEQ ID NO: 8) and reverse kappa chain primer supplied with the Novagen kit (above) was used to amplify the 3-631-436 kappa light chain from mRNA. The resulting PCR product was sequenced to identify the complete light chain variable gene (See, FIGS. 3A and 3B).

Cloning 3-631-436 Variable Region Genes into pYD41 Vector

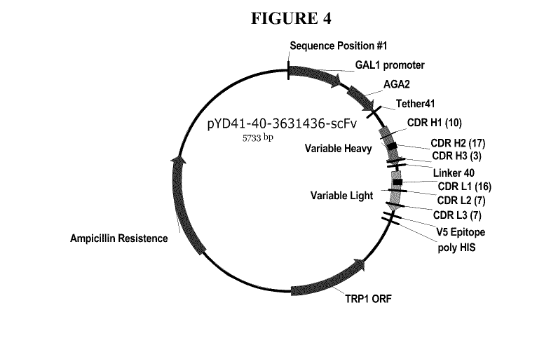

A yeast display system was used to express unmutated anti-BNP proteins (described herein infra) and a library of anti-BNP proteins on the yeast surface as a fusion to the yeast protein AGA2. A yeast display vector called pYD (Invitrogen, Carlsbad, Calif.), was used as it allows for cloning of the anti-BNP gene at the C-terminus of the AGA2 gene, a yeast mating factor (See, Boder and Wittrup, Nature Biotechnology, 15:553-557 (June 1997). Other critical features of the pYD vector include a galactose inducible promoter and an epitope tag, V5, on the C-terminus of the inserted anti-BNP gene (See, FIGS. 3A-3B and 4).

The yeast display platform utilizes an antibody format known as the single-chain variable fragment. In the scFv format, the variable heavy domain is connected to the variable light domain through a flexible linker (variable heavy domain—Linker GPAKELTPLKEAKVS (SEQ ID NO:9)—variable light domain).

PCR single overlap extension (SOE) was used to combine the variable heavy (VH) and the variable light genes (VL) for the 3-631-436 scFv construct (See, FIG. 1 and FIGS. 2A and 3B). The 3-631-436 scFv DNA was cloned into the yeast display vector pYD41 using vector restriction sites SfiI and NotI. The pYD41-3-631-436 scFv vector was transformed into DH5α E. coli. Plasmid DNA was then isolated from the E. coli and the 3-631-436 scFv insert was sequenced to ensure the scFv was cloned in frame with the AGA2 protein.

The cloning site for the scFv into the yeast display vector pYD41 is in an ORF that includes the following genes: AGA2-tether linker 41-X press epitope tag-3-631-436 variable heavy chain-Linker 40-3-631-436 variable light chain-V5 epitope tag—Six His tag. In addition, the yeast strain EBY100 is a tryptophan auxotroph and the pYD41 vector encodes for tryptophan as the system's selectable marker.

Transformation into Saccharomyces cerevisiae Strain EBY100

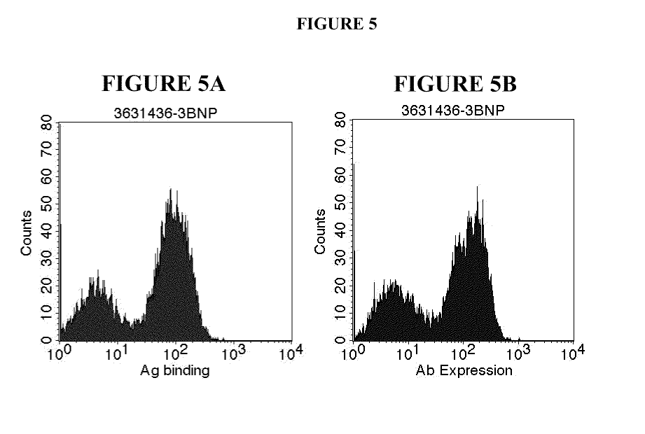

Yeast display plasmid, pYD41-3-631-436 scFv, was transformed into S. cerevisiae EBY100 using Gietz and Schiestl Method (See, Schiestl and Gietz, Current Genetics, 16(5-6):339-46 (December 1989)). Dilutions of the transformation reaction were plated on selective glucose plates (2% glucose (0.67% yeast nitrogen base, 0.105% HSM-trp-ura, 1.8% bacterial agar, 18.2% sorbitol, 0.86% NaH₂PO₄H₂O, 1.02% Na₂HPO₄ 7H₂O)) and incubated at 30° C. for 48-72 hours. Selective glucose media was inoculated with individual colonies and grown shaking at 30° C. for 16-20 hours. Protein expression was induced in colonies by transferring 0.5 OD600 of cells/ml (1e7 cells/0.5 OD/ml) to selective galactose media. Colonies were shaken at 20° C. for 16-24 hours and then analyzed by the FACS Aria flow cytometer for binding to cyclic BNP (referred to as “1-32c”) (SEQ ID NO:10) and anti-V5. For flow cytometry assays, yeast cells expressing 3-631-436 scFv were incubated with biotinylated: cyclic BNP (1-32c) (SEQ ID NO:10) or anti-V5 antibody followed by streptavidin: phycoerythrin (SA:PE, BD Pharmingen) or FITC labeled goat anti-mouse immunoglobulin-(GAM:FITC, Molecular Probes (which is an Affiliate of Invitrogen, Carlsbad, Calif.)). The flow cytometry histograms as shown in FIGS. 5A and 5B illustrate full-length surface expression of 3-631-436 scFv (anti-V5 binding) and binding of 3-631-436 scFv to cyclic BNP (1-32c) (SEQ ID NO:10).

### Generation of 3-631-436 CDR Directed Libraries

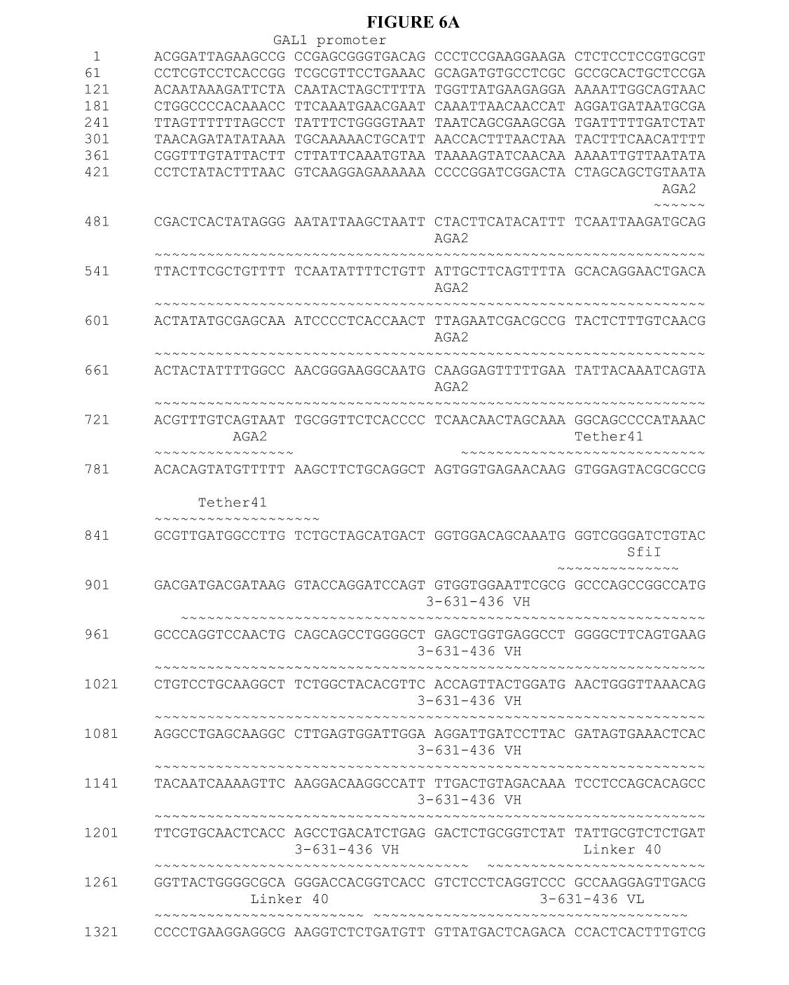

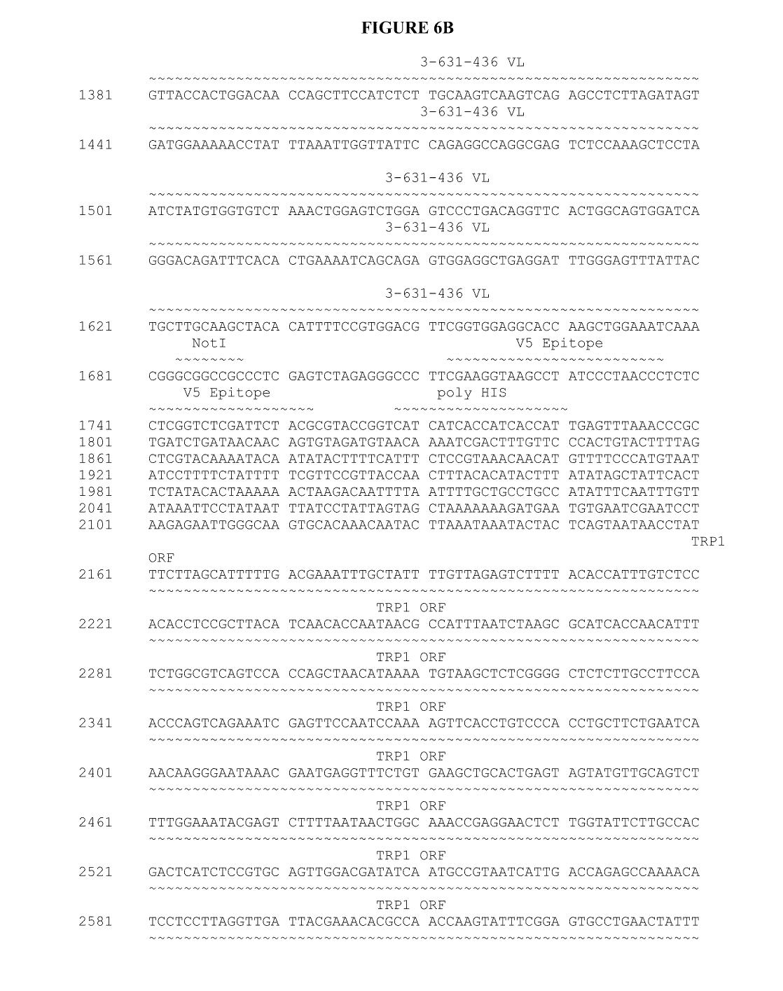

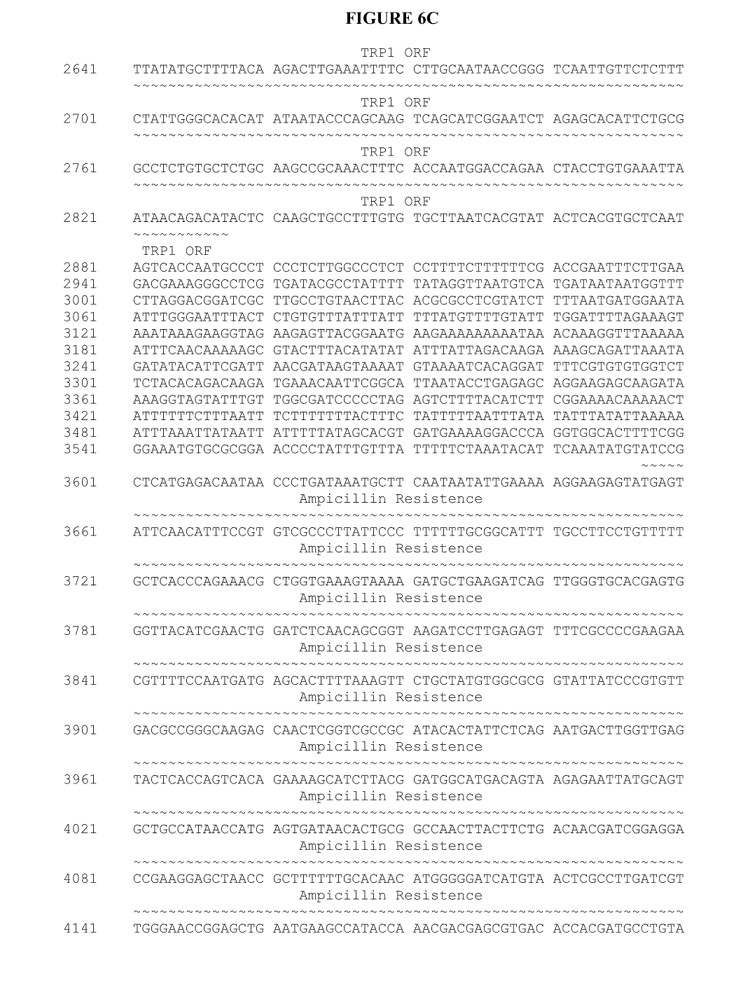

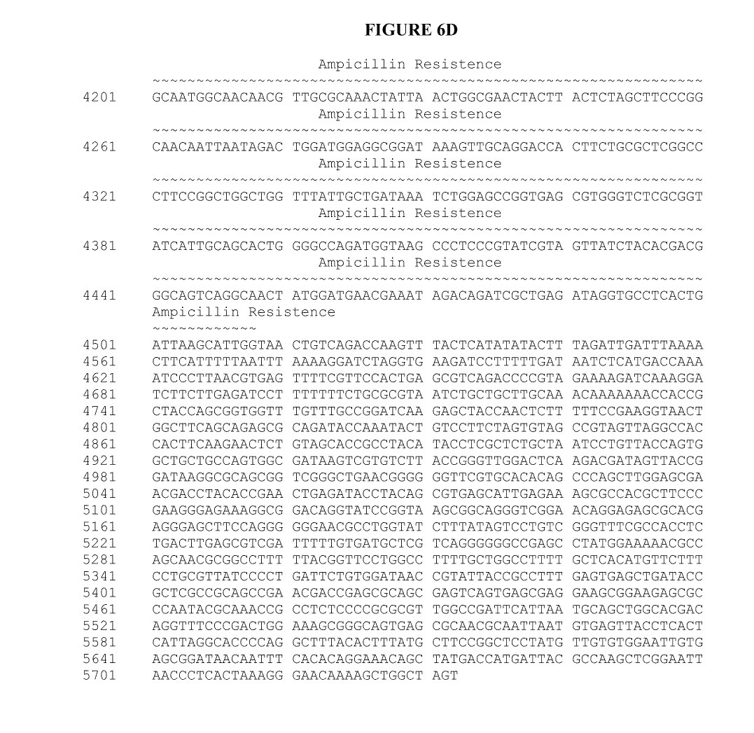

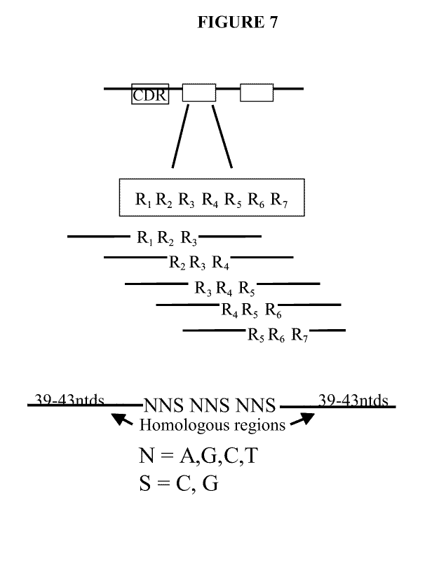

Mutagenesis was directed to the three heavy and three light chain complementary determining regions (CDR) of antibody 3-631-436 (See, FIGS. 3A-3B and 6A and 6D and SEQ ID NOS:11-13) since these loops are the major antigen contact sites. CDR loop lengths and numbering were defined using Kabat and Oxford Molecular's AbM modeling software. Individual libraries were composed that randomly mutated three amino acid positions of the CDR in a single library with the mutagenic window shifted by one amino acid per library (See, FIG. 7). The library diversity for an individual library totaled 203 or 8,000 possible variants with every amino acid sampled at every CDR position. For 3-631-436 scFv, a total of 50 libraries were generated 24 variable heavy and 26 variable light libraries.

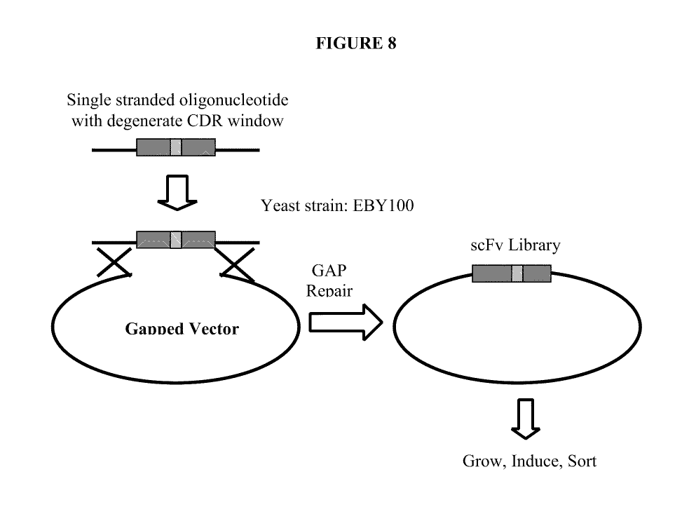

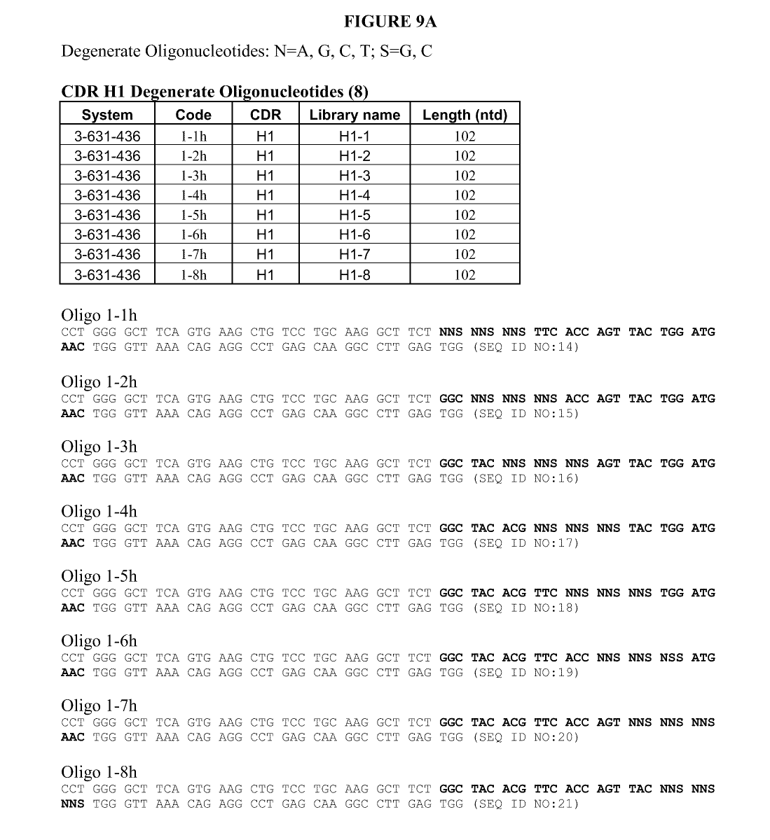

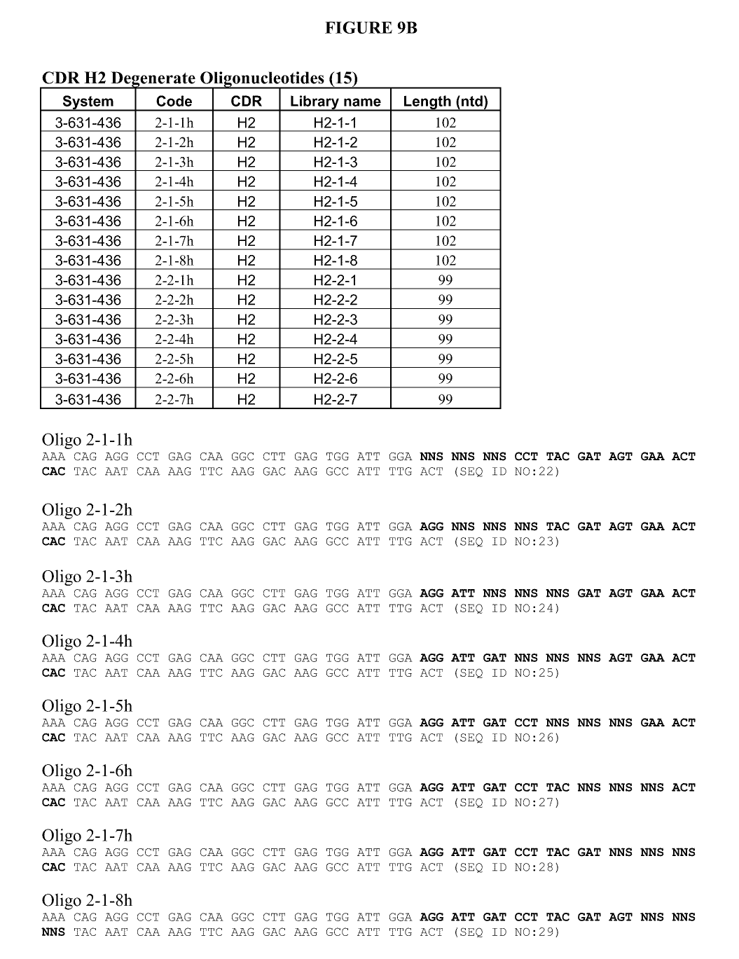

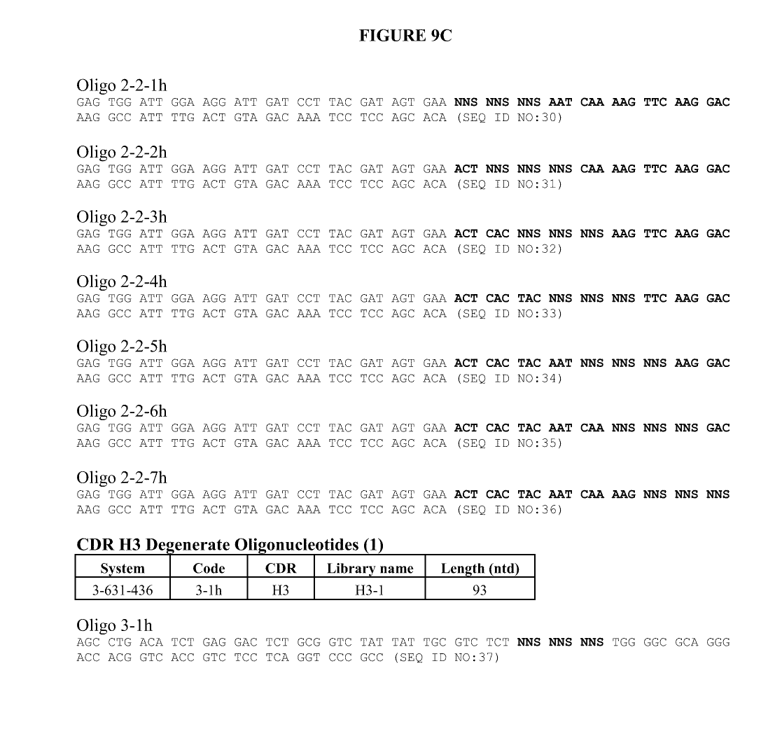

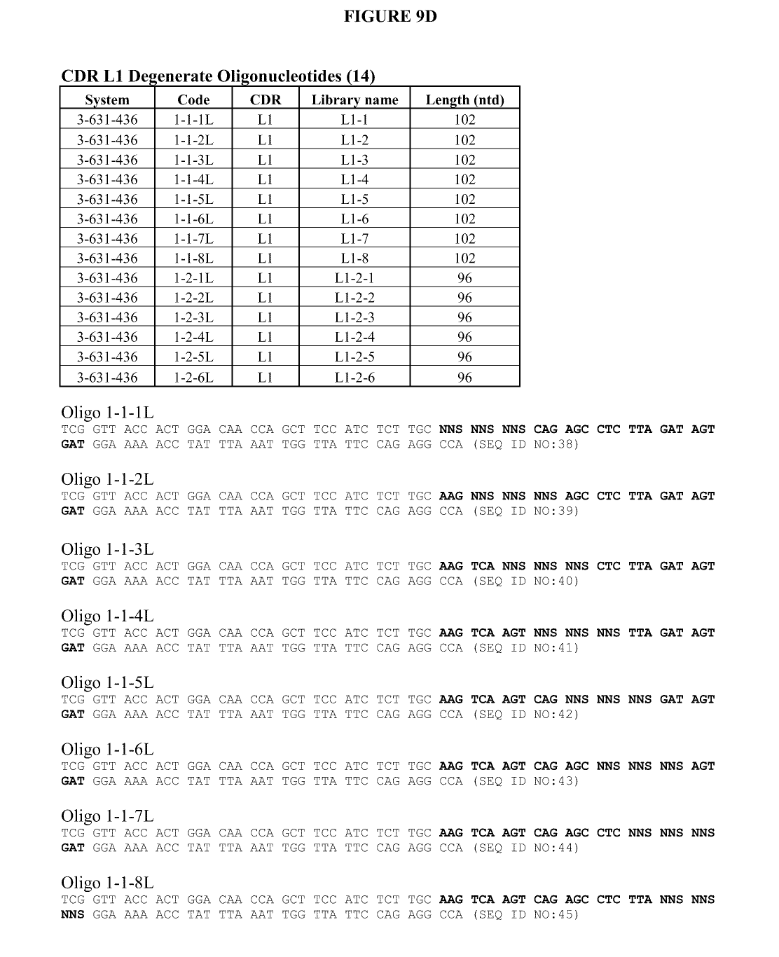

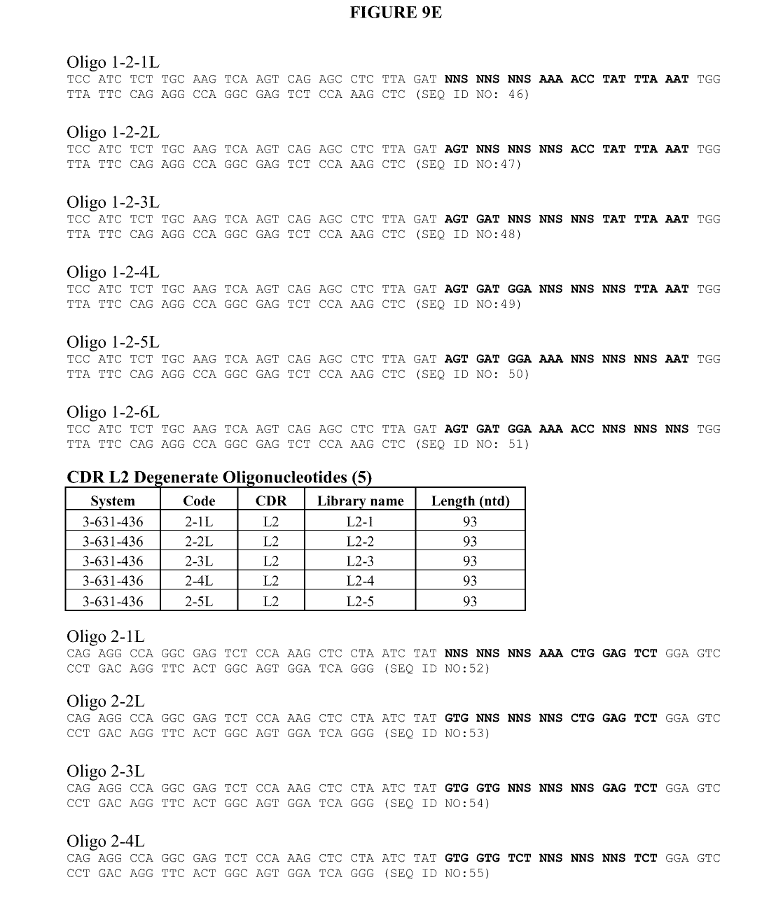

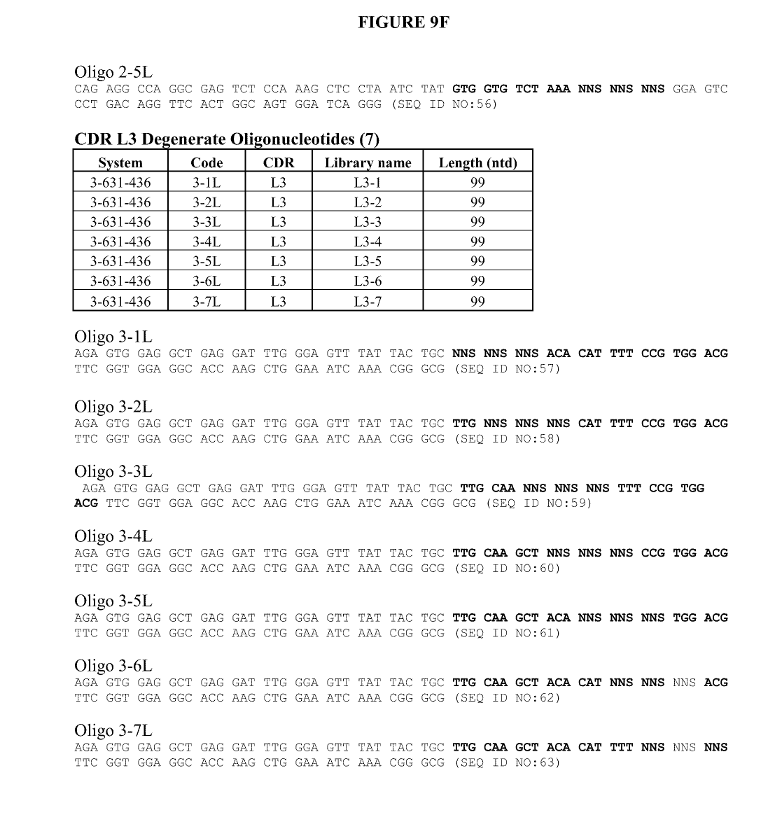

Libraries were generated by combining linearized gapped pYD41-3-631-436 vector and single stranded oligonucleotides with chemically competent EBY100 yeast (See, FIG. 8). The gapped pYD41 vector is a vector created by PCR that lacks a specific region of each CDR that is replaced in library construction by the single stranded degenerate oligonucleotide. Degenerate single-stranded oligonucleotides are 90-105 nucleotides long with 39-43 nucleotides of homology to the pYD41-3-631-436 scFv vector on each side of the nine degenerate nucleotide window. The oligonucleotides for each library, 50 total, were synthesized (See, FIGS. 9A-9F and SEQ ID NOS: 14-63). Gapped vector (1 ug) and the degenerate oligonucleotide (16 ug) were combined with EBY100 yeast (3e8 cells) and transformed using the Gietz and Schiestl library transformation protocol (Schiestl and Gietz, Current Genetics, 16(5-6):339-46 (December 1989)). The degenerate oligonucleotide and the pYD41-3-631-436 scFv gapped vector cyclize during transformation due to homologous recombination facilitated by the nucleotide overlap and the mechanism of yeast endogenous gap repair. Libraries were grown at 30° C. for 48-72 hours in selective glucose media and passed again in selective glucose media. Individual libraries were pooled according to their variable chain such as VL library or VH library prior to induction of protein expression for library sorting.

### 3-631-436 Mutagenic CDR Library Sorting

3-631-436 libraries were sorted based on a loss of signal sorting strategy. 3-631-436 CDR mutagenic libraries were induced in galactose expression media at 20° C. for 18-24 hours. At room temperature, 3-631-436 mutagenic libraries were washed with PBS/1% BSA blocking buffer and incubated with 330 nM of biotinylated cyclic BNP (1-32c) and anti-V5 antibody at 2.5 ug/mL. After 30-60 minutes, mutagenic libraries were washed twice and incubated on ice with SA-PE (1:200 dilution) and GAM-IgG2a AlexaFluor 488 (1:100 dilution) for 30-45 minutes. Finally, cells were washed, analyzed and sorted on the FACS Aria. Sort gate was set based on unmutated 3-631-436 binding under the same assay conditions. The sort gate was set to isolate low PE and high FITC fluorescence cells representing those full-length clones that do not bind human BNP. Sorted cells were grown in selective glucose media and grown 18-24 hours at 30° C. Sort 1 cells were induced and sorting was repeated for one additional round.

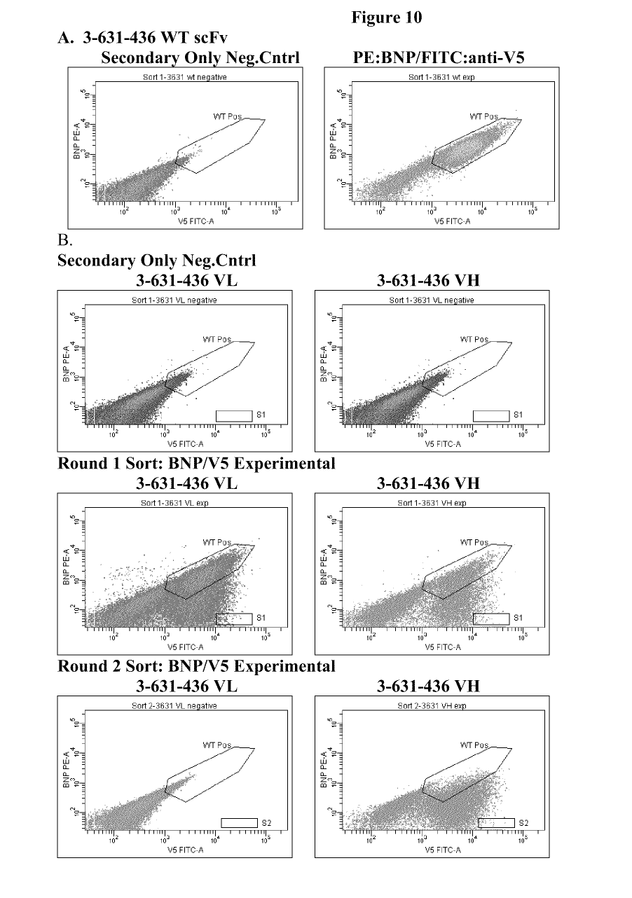

After the last sort, sorted cells were plated onto selective glucose plates and placed at 30° C. for ˜72 hours. Both the VH and VL libraries sorted 2 times exhibited low human BNP binding and high surface expression levels (FIG. 10). Individual yeast colonies from these libraries were inoculated in selective glucose media, cryopreserved and induced in selective galactose media. Individual colonies were then characterized in the same flow cytometry method as described for library sorting.

### Analysis of Selected 3-631-436 Loss of Binding Variants

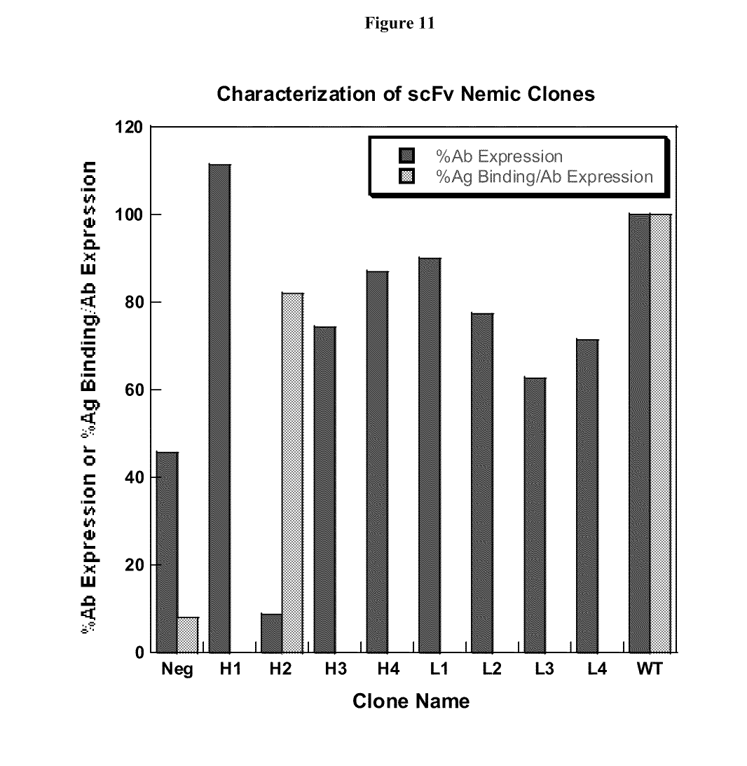

3-631-436 libraries were characterized similar to the cell sorting technique. Four scFv clones from each sorted library designated H1, H2, H3, H4, L1, L2, L3 and L4, a negative control and the 3-631-436 WT clone were characterized for human BNP and anti-V5 binding. At room temperature, 3-631-436 loss variants were washed with blocking buffer and incubated with 83 nM of biotinylated cyclic BNP (1-32c) and anti-V5 antibody at 2.5 ug/mL. After 35 minutes, cells were washed and incubated on ice with SA-PE (1:200 dilution) and GAM-IgG2a AlexaFluor 488 (1:100 dilution) for 45 minutes. Finally, cells were washed and analyzed on the FACS Aria. % Antibody expression was calculated as follows: MFU for V5 binding for Clone x/MFU for V5 binding for WT*100. % Antigen binding was calculated as follows: MFU for human BNP binding for Clone x/MFU for human BNP binding for WT*100. As depicted in FIG. 11, H1, H3, H4, L1, L2, L3 and L4 did not bind human BNP at 83 nM.

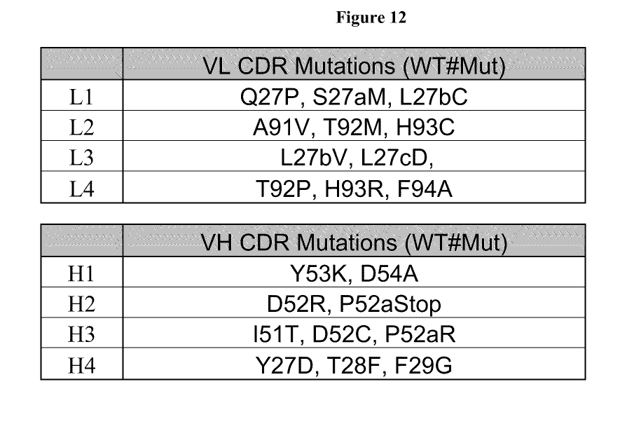

Selected 3-631-436 scFv variants were sequenced to determine clonal diversity. Initially, a scFv PCR product was amplified by yeast colony PCR reaction. SD culture was boiled in the presence of 0.1% SDS followed by a PCR reaction including 0.10% Triton X100. Pure plasmid DNA was then sequenced using pYD41 vector specific primers (pYD41 for -TAGCATGACTGGTGGACAGC (SEQ ID NO:64) and pYD41rev-CGTAGAATCGAGACCGAG (SEQ ID NO: 65)). Nucleotide and amino acid sequence data for 3-631-436 scFv variants is shown in FIG. 12. Position numbers refers to amino acid position given by numbering using the Kabat numbering system.

### Generation of 3-631-436 Combined Loss Variant Library

An additional library was generated that combined the variable heavy chain and variable light chain mutants from the initial sorting approach defined above. Using the round two SD culture sorted cells, yeast colony PCR was utilized to amplify the pool of VH or VL genes from the VH loss variant library or the VL loss variant library respectively. Libraries were generated by combining digested pYD41 vector and VL and VH PCR products with chemically competent EBY100 yeast. The pYD41 vector was digested with SfiI/NotI and dephosphorylated with calf intestine phosphatase. Digested vector (100 ng) and VH and VL PCR products (5:1 molar ratio insert:vector) were combined with EBY100 yeast (1e8 cells) and transformed using the Gietz and Schiestl library transformation protocol (Schiestl and Gietz, Current Genetics, 16(5-6):339-46 (December 1989)). The PCR products and the pYD41 gapped vector cyclize during transformation due to homologous recombination facilitated by the nucleotide overlap and the mechanism of yeast endogenous gap repair. Libraries were grown at 30° C. for 48-72 hours in selective glucose media and passed again in selective glucose media.

### 3-631-436 Combined Loss Variant Library Sorting

3-631-436 libraries were sorted under more stringent conditions than those defined above for the loss of signal sorting strategy. 3-631-436 CDR mutagenic libraries were induced in galactose expression media at 20° C. for 18-24 hours. At room temperature, 3-631-436 mutagenic libraries were washed with PBS/1% BSA blocking buffer and incubated with biotinylated cyclic BNP (1-32c) at 1 uM for sort 1 and 2 uM for sort 2 and anti-V5 antibody at 2.5 ug/mL. After 30-45 minutes, mutagenic libraries were washed twice and incubated on ice with SA-PE (1:200 dilution) and GAM-IgG2a AlexaFluor 488 (1:200 dilution) for 30-45 minutes. Finally, cells were washed, analyzed and sorted on the FACS Aria. Sort gate was set based on unmutated 3-631-436 binding under the same assay conditions. The sort gate was set to isolate low PE and high FITC fluorescence cells representing those full-length clones that do not bind human BNP. Sorted cells were grown in selective glucose media and grown 18-24 hours at 30° C. Sort 1 cells were induced and sorting was repeated for one additional round.

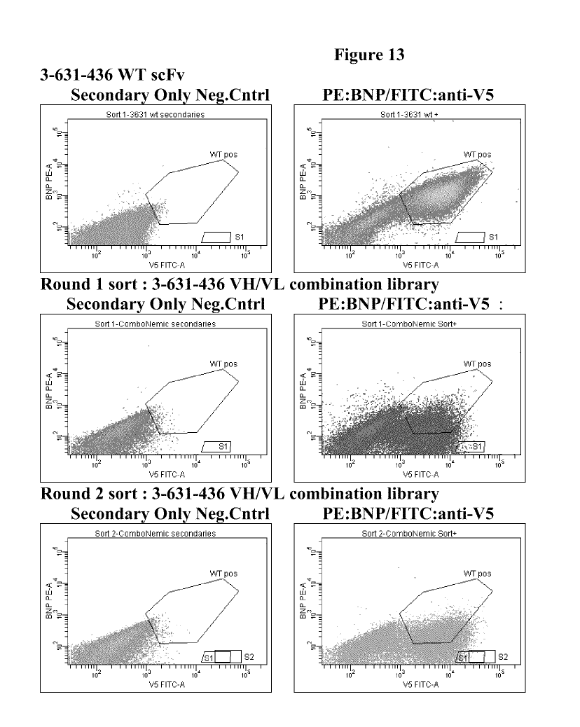

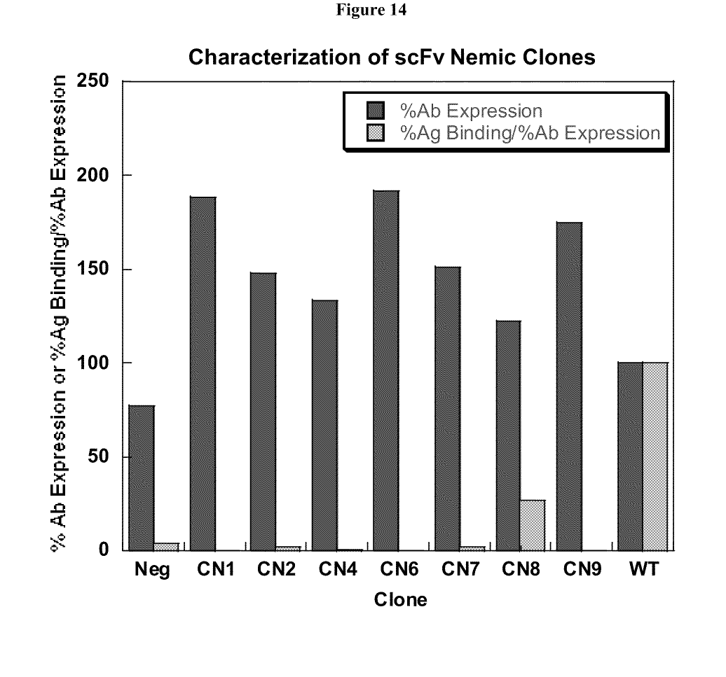

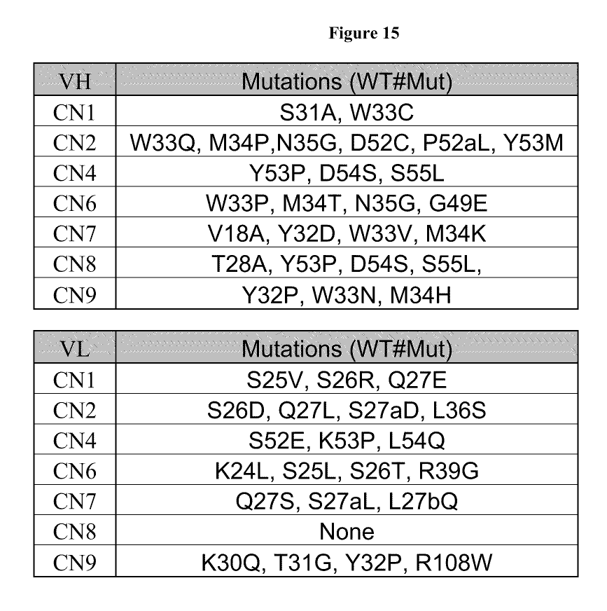

After the last sort, sorted cells were plated onto selective glucose plates and placed at 30° C. for ˜72 hours. The combined loss variant library exhibited low human BNP binding and high surface expression levels (FIG. 13). Individual yeast colonies from these libraries were inoculated in selective glucose media, cryopreserved and induced in selective galactose media. Individual combined loss variants or negative mimics were characterized as previously described at 2 uM human BNP (FIG. 14). Of the 10 clones analyzed CN3, CN5, and CN10 exhibited no binding by the anti-V5/FITC goat-anti-mouse reagent and were not further pursued since they were not full length proteins. Clones CN1, CN2, CN4, CN6, CN7, CN8, and CN9 were identified as loss variants and sequenced as previously described (FIG. 15). CN8 did exhibit weak antigen binding relative to the WT clone so the mutations for CN8 did not completely inhibit BNP binding.

### Cloning and Soluble Expression of 3-631-436 Chimeric Antibodies in a Transient or Stable Expression System

Selected 3-631-436 variant CN4, was converted to chimeric mouse IgG_{2b}/kappa antibodies through cloning of the 3-631-436 variable domain into the transient expression vector system called pBOS (Abbott Bioresearch Center, Worcester, Mass.). More specifically, PCR was used to amplify the variable heavy and variable light chain genes with restriction sites for cloning into separate pBOS vectors (Mizushima and Nagata, Nucleic Acids Research, 18:5322, (1990)). The variable heavy and variable light genes were ligated in digested/dephosphorylated vector and transformed into DH5α E. coli. Plasmid DNA was purified from E. coli and transfected into 293H cells using lipofectamine (Invitrogen, Carlsbad, Calif.). Transient antibody was expressed for the 3-631-436 CN4 variants.

Using the pBOS-3-631-436 CN4 heavy and light vectors, a stable CHO cell line plasmid was created in a two-step cloning procedure. First, variable heavy chain and variable light genes were ligated in frame to the mouse constant genes in pBV and pJV plasmids (Abbott Bioresearch Center, Worcester, Mass.), respectively, using the restriction enzymes SrfI/NotI. Ligation reactions were transformed into DH5α E. coli and plasmid DNA was subsequently isolated from individual colonies. The pBV-3-631-436 CN4 mouse variable heavy-IgG2b and pJV-3-631-436 CN4 mouse variable light-kappa were sequenced at the cloning sites.

The second cloning step involved combining the heavy chain IgG2b genes and the light chain kappa genes into a single stable cell line vector. The pBV-3-631-436 CN4 and pJV-3-631-436 CN4 vectors were digested with AscI/PacI. The VL-mouse kappa constant and the VH-mouse IgG2b constant DNA fragments were gel purified and ligated to produce the stable cell line vector called pBJ-3-631-436 CN4. The pBJ-3-631-436 CN4 heavy/light chimeric plasmid was transformed into CHO cells using calcium phosphate protocol.

### Example 2

### KinExA Characterization

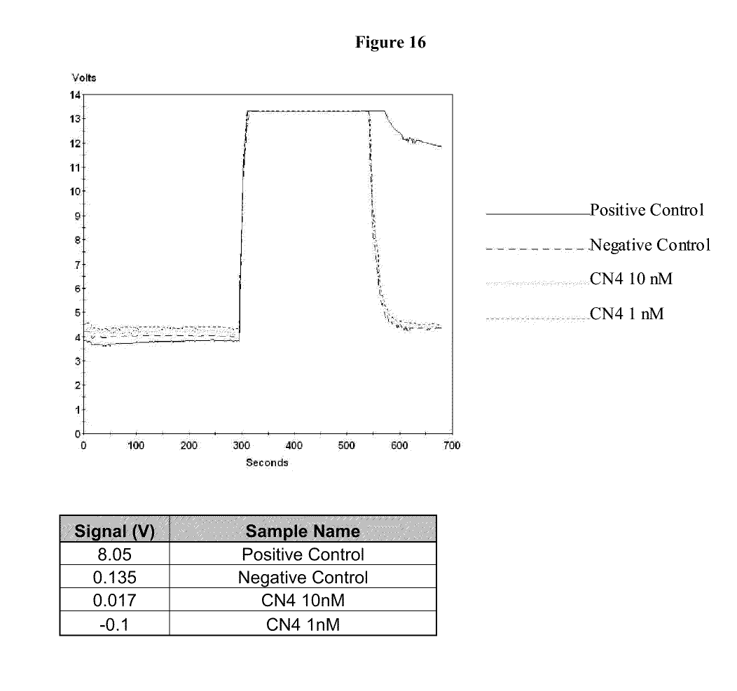

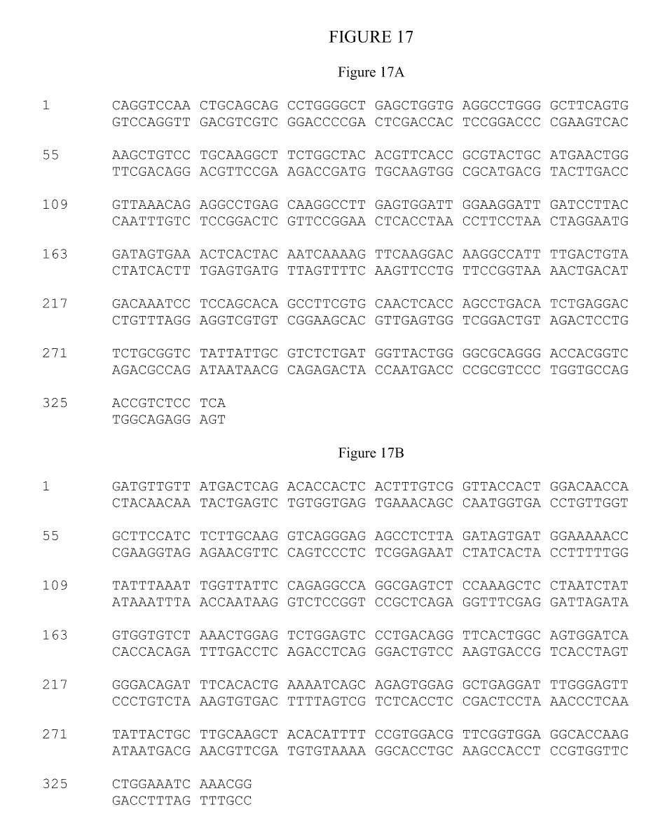

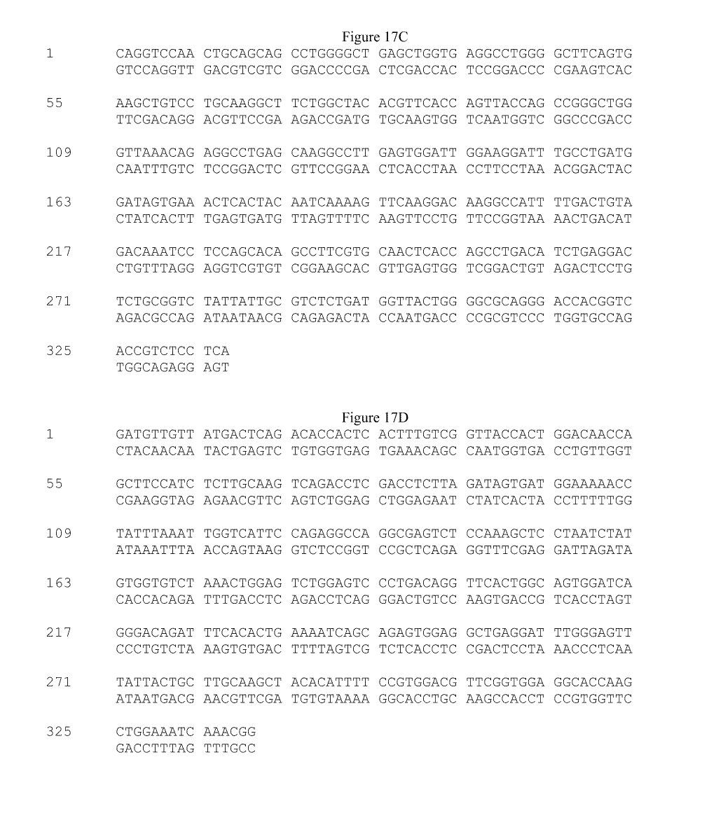

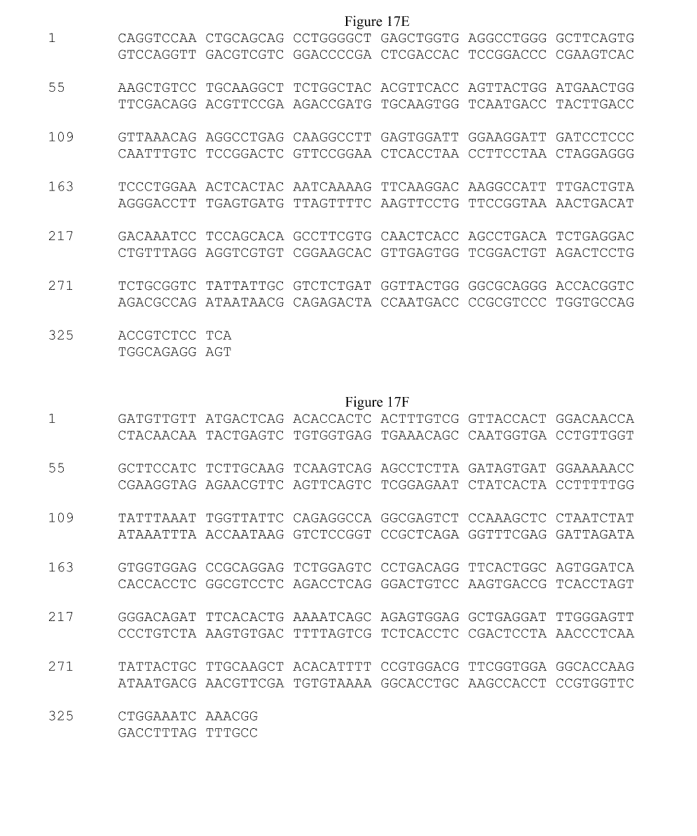

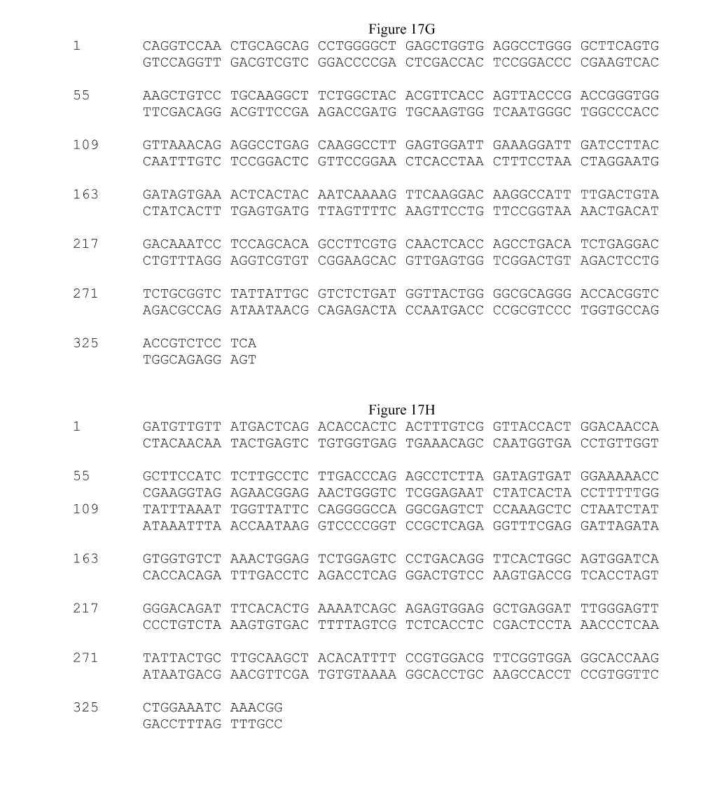

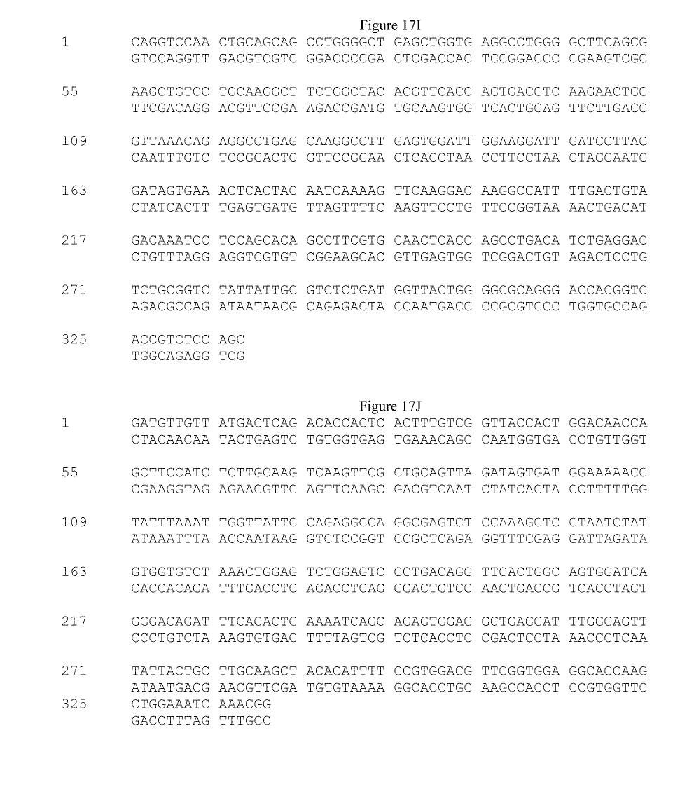

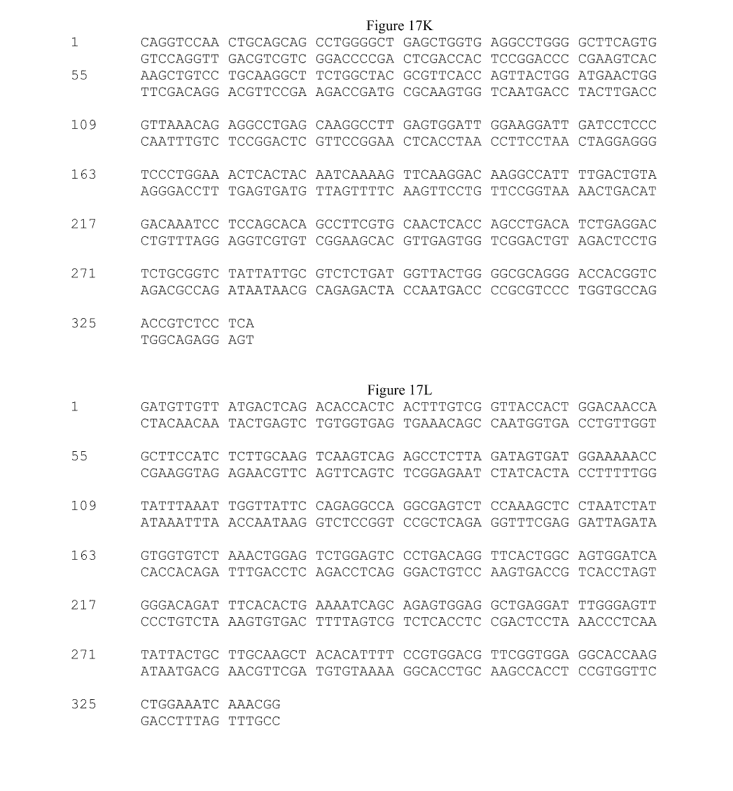

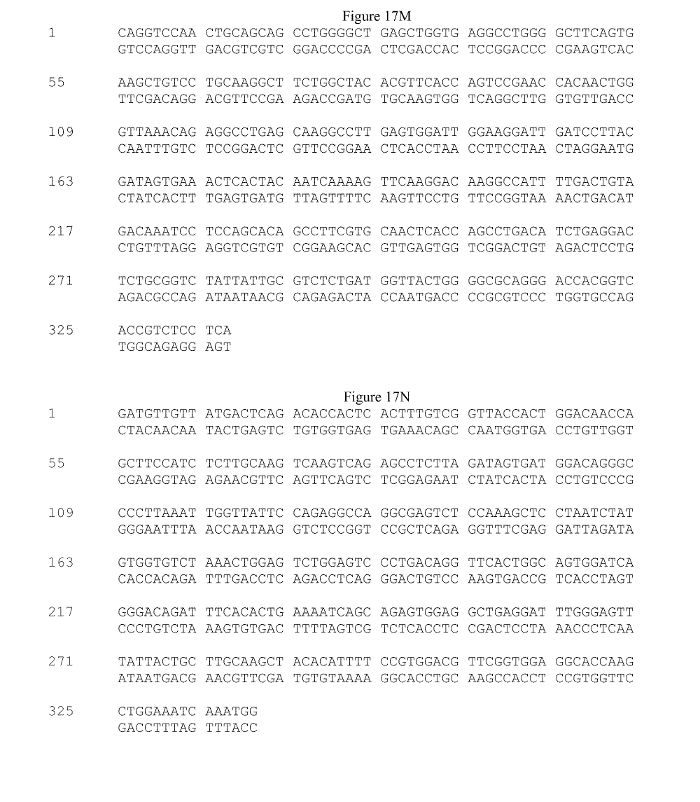

The Kinetic Exclusion Assay® (KinExA®) instrument by Sapidyne Instruments, Inc was used to evaluate negative mimic chimeric mAb binding to human BNP. Azlactone beads and 20-30 ug cBNP(1-32) in carbonate buffer were mixed 2-24 hours at room temperature. Beads were pelleted, supernatant decanted and beads resuspended in Tris-HCl buffer with BSA at 10 mg/ml. Antibody was diluted to 1 or 10 nM final concentration in PBS/1% BSA blocking buffer. On the KinExA® instrument, human BNP coated azlactone beads were collected in a capillary flow cell, washed with blocking buffer and negative mimic antibody or control antibody was passed over the azlactone beads. Finally, the detecting reagent Cy5: F(ab′)2 goat anti-mouse IgG was passed through the beads followed by a blocking buffer wash. The signal generated is reported in volts as seen in FIG. 16. Clone CN4 at both 1 nM and 10 nM mAb concentration does not generate a signal exhibiting its lack of ability to bind to human BNP.

### Example 3

### Stable Cell Line Generation

Chinese Hamster Ovary cell line for human BNP3-631-436CN4CHO was generated for the production of CN4 antibodies. This cell line comprises at least one polynucleotide sequence selected from the group consisting of: SEQ ID NO:82, SEQ ID NO:83, SEQ ID NO:84, SEQ ID NO:85 and combinations thereof.

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the disclosure. It will be readily apparent to one skilled in the art that varying substitutions and modifications may be made to the disclosure disclosed herein without departing from the scope and spirit of the disclosure.

All patents and publications mentioned in the specification are indicative of the levels of those skilled in the art to which the disclosure pertains. All patents and publications are herein incorporated by reference to the same extent as if each individual publication was specifically and individually indicated to be incorporated by reference.

The disclosure illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

What is claimed is:

1. An isolated antibody that does not bind to BNP, wherein said antibody has a variable heavy domain and a variable light domain, the variable heavy domain comprising a heavy chain complementarity determining region (CDR) 1, a heavy chain CDR 2, a heavy chain CDR 3, a heavy framework region 1, a heavy framework region 2, a heavy framework region 3, a heavy framework region 4, the variable light domain comprising a light chain CDR 1, a light chain CDR 2, a light chain CDR 3, a light framework region 1, a light framework region 2, a light framework region 3, and a light framework region 4, wherein said antibody comprises at least one of:
(a) a heavy chain CDR 1 having an amino acid sequence of the formula of: a heavy chain CDR 1 having an amino acid sequence of the formula of:
(SEQ ID NO:1)
Gly-Tyr-Xaa₉-Phe-Thr-Xaa₁₀-Xaa₁₁-Xaa₁₂-Xaa₁₃-Xaa₁₄
wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine, provided that if (a) Xaa₉ is threonine, Xaa₁₀ is serine, Xaa₁₁ is tyrosine, Xaa₁₂ is tryptophan, Xaa₁₃ is methionine then Xaa₁₄ is glycine; (b) Xaa₁₀ is serine, Xaa₁₁ is tyrosine, Xaa₁₂ is tryptophan, Xaa₁₃ is methionine, Xaa₁₄ is alanine, then Xaa₉ is alanine; (c) Xaa₉ is threonine, Xaa₁₁ is tyrosine, Xaa₁₂ is tryptophan, Xaa₁₃ is methionine, Xaa₁₄ is asparagine, then Xaa₁₀ is alanine; (d) Xaa₉ is threonine, Xaa₁₀ is serine, Xaa₁₂ is tryptophan, Xaa₁₃ is methionine, Xaa₁₄ is asparagine, then Xaa₁₁ is aspartic acid or proline; (e) Xaa₉ is threonine, Xaa₁₀ is serine, Xaa₁₁ is tyrosine, Xaa₁₃ is methionine, Xaa₁₄ is asparagine then Xaa₁₂ is cysteine, glutamine, proline, valine or asparagine; or (f) Xaa₉ is threonine, Xaa₁₀ is serine, Xaa₁₁ is tyrosine, Xaa₁₂ is tryptophan, Xaa₁₄ is asparagine then Xaa₁₃ is proline, threonine, lysine or histidine;
(b) a heavy chain CDR 2 having an amino acid sequence of the formula of:
(SEQ ID NO:2)
Arg-Ile-Xaa₁-Xaa₂-Xaa₃-Xaa₄-Xaa₅-Glu-Thr-His-Tyr-
Asn-Gln-Lys-Phe-Lys-Asp
wherein Xaa₁ is aspartic acid or cysteine; Xaa₂ is proline or leucine; Xaa₃ is tyrosine, proline or methionine; Xaa₄ is aspartic acid or serine and Xaa₅ is serine or leucine, provided that if: (a) Xaa₁ is aspartic acid, Xaa₂ is proline, Xaa₃ is tyrosine, Xaa₄ is aspartic acid then Xaa₅ is leucine; (b) Xaa₂ is proline, Xaa₃ is tyrosine, Xaa₄ is aspartic acid, Xaa₅ is serine, then Xaa₁ is cysteine; (c) Xaa₁ is aspartic acid, Xaa₃ is tyrosine, Xaa₄ is aspartic acid, Xaa₅ is serine, then Xaa₂ is leucine; or (d) Xaa₁ is aspartic acid, Xaa₂ is proline, Xaa₄ is aspartic acid, Xaa₅ is serine, then Xaa₃ is proline or methionine; or (e) Xaa₁ is aspartic acid, Xaa₂ is proline, Xaa₃ is tyrosine, Xaa₅ is serine, then Xaa₄ is serine;
(c) a light chain CDR 1 that has an amino acid sequence having a formula of:
(SEQ ID NO:4)
Xaa₁₅-Xaa₁₆-Xaa₁₇-Xaa₁₈-Xaa₁₉-Xaa₂₀-Leu-Asp-Ser-
Asp-Gly-Xaa₂₁-Xaa₂₂-Xaa₂₃-Leu-Asn
wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline, provided that if: (a) Xaa₁₅ is lysine; Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, then Xaa₂₃ is proline; (b) Xaa₁₆ is serine; Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₅ is leucine; (c) Xaa₁₅ is lysine, Xaa₁₇ is serine, Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₆ is valine or leucine; (d) Xaa₁₅ is lysine, Xaa₁₆ is serine; Xaa₁₈ is glutamine, Xaa₁₉ is serine, Xaa₂₀ is leucine, Xaa₂₁ is lysine, Xaa₂₂ is threonine, Xaa₂₃ is tyrosine, then Xaa₁₇ is arginine, aspartic acid or threonine; (e) Xaa₁₅ is lysine, Xaa₁₆ is serine; Xaa₁₇ is serine, Xaa₁₉ is serine, Xaa₂₀ is leucine, Xaa₂₁ is lysine, Xaa₂₂ is threonine, Xaa₂₃ is tyrosine, then Xaa₁₈ is glutamic acid; (f) Xaa₁₅ is lysine, Xaa₁₆ is serine; Xaa₁₇ is serine, Xaa₁₈ is glutamine, Xaa₂₀ is leucine, Xaa₂₁ is lysine, Xaa₂₂ is threonine, Xaa₂₃ is tyrosine, then Xaa₁₉ is aspartic acid or leucine; (g) Xaa₁₅ is lysine, Xaa₁₆ is serine; Xaa₁₇ is serine, Xaa₁₈ is glutamine, Xaa₁₉ is serine, Xaa₂₁ is lysine, Xaa₂₂ is threonine, Xaa₂₃ is tyrosine, then Xaa₂₀ is glutamine; (h) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₂ is threonine, Xaa₂₃ is tyrosine, then Xaa₂₁ is glutamine; or (i) Xaa₁₅ is lysine, Xaa₁₆ is serine; Xaa₁₇ is serine, Xaa₁₈ is glutamine, Xaa₁₉ is serine, Xaa₂₀ is leucine, Xaa₂₁ is lysine, Xaa₂₃ is tyrosine, then Xaa₂₂ is glycine;
(d) a light chain CDR 2 that has an amino acid sequence having a formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine, provided that if (a) Xaa₆ is serine and Xaa₇ is lysine then Xaa₈ is glutamine; (b) Xaa₇ is lysine and Xaa₈ is leucine then Xaa₆ is glutamic acid; or (c) Xaa₆ is serine and Xaa₈ is leucine, then Xaa₇ is proline;
(e) a heavy chain framework 1 that has an amino acid sequence having a formula of:
(SEQ ID NO:114)
Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-Ala-
Ser
wherein Xaa₂₄ is alanine;
(f) a heavy chain framework 2 that has an amino acid sequence having a formula of:
(SEQ ID NO:116)
Trp-Val-Lys-Gln-Arg-Pro-Glu-Gln-Gly-Leu-Glu-Trp-
Ile-Xaa₂₅
wherein Xaa₂₅ glutamic acid;
(g) a light chain framework 2 that has an amino acid sequence having a formula of:
(SEQ ID NO:121)
Trp-Xaa₂₆-Phe-Gln-Xaa27-Pro-Gly-Glu-Ser-Pro-Lys-
Leu-Leu-Ile-Tyr
wherein Xaa₂₆ is leucine or serine and Xaa₂₇ is arginine or glycine, provided that if (a) Xaa₂₆ is leucine then Xaa₂₇ is glycine; or (b) Xaa₂₇ is arginine then Xaa₂₆ is serine;
(h) a light chain framework 4 that has an amino acid sequence having a formula of:
(SEQ ID NO:125)
Trp-Thr-Phe-Gly-Gly-Gly-Thr-Lys-Leu-Glu-Ile-Lys-
Xaa₂₈
wherein Xaa₂₈ is tryptophan; or
(i) any combinations of (a)-(h).

2. The antibody of claim 1, wherein said antibody is a monoclonal antibody, a multispecific antibody, a human antibody, a fully humanized antibody, a partially humanized antibody, an animal antibody, a recombinant antibody, a chimeric antibody, a single-chain Fv, a single chain antibody, a single domain antibody, a Fab fragment, a F(ab′)₂ fragment, a disulfide-linked Fv, a DVD-Ig, a TVD-Ig, an anti-idiotypic antibody, or a functionally active epitope-binding fragment thereof.

3. An isolated antibody that does not bind to BNP, wherein said antibody has a heavy chain complementarity determining region (CDR) 1, a heavy chain CDR 2, a heavy chain CDR 3, a heavy framework region 1, a heavy framework region 2, a heavy framework region 3, a heavy framework region 4, the variable light domain comprising a light chain CDR 1, a light chain CDR 2, a light chain CDR 3, a light framework region 1, a light framework region 2, a light framework region 3, and a light framework region 4, wherein said antibody comprises:
(a) the heavy chain CDR 1 has an amino acid sequence having a formula of:
(SEQ ID NO:1)
Gly-Tyr-Xaa₉-Phe-Thr-Xaa₁₀-Xaa₁₁-Xaa₁₂-Xaa₁₃-Xaa₁₄
wherein Xaa₉ is threonine or alanine; Xaa₁₀ is serine or alanine; Xaa₁₁ is tyrosine, aspartic acid or proline; Xaa₁₂ is tryptophan, cysteine, glutamine, proline, valine or asparagine; Xaa₁₃ is methionine, proline, threonine, lysine or histidine; and Xaa₁₄ is asparagine or glycine;
(b) the heavy chain CDR 2 has an amino acid sequence having a formula of:
(SEQ ID NO:2)
Arg-Ile-Xaa₁-Xaa₂-Xaa₃-Xaa₄-Xaa₅-Glu-Thr-His-Tyr-
Asn-Gln-Lys-Phe-Lys-Asp
wherein Xaa₁ is aspartic acid or cysteine; Xaa₂ is proline or leucine; Xaa₃ is tyrosine, proline or methionine; Xaa₄ is aspartic acid or serine and Xaa₅ is serine or leucine;
(c) the heavy chain CDR 3 has an amino acid sequence of:

| | |
|---|---|
| Asp-Gly-Tyr; | (SEQ ID NO:3) |

(d) the light chain CDR 1 has an amino acid sequence having a formula of:
Xaa₁₅-Xaa₁₆-Xaa₁₇-Xaa₁₈-Xaa₁₉-Xaa₂₀-Leu-Asp-Ser-Asp-Gly-Xaa₂₁-Xaa₂₂-Xaa₂₃-Leu-Asn (SEQ ID NO:4)
wherein Xaa₁₅ is lysine or leucine; Xaa₁₆ is serine, valine or leucine; Xaa₁₇ is serine, arginine, aspartic acid or threonine; Xaa₁₈ is glutamine, glutamic acid, leucine or serine; Xaa₁₉ is serine, aspartic acid, leucine; Xaa₂₀ is leucine or glutamine; Xaa₂₁ is lysine or glutamine; Xaa₂₂ is threonine or glycine; Xaa₂₃ is tyrosine or proline;
(e) the light chain CDR 2 has an amino acid sequence having the formula of:

| | |
|---|---|
| Val-Val-Xaa₆-Xaa₇-Xaa₈-Glu-Ser | (SEQ ID NO:5) |

wherein Xaa₆ is serine or glutamic acid, Xaa₇ is lysine or proline and Xaa₈ is leucine or glutamine;
(f) the light chain CDR 3 has an amino acid sequence having a formula of:

| | |
|---|---|
| Leu-Gln-Ala-Thr-His-Phe-Pro; | (SEQ ID NO:6) |

(g) the heavy chain framework 1 has an amino acid sequence having a formula of:
(SEQ ID NO:130)
Gln-Val-Gln-Leu-Gln-Gln-Pro-Gly-Ala-Glu-Leu-Val-
Arg-Pro-Gly-Ala-Ser-Xaa₂₄-Lys-Leu-Ser-Cys-Lys-Ala-
Ser
wherein Xaa₂₄ is valine or alanine;
(h) the heavy chain framework 2 has an amino acid sequence having a formula of:
(SEQ ID NO:131)
Trp-Val-Lys-Gln-Arg-Pro-Glu-Gln-Gly-Leu-Glu-Trp-
Ile-Xaa₂₅
wherein Xaa₂₅ is glycine or glutamic acid;
(i) the heavy chain framework 3 has an amino acid sequence having a formula of:
(SEQ ID NO:118)
Lys-Ala-Ile-Leu-Thr-Val-Asp-Lys-Ser-Ser-Ser-Thr-
Ala-Phe-Val-Gln-Leu-Thr-Ser-Leu-Thr-Ser-Glu-Asp-
Ser-Ala-Val-Tyr-Tyr-Cys-Val-Ser;
(j) the heavy chain framework 4 has an amino acid sequence having a formula of:
(SEQ ID NO:119)
Trp-Gly-Ala-Gly-Thr-Thr-Cys-Thr-Val-Ser-Ser;
(k) the light chain framework 1 has an amino acid sequence having a formula of:
(SEQ ID NO:120)
Asp-Val-Val-Met-Thr-Gln-Thr-Pro-Leu-Thr-Leu-Ser-
Val-Thr-Thr-Gly-Gln-Pro-Ala-Ser-Ile-Ser-Cys;
(l) the light chain framework 2 has an amino acid sequence having a formula of:
(SEQ ID NO:121)
Trp-Xaa₂₆-Phe-Gln-Xaa₂₇-Pro-Gly-Glu-Ser-Pro-Lys-
Leu-Leu-Ile-Tyr
wherein Xaa₂₆ is leucine or serine;
wherein Xaa₂₇ is arginine or glycine;
(m) the light chain framework 3 has an amino acid sequence having a formula of:
(SEQ ID NO:124)
Gly-Val-Pro-Asp-Arg-Phe-Thr-Gly-Ser-Gly-Ser-Gly-
Thr-Asp-Phe-Thr-leu-Lys-Ile-Ser-Arg-Val-Glu-Ala-
Glu-Asp-leu-Gly-Val-Tyr-Tyr-Cys;
and
(n) the light chain framework 4 has an amino acid sequence having a formula of:
(SEQ ID NO:132)
Trp-Thr-Phe-Gly-Gly-Gly-Thr-Lys-Leu-Glu-Ile-Lys-
Xaa₂₈
wherein Xaa₂₈ is arginine or tryptophan;
provided that each of Xaa₁-Xaa₂₈ are not simultaneously the following: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg.

4. The antibody of claim 3, wherein Xaa₁-Xaa₂₈ are as follows: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ala, Xaa₁₁ is Tyr, Xaa₁₂ is Cys, Xaa₁₃ is Met, Xaa₁₄ is Asn, Xaa₁₅ is Lys, Xaa₁₆ is Val, Xaa₁₇ is Arg, Xaa₁₈ is Glu, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg.

5. The antibody of claim 3, wherein Xaa₁-Xaa₂₈ are as follows: Xaa₁ is Cys, Xaa₂ is Leu, Xaa₃ is Met, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Gln, Xaa₁₃ is Pro, Xaa₁₄ is Gly, Xaa₁₅ is Lys, Xaa₁₆ is Ser, Xaa₁₇ is Asp, Xaa₁₈ is Leu, Xaa₁₉ is Asp, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Ser, Xaa₂₇ is Arg and Xaa₂₈ is Arg.

6. The antibody of claim 3, wherein Xaa₁-Xaa₂₈ are as follows: Xaa₁ is Asp, Xaa₂ is Pro, Xaa₃ is Pro, Xaa₄ is Ser, Xaa₅ is Leu, Xaa₆ is Glu, Xaa₇ is Pro, Xaa₈ is Gln, Xaa₉ is Thr, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg.

7. The antibody of claim 3, wherein Xaa₁-Xaa₂₈ are as follows: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Tyr, Xaa₁₂ is Pro, Xaa₁₃ is Thr, Xaa₁₄ is Gly, Xaa₁₅ is Leu, Xaa₁₆ is Leu, Xaa₁₇ is Thr, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Glu, Xaa₂₆ is Leu, Xaa₂₇ is Gly and Xaa₂₈ is Arg.

8. The antibody of claim 3, wherein Xaa₁-Xaa₂₈ are as follows: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁₁ is Asp, Xaa₁₂ is Val, Xaa₁₃ is Lys, Xaa₁₄ is Asn, Xaa₁₅ is Lys, Xaa₁₆ is Ser, Xaa₁₇ is Ser, Xaa₁₈ is Ser, Xaa₁₉ is Leu, Xaa₂₀ is Gln, Xaa₂₁ is Lys, Xaa₂₂ is Thr, Xaa₂₃ is Tyr, Xaa₂₄ is Ala, Xaa₂₅ is Gly, Xaa₂₆ is Leu, Xaa₂₇ is Arg and Xaa₂₈ is Arg.

9. The antibody of claim 3, wherein Xaa₁-Xaa₂₈ are as follows: Xaa₁ is Asp, Xaa₂ is Pro, Xaa₃ is Pro, Xaa₄ is Ser, Xaa₅ is Leu, Xaa₆ is Ser, Xaa₇ is Lys, Xaa₈ is Leu, Xaa₉ is Ala, Xaa₁₀ is Ser, Xaa₁₁ is Tyr, Xaa₁₂ is Trp, Xaa₁₃ is Met, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Lys, Xaa₂₂ is Thr, Xaa₂₃ is Tyr, Xaa₂₄ is Val, Xaa₂₅ is Gly, Xaa₂₆ is Leu, Xaa₂₇ is Arg and Xaa₂₈ is Arg.

10. The antibody of claim 3, wherein Xaa₁-Xaa₂₈ are as follows: Xaa₁ is Asp, Xaa₂ is Pro, Xaa₃ is Tyr, Xaa₄ is Asp, Xaa₅ is Ser, Xaa₆ is Ser, Xaa₇ is Lys, Xaa₈ is Leu, Xaa₉ is Thr, Xaa₁₀ is Ser, Xaa₁ is Pro, Xaa₁₂ is Asn, Xaa₁₃ is His, Xaa₁₄ is Asn, Xaa₁₅ is Lys, Xaa₁₆ is Ser, Xaa₁₇ is Ser, Xaa₁₈ is Gln, Xaa₁₉ is Ser, Xaa₂₀ is Leu, Xaa₂₁ is Gln, Xaa₂₂ is Gly, Xaa₂₃ is Pro, Xaa₂₄ is Val, Xaa₂₅ is Gly, Xaa₂₆ is Leu, Xaa₂₇ is Arg and Xaa₂₈ is Trp.

11. The antibody of claim 3, wherein:
(a) the heavy chain CDR 1 has an amino acid sequence comprising SEQ ID NO:102, SEQ ID NO:103, SEQ ID NO:104, SEQ ID NO:105, SEQ ID NO:106 or SEQ ID NO:107;
(b) the heavy chain CDR 2 has an amino acid sequence comprising SEQ ID NO:69, SEQ ID NO:108 or SEQ ID NO:129;
(c) the light chain CDR 1 has an amino acid sequence comprising SEQ ID NO:109, SEQ ID NO:110, SEQ ID NO:111, SEQ ID NO:112 or SEQ ID NO:113;
(d) the light chain CDR 2 has an amino acid sequence comprising SEQ ID NO:70;
(e) the heavy chain framework 1 has an amino acid sequence comprising SEQ ID NO:115;
(f) the heavy chain framework 2 has an amino acid sequence comprising SEQ ID NO:117;
(g) the light chain framework 2 has an amino acid sequence comprising SEQ ID NO:122 or SEQ ID NO:123; or
(h) the light chain framework 4 has an amino acid sequence comprising SEQ ID NO:126.

12. The antibody of claim 3, wherein:
(a) the heavy chain CDR 2 has an amino acid sequence comprising SEQ ID NO:69; and
(b) the light chain CDR 2 has an amino acid sequence comprising SEQ ID NO:70.

13. The antibody of claim 3, wherein said antibody is a monoclonal antibody, a multispecific antibody, a human antibody, a fully humanized antibody, a partially humanized antibody, an animal antibody, a recombinant antibody, a chimeric antibody, a single-chain Fv, a single chain antibody, a single domain antibody, a Fab fragment, a F(ab′)₂ fragment, a disulfide-linked Fv, a DVD-Ig, a TVD-Ig, an anti-idiotypic antibody, or a functionally active epitope-binding fragment thereof.

14. An isolated polynucleotide comprising a polynucleotide sequence having a sequence of SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101 and full complements thereof.

15. A heterophilic blocking agent for use in a BNP immunoassay, the agent comprising the antibody of claims 1 or 3.

16. A method for reducing heterophilic interference in a BNP immunoassay, the method comprising the step of:
adding the heterophilic blocking agent of claims 1 or 3 to an immunoassay.

17. An immunoassay for hBNP or hBNP fragment, wherein said immunoassay comprises an antibody of claims 1 or 3.

18. Chinese hamster ovary (“CHO”) cell line BNP3-631-436CN4CHO that expresses the CN4 antibody, wherein said cell line comprises at least one polynucleotide sequence selected from the group consisting of: SEQ ID NO:82, SEQ ID NO:83, SEQ ID NO:84, SEQ ID NO:85 and combinations thereof.

19. An antibody made from DNA extracted from the CHO cell line BNP3-631-436CN4CHO, wherein said DNA comprises at least one polynucleotide sequence selected from the group consisting of: SEQ ID NO:82, SEQ ID NO:83, SEQ ID NO:84, SEQ ID NO:85 and combinations thereof.

20. A chimeric antibody or a fragment thereof produced by CHO cell line BNP3-631-436CN4CHO, wherein said antibody comprises DNA comprising at least one polynucleotide sequence selected from the group consisting of: SEQ ID NO:82, SEQ ID NO:83, SEQ ID NO:84, SEQ ID NO:85 and combinations thereof.

21. A kit for use in an immunoassay for hBNP or hBNP fragment, the kit comprising:
(a) heterophilic blocking agent comprising the antibody of claims 1 or 3; and
(b) instructions for conducting the assay of the test sample.


SEQUENCE LISTING (132 sequences)

SEQ ID NO: 1 - protein, 10 aa, Artificial Sequence
  Description of Artificial Sequence Synthetic Heavy Chain CDR1
GYXFTXXXXX

SEQ ID NO: 2 - protein, 17 aa, Artificial Sequence
  Description of Artificial Sequence Synthetic Heavy Chain CDR2
RIXXXXXETHYNQKFKD

SEQ ID NO: 3 - protein, 3 aa, Artificial Sequence
  Description of Artificial Sequence Synthetic Heavy Chain CDR3
DGY

SEQ ID NO: 4 - protein, 16 aa, Artificial Sequence
  Description of Artificial Sequence Synthetic Light Chain CDR1
XXXXXXLDSDGXXXLN

SEQ ID NO: 5 - protein, 7 aa, Artificial Sequence
  Description of Artificial Sequence Synthetic Light Chain CDR2
VVXXXES

SEQ ID NO: 6 - protein, 7 aa, Artificial Sequence
  Description of Artificial Sequence Synthetic Light Chain CDR3
LQATHFP

SEQ ID NO: 7 - protein, 23 aa, Artificial sequence
  Description of Artificial Sequence Synthetic N-terminal amino acid sequence of the 3-631-436 light chain
DVVMTQTPLTLSVTTGQPASISC

SEQ ID NO: 8 - DNA, 24 bp, Artificial sequence
  Description of Artificial Sequence Synthetic primer
gaygtngtnatgacncaracnccn

SEQ ID NO: 9 - protein, 15 aa, Artificial sequence
  Description of Artificial Sequence Synthetic peptide
GPAKELTPLKEAKVS

SEQ ID NO: 10 - protein, 32 aa, Homo sapiens
SPKMVQGSGCFGRKMDRISSSSGLGCKVLRRH

SEQ ID NO: 11 - DNA, 5700 bp, Artificial sequence
  Description of Artificial Sequence Synthetic vector
acggattagaagccgccgagcgggtgacagccctccgaaggaagactctcctccgtgcgtcctcgtcctcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcattaaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattctacttcatacattttcaattaagatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaagcttctgcaggctagtggtgagaacaaggtggagtacgcgccggcgttgatggccttgtctgctagcatgactggtggacagcaaatgggtcgggatctgtacgacgatgacgataaggtaccaggatccagtgtggtggaattcgcggcccagccggccatggcccaggtccaactgcagcagcctggggctgagctggtgaggcctggggcttcagtgaagctgtcctgcaaggcttctggctacacgttcaccagttactggatgaactgggttaaacagaggcctgagcaaggccttgagtggattggaaggattgatccttacgatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctcaggtcccgccaaggagttgacgcccctgaaggaggcgaaggtctctgatgttgttatgactcagacaccactcactttgtcggttaccactggacaaccagcttccatctcttgcaagtcaagtcagagcctcttagatagtgatggaaaaacctatttaaattggttattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgggcggccgccctcgagtctagagggcccttcgaaggtaagcctatccctaaccctctcctcggtctcgattctacgcgtaccggtcatcatcaccatcaccattgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaatt

SEQ ID NO: 12 - protein, 274 aa, Artificial sequence
  Description of Artificial Sequence Synthetic construct
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSSGPAKELTPLKEAKVSDVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKRAAALESRGPFEGKPIPNPLLGLDSTRTGHHHHHH

SEQ ID NO: 13 - DNA, 840 bp, Artificial sequence
  Description of Artificial Sequence Synthetic construct
ggcccagccggccatggcccaggtccaactgcagcagcctggggctgagctggtgaggcctggggcttcagtgaagctgtcctgcaaggcttctggctacacgttcaccagttactggatgaactgggttaaacagaggcctgagcaaggccttgagtggattggaaggattgatccttacgatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctcaggtcccgccaaggagttgacgcccctgaaggaggcgaaggtctctgatgttgttatgactcagacaccactcactttgtcggttaccactggacaaccagcttccatctcttgcaagtcaagtcagagcctcttagatagtgatggaaaaacctatttaaattggttattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgggcggccgccctcgagtctagagggcccttcgaaggtaagcctatccctaaccctctcctcggtctcgattctacgcgtaccggtcatcatcaccatcacca

SEQ ID NO: 14 - DNA, 102 bp, Artificial Sequence
  Description of Artificial Sequence Synthetic oligonucleotide
cctggggcttcagtgaagctgtcctgcaaggcttctnnsnnsnnsttcaccagttactggatgaactgggttaaacagaggcctgagcaaggccttgagtgg

SEQ ID NO: 15 - DNA, 102 bp, Artificial sequence
  Description of Artificial Sequence Synthetic oligonucleotide
cctggggcttcagtgaagctgtcctgcaaggcttctggcnnsnnsnnsaccagttactggatgaactgggttaaacagaggcctgagcaaggccttgagtgg

SEQ ID NO: 16 - DNA, 102 bp, Artificial sequence
  Description of Artificial Sequence Synthetic oligonucleotide
cctggggcttcagtgaagctgtcctgcaaggcttctggctacnnsnnsnnsagttactggatgaactgggttaaacagaggcctgagcaaggccttgagtgg

SEQ ID NO: 17 - DNA, 102 bp, Artificial sequence
  Description of Artificial Sequence Synthetic oligonucleotide
cctggggcttcagtgaagctgtcctgcaaggcttctggctacacgnnsnnsnnstactggatgaactgggttaaacagaggcctgagcaaggccttgagtgg

SEQ ID NO: 18 - DNA, 102 bp, Artificial sequence
  Description of Artificial Sequence Synthetic oligonucleotide
cctggggcttcagtgaagctgtcctgcaaggcttctggctacacgttcnnsnnsnnstggatgaactgggttaaacagaggcctgagcaaggccttgagtgg

SEQ ID NO: 19 - DNA, 102 bp, Artificial sequence
  Description of Artificial Sequence Synthetic oligonucleotide
cctggggcttcagtgaagctgtcctgcaaggcttctggctacacgttcaccnnsnnsnssatgaactgggttaaacagaggcctgagcaaggccttgagtgg

SEQ ID NO: 20 - DNA, 102 bp, Artificial sequence
  Description of Artificial Sequence Synthetic oligonucleotide
cctggggcttcagtgaagctgtcctgcaaggcttctggctacacgttcaccagtnnsnnsnnsaactgggttaaacagaggcctgagcaaggccttgagtgg

SEQ ID NO: 21 - DNA, 102 bp, Artificial sequence
  Description of Artificial Sequence Synthetic oligonucleotide
cctggggcttcagtgaagctgtcctgcaaggcttctggctacacgttcaccagttacnnsnnsnnstgggttaaacagaggcctgagcaaggccttgagtgg

SEQ ID NO: 22 - DNA, 102 bp, Artificial Sequence
  Description of Artificial Sequence Synthetic oligonucleotide
aaacagaggcctgagcaaggccttgagtggattggannsnnsnnsccttacgatagtgaaactcactacaatcaaaagttcaaggacaaggccattttgact

SEQ ID NO: 23 - DNA, 102 bp, Artificial Sequence
  Description of Artificial Sequence Synthetic oligonucleotide
aaacagaggcctgagcaaggccttgagtggattggaaggnnsnnsnnstacgatagtgaaactcactacaatcaaaagttcaaggacaaggccattttgact

SEQ ID NO: 24 - DNA, 102 bp, Artificial Sequence
  Description of Artificial Sequence Synthetic oligonucleotide
aaacagaggcctgagcaaggccttgagtggattggaaggattnnsnnsnnsgatagtgaaactcactacaatcaaaagttcaaggacaaggccattttgact

SEQ ID NO: 25 - DNA, 102 bp, Artificial Sequence
  Description of Artificial Sequence Synthetic oligonucleotide
aaacagaggcctgagcaaggccttgagtggattggaaggattgatnnsnnsnnsagtgaaactcactacaatcaaaagttcaaggacaaggccattttgact

SEQ ID NO: 26 - DNA, 102 bp, Artificial Sequence
  Description of Artificial Sequence Synthetic oligonucleotide
aaacagaggcctgagcaaggccttgagtggattggaaggattgatcctnnsnnsnnsgaaactcactacaatcaaaagttcaaggacaaggccattttgact

SEQ ID NO: 27 - DNA, 102 bp, Artificial Sequence
  Description of Artificial Sequence Synthetic oligonucleotide
aaacagaggcctgagcaaggccttgagtggattggaaggattgatccttacnnsnnsnnsactcactacaatcaaaagttcaaggacaaggccattttgact

SEQ ID NO: 28 - DNA, 102 bp, Artificial Sequence
  Description of Artificial Sequence Synthetic oligonucleotide
aaacagaggcctgagcaaggccttgagtggattggaaggattgatccttacgatnnsnnsnnscactacaatcaaaagttcaaggacaaggccattttgact

SEQ ID NO: 29 - DNA, 102 bp, Artificial Sequence
  Description of Artificial Sequence Synthetic oligonucleotide
aaacagaggcctgagcaaggccttgagtggattggaaggattgatccttacgatagtnnsnnsnnstacaatcaaaagttcaaggacaaggccattttgact

SEQ ID NO: 30 - DNA, 99 bp, Artificial Sequence
  Description of Artificial Sequence Synthetic oligonucleotide
gagtggattggaaggattgatccttacgatagtgaannsnnsnnsaatcaaaagttcaaggacaaggccattttgactgtagacaaatcctccagcaca

SEQ ID NO: 31 - DNA, 99 bp, Artificial Sequence
  Description of Artificial Sequence Synthetic oligonucleotide
gagtggattggaaggattgatccttacgatagtgaaactnnsnnsnnscaaaagttcaaggacaaggccattttgactgtagacaaatcctccagcaca

SEQ ID NO: 32 - DNA, 99 bp, Artificial Sequence
  Description of Artificial Sequence Synthetic oligonucleotide
gagtggattggaaggattgatccttacgatagtgaaactcacnnsnnsnnsaagttcaaggacaaggccattttgactgtagacaaatcctccagcaca

SEQ ID NO: 33 - DNA, 99 bp, Artificial Sequence
  Description of Artificial Sequence Synthetic oligonucleotide
gagtggattggaaggattgatccttacgatagtgaaactcactacnnsnnsnnsttcaaggacaaggccattttgactgtagacaaatcctccagcaca

SEQ ID NO: 34 - DNA, 99 bp, Artificial Sequence
  Description of Artificial Sequence Synthetic oligonucleotide
gagtggattggaaggattgatccttacgatagtgaaactcactacaatnnsnnsnnsaaggacaaggccattttgactgtagacaaatcctccagcaca

SEQ ID NO: 35 - DNA, 99 bp, Artificial Sequence
  Description of Artificial Sequence Synthetic oligonucleotide
gagtggattggaaggattgatccttacgatagtgaaactcactacaatcaannsnnsnnsgacaaggccattttgactgtagacaaatcctccagcaca

SEQ ID NO: 36 - DNA, 99 bp, Artificial Sequence
  Description of Artificial Sequence Synthetic oligonucleotide
gagtggattggaaggattgatccttacgatagtgaaactcactacaatcaaaagnnsnnsnnsaaggccattttgactgtagacaaatcctccagcaca

SEQ ID NO: 37 - DNA, 93 bp, Artificial Sequence
  Description of Artificial Sequence Synthetic oligonucleotide
agcctgacatctgaggactctgcggtctattattgcgtctctnnsnnsnnstggggcgcagggaccacggtcaccgtctcctcaggtcccgcc

SEQ ID NO: 38 - DNA, 102 bp, Artificial Sequence
  Description of Artificial Sequence Synthetic oligonucleotide
tcggttaccactggacaaccagcttccatctcttgcnnsnnsnnscagagcctcttagatagtgatggaaaaacctatttaaattggttattccagaggcca

SEQ ID NO: 39 - DNA, 102 bp, Artificial Sequence
  Description of Artificial Sequence Synthetic oligonucleotide
tcggttaccactggacaaccagcttccatctcttgcaagnnsnnsnnsagcctcttagatagtgatggaaaaacctatttaaattggttattccagaggcca

SEQ ID NO: 40 - DNA, 102 bp, Artificial Sequence
  Description of Artificial Sequence Synthetic oligonucleotide
tcggttaccactggacaaccagcttccatctcttgcaagtcannsnnsnnsctcttagatagtgatggaaaaacctatttaaattggttattccagaggcca

SEQ ID NO: 41 - DNA, 102 bp, Artificial Sequence
  Description of Artificial Sequence Synthetic oligonucleotide
tcggttaccactggacaaccagcttccatctcttgcaagtcaagtnnsnnsnnsttagatagtgatggaaaaacctatttaaattggttattccagaggcca

SEQ ID NO: 42 - DNA, 102 bp, Artificial Sequence
  Description of Artificial Sequence Synthetic oligonucleotide
tcggttaccactggacaaccagcttccatctcttgcaagtcaagtcagnnsnnsnnsgatagtgatggaaaaacctatttaaattggttattccagaggcca

SEQ ID NO: 43 - DNA, 102 bp, Artificial Sequence
  Description of Artificial Sequence Synthetic oligonucleotide
tcggttaccactggacaaccagcttccatctcttgcaagtcaagtcagagcnnsnnsnnsagtgatggaaaaacctatttaaattggttattccagaggcca

SEQ ID NO: 44 - DNA, 102 bp, Artificial Sequence
  Description of Artificial Sequence Synthetic oligonucleotide
tcggttaccactggacaaccagcttccatctcttgcaagtcaagtcagagcctcnnsnnsnnsgatggaaaaacctatttaaattggttattccagaggcca

SEQ ID NO: 45 - DNA, 102 bp, Artificial Sequence; identical to SEQ ID NO: 44
  Description of Artificial Sequence Synthetic oligonucleotide

SEQ ID NO: 46 - DNA, 96 bp, Artificial Sequence
  Description of Artificial Sequence Synthetic oligonucleotide
tccatctcttgcaagtcaagtcagagcctcttagatnnsnnsnnsaaaacctatttaaattggttattccagaggccaggcgagtctccaaagctc

SEQ ID NO: 47 - DNA, 96 bp, Artificial Sequence
  Description of Artificial Sequence Synthetic oligonucleotide
tccatctcttgcaagtcaagtcagagcctcttagatagtnnsnnsnnsacctatttaaattggttattccagaggccaggcgagtctccaaagctc

SEQ ID NO: 48 - DNA, 96 bp, Artificial Sequence; identical to SEQ ID NO: 47
  Description of Artificial Sequence Synthetic oligonucleotide

SEQ ID NO: 49 - DNA, 96 bp, Artificial Sequence
  Description of Artificial Sequence Synthetic oligonucleotide
tccatctcttgcaagtcaagtcagagcctcttagatagtgatggannsnnsnnsttaaattggttattccagaggccaggcgagtctccaaagctc

SEQ ID NO: 50 - DNA, 96 bp, Artificial Sequence
  Description of Artificial Sequence Synthetic oligonucleotide
tccatctcttgcaagtcaagtcagagcctcttagatagtgatggaaaannsnnsnnsaattggttattccagaggccaggcgagtctccaaagctc

SEQ ID NO: 51 - DNA, 96 bp, Artificial Sequence
  Description of Artificial Sequence Synthetic oligonucleotide
tccatctcttgcaagtcaagtcagagcctcttagatagtgatggaaaaaccnnsnnsnnstggttattccagaggccaggcgagtctccaaagctc

SEQ ID NO: 52 - DNA, 93 bp, Artificial Sequence
  Description of Artificial Sequence Synthetic oligonucleotide
cagaggccaggcgagtctccaaagctcctaatctatnnsnnsnnsaaactggagtctggagtccctgacaggttcactggcagtggatcaggg

SEQ ID NO: 53 - DNA, 93 bp, Artificial Sequence
  Description of Artificial Sequence Synthetic oligonucleotide
cagaggccaggcgagtctccaaagctcctaatctatgtgnnsnnsnnsctggagtctggagtccctgacaggttcactggcagtggatcaggg

SEQ ID NO: 54 - DNA, 93 bp, Artificial Sequence
  Description of Artificial Sequence Synthetic oligonucleotide
cagaggccaggcgagtctccaaagctcctaatctatgtggtgnnsnnsnnsgagtctggagtccctgacaggttcactggcagtggatcaggg

SEQ ID NO: 55 - DNA, 93 bp, Artificial Sequence
  Description of Artificial Sequence Synthetic oligonucleotide
cagaggccaggcgagtctccaaagctcctaatctatgtggtgtctnnsnnsnnstctggagtccctgacaggttcactggcagtggatcaggg

SEQ ID NO: 56 - DNA, 93 bp, Artificial Sequence
  Description of Artificial Sequence Synthetic oligonucleotide
cagaggccaggcgagtctccaaagctcctaatctatgtggtgtctaaannsnnsnnsggagtccctgacaggttcactggcagtggatcaggg

SEQ ID NO: 57 - DNA, 99 bp, Artificial Sequence
  Description of Artificial Sequence Synthetic oligonucleotide
agagtggaggctgaggatttgggagtttattactgcnnsnnsnnsacacattttccgtggacgttcggtggaggcaccaagctggaaatcaaacgggcg

SEQ ID NO: 58 - DNA, 99 bp, Artificial Sequence
  Description of Artificial Sequence Synthetic oligonucleotide
agagtggaggctgaggatttgggagtttattactgcttgnnsnnsnnscattttccgtggacgttcggtggaggcaccaagctggaaatcaaacgggcg

SEQ ID NO: 59 - DNA, 99 bp, Artificial Sequence
  Description of Artificial Sequence Synthetic oligonucleotide
agagtggaggctgaggatttgggagtttattactgcttgcaannsnnsnnstttccgtggacgttcggtggaggcaccaagctggaaatcaaacgggcg

SEQ ID NO: 60 - DNA, 99 bp, Artificial Sequence
  Description of Artificial Sequence Synthetic oligonucleotide
agagtggaggctgaggatttgggagtttattactgcttgcaagctnnsnnsnnsccgtggacgttcggtggaggcaccaagctggaaatcaaacgggcg

SEQ ID NO: 61 - DNA, 99 bp, Artificial Sequence
  Description of Artificial Sequence Synthetic oligonucleotide
agagtggaggctgaggatttgggagtttattactgcttgcaagctacannsnnsnnstggacgttcggtggaggcaccaagctggaaatcaaacgggcg

SEQ ID NO: 62 - DNA, 99 bp, Artificial Sequence
  Description of Artificial Sequence Synthetic oligonucleotide
agagtggaggctgaggatttgggagtttattactgcttgcaagctacacatnnsnnsnnsacgttcggtggaggcaccaagctggaaatcaaacgggcg

SEQ ID NO: 63 - DNA, 99 bp, Artificial Sequence
  Description of Artificial Sequence Synthetic oligonucleotide
agagtggaggctgaggatttgggagtttattactgcttgcaagctacacattttnnsnnsnnsttcggtggaggcaccaagctggaaatcaaacgggcg

SEQ ID NO: 64 - DNA, 20 bp, Artificial Sequence
  Description of Artificial Sequence Synthetic primer
tagcatgactggtggacagc

SEQ ID NO: 65 - DNA, 18 bp, Artificial Sequence
  Description of Artificial Sequence Synthetic primer
cgtagaatcgagaccgag

SEQ ID NO: 66 - protein, 274 aa, Artificial Sequence; identical to SEQ ID NO: 12
  Description of Artificial Sequence Synthetic construct

SEQ ID NO: 67 - protein, 15 aa, Artificial Sequence
  Description of Artificial Sequence Synthetic peptide
KASQSVDYNGDSYLN

SEQ ID NO: 68 - protein, 32 aa, Homo sapiens; identical to SEQ ID NO: 10

SEQ ID NO: 69 - protein, 16 aa, Artificial Sequence
  Description of Artificial Sequence Synthetic peptide
RIDPPSKETHYNQKFK

SEQ ID NO: 70 - protein, 7 aa, Artificial Sequence
  Description of Artificial Sequence Synthetic peptide
VVEPQES

SEQ ID NO: 71 - protein, 17 aa, Artificial Sequence
  Description of Artificial Sequence Synthetic peptide
RIDPYDSETHYNQKFKD

SEQ ID NO: 72 - protein, 7 aa, Artificial Sequence
  Description of Artificial Sequence Synthetic peptide
VVSKLES

SEQ ID NO: 73 - DNA, 841 bp, Artificial Sequence
  Description of Artificial Sequence Synthetic construct
ggcccagccggccatggcccaggtccaactgcagcagcctggggctgagctggtgaggcctggggcttcagtgaagctgtcctgcaaggcttctggctacacgttcaccagttactggatgaactgggttaaacagaggcctgagcaaggccttgagtggattggaaggattgatccttacgatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctcaggtcccgccaaggagttgacgcccctgaaggaggcgaaggtctctgatgttgttatgactcagacaccactcactttgtcggttaccactggacaaccagcttccatctcttgcaagtcaagtcagagcctcttagatagtgatggaaaaacctatttaaattggttattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgggcggccgccctcgagtctagagggcccttcgaaggtaagcctatccctaaccctctcctcggtctcgattctacgcgtaccggtcatcatcaccatcaccat

SEQ ID NO: 74 - DNA, 336 bp, Artificial Sequence
  Description of Artificial Sequence Synthetic polynucleotide
caggtccaactgcagcagcctggggctgagctggtgaggcctggggcttcagtgaagctgtcctgcaaggcttctggctacacgttcaccgcgtactgcatgaactgggttaaacagaggcctgagcaaggccttgagtggattggaaggattgatccttacgatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctca

SEQ ID NO: 75 - DNA, 336 bp, Artificial Sequence
  Description of Artificial Sequence Synthetic polynucleotide
tgaggagacggtgaccgtggtccctgcgccccagtaaccatcagagacgcaataatagaccgcagagtcctcagatgtcaggctggtgagttgcacgaaggctgtgctggaggatttgtctacagtcaaaatggccttgtccttgaacttttgattgtagtgagtttcactatcgtaaggatcaatccttccaatccactcaaggccttgctcaggcctctgtttaacccagttcatgcagtacgcggtgaacgtgtagccagaagccttgcaggacagcttcactgaagccccaggcctcaccagctcagccccaggctgctgcagttggacctg

SEQ ID NO: 76 - DNA, 339 bp, Artificial Sequence
  Description of Artificial Sequence Synthetic polynucleotide
gatgttgttatgactcagacaccactcactttgtcggttaccactggacaaccagcttccatctcttgcaaggtcagggagagcctcttagatagtgatggaaaaacctatttaaattggttattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgg

SEQ ID NO: 77 - DNA, 339 bp, Artificial Sequence
  Description of Artificial Sequence Synthetic polynucleotide
ccgtttgatttccagcttggtgcctccaccgaacgtccacggaaaatgtgtagcttgcaagcagtaataaactcccaaatcctcagcctccactctgctgattttcagtgtgaaatctgtccctgatccactgccagtgaacctgtcagggactccagactccagtttagacaccacatagattaggagctttggagactcgcctggcctctggaataaccaatttaaataggtttttccatcactatctaagaggctctccctgaccttgcaagagatggaagctggttgtccagtggtaaccgacaaagtgagtggtgtctgagtcataacaacatc

SEQ ID NO: 78 - DNA, 336 bp, Artificial Sequence
  Description of Artificial Sequence Synthetic polynucleotide
caggtccaactgcagcagcctggggctgagctggtgaggcctggggcttcagtgaagctgtcctgcaaggcttctggctacacgttcaccagttaccagccgggctgggttaaacagaggcctgagcaaggccttgagtggattggaaggatttgcctgatggatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctca

SEQ ID NO: 79 - DNA, 336 bp, Artificial Sequence
  Description of Artificial Sequence Synthetic polynucleotide
tgaggagacggtgaccgtggtccctgcgccccagtaaccatcagagacgcaataatagaccgcagagtcctcagatgtcaggctggtgagttgcacgaaggctgtgctggaggatttgtctacagtcaaaatggccttgtccttgaacttttgattgtagtgagtttcactatccatcaggcaaatccttccaatccactcaaggccttgctcaggcctctgtttaacccagcccggctggtaactggtgaacgtgtagccagaagccttgcaggacagcttcactgaagccccaggcctcaccagctcagccccaggctgctgcagttggacctg

SEQ ID NO: 80 - DNA, 339 bp, Artificial Sequence
  Description of Artificial Sequence Synthetic polynucleotide
gatgttgttatgactcagacaccactcactttgtcggttaccactggacaaccagcttccatctcttgcaagtcagacctcgacctcttagatagtgatggaaaaacctatttaaattggtcattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgg

SEQ ID NO: 81 - DNA, 339 bp, Artificial Sequence
  Description of Artificial Sequence Synthetic polynucleotide
ccgtttgatttccagcttggtgcctccaccgaacgtccacggaaaatgtgtagcttgcaagcagtaataaactcccaaatcctcagcctccactctgctgattttcagtgtgaaatctgtccctgatccactgccagtgaacctgtcagggactccagactccagtttagacaccacatagattaggagctttggagactcgcctggcctctggaatgaccaatttaaataggtttttccatcactatctaagaggtcgaggtctgacttgcaagagatggaagctggttgtccagtggtaaccgacaaagtgagtggtgtctgagtcataacaacatc

SEQ ID NO: 82 - DNA, 336 bp, Artificial Sequence
  Description of Artificial Sequence Synthetic polynucleotide
caggtccaactgcagcagcctggggctgagctggtgaggcctggggcttcagtgaagctgtcctgcaaggcttctggctacacgttcaccagttactggatgaactgggttaaacagaggcctgagcaaggccttgagtggattggaaggattgatcctccctccctggaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctca

SEQ ID NO: 83 - DNA, 336 bp, Artificial Sequence
  Description of Artificial Sequence Synthetic polynucleotide
tgaggagacggtgaccgtggtccctgcgccccagtaaccatcagagacgcaataatagaccgcagagtcctcagatgtcaggctggtgagttgcacgaaggctgtgctggaggatttgtctacagtcaaaatggccttgtccttgaacttttgattgtagtgagtttccagggagggaggatcaatccttccaatccactcaaggccttgctcaggcctctgtttaacccagttcatccagtaactggtgaacgtgtagccagaagccttgcaggacagcttcactgaagccccaggcctcaccagctcagccccaggctgctgcagttggacctg

SEQ ID NO: 84 - DNA, 339 bp, Artificial Sequence
  Description of Artificial Sequence Synthetic polynucleotide
gatgttgttatgactcagacaccactcactttgtcggttaccactggacaaccagcttccatctcttgcaagtcaagtcagagcctcttagatagtgatggaaaaacctatttaaattggttattccagaggccaggcgagtctccaaagctcctaatctatgtggtggagccgcaggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgg

SEQ ID NO: 85 - DNA, 339 bp, Artificial Sequence
  Description of Artificial Sequence Synthetic polynucleotide
ccgtttgatttccagcttggtgcctccaccgaacgtccacggaaaatgtgtagcttgcaagcagtaataaactcccaaatcctcagcctccactctgctgattttcagtgtgaaatctgtccctgatccactgccagtgaacctgtcagggactccagactcctgcggctccaccacatagattaggagctttggagactcgcctggcctctggaataaccaatttaaataggtttttccatcactatctaagaggctctgacttgacttgcaagagatggaagctggttgtccagtggtaaccgacaaagtgagtggtgtctgagtcataacaacatc

SEQ ID NO: 86 - DNA, 336 bp, Artificial Sequence
  Description of Artificial Sequence Synthetic polynucleotide
caggtccaactgcagcagcctggggctgagctggtgaggcctggggcttcagtgaagctgtcctgcaaggcttctggctacacgttcaccagttacccgaccgggtgggttaaacagaggcctgagcaaggccttgagtggattgaaaggattgatccttacgatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctca

SEQ ID NO: 87 - DNA, 336 bp, Artificial Sequence
  Description of Artificial Sequence Synthetic polynucleotide
tgaggagacggtgaccgtggtccctgcgccccagtaaccatcagagacgcaataatagaccgcagagtcctcagatgtcaggctggtgagttgcacgaaggctgtgctggaggatttgtctacagtcaaaatggccttgtccttgaacttttgattgtagtgagtttcactatcgtaaggatcaatcctttcaatccactcaaggccttgctcaggcctctgtttaacccacccggtcgggtaactggtgaacgtgtagccagaagccttgcaggacagcttcactgaagccccaggcctcaccagctcagccccaggctgctgcagttggacctg

SEQ ID NO: 88 - DNA, 339 bp, Artificial Sequence
  Description of Artificial Sequence Synthetic polynucleotide
gatgttgttatgactcagacaccactcactttgtcggttaccactggacaaccagcttccatctcttgcctcttgacccagagcctcttagatagtgatggaaaaacctatttaaattggttattccaggggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgg

SEQ ID NO: 89 - DNA, 339 bp, Artificial Sequence
  Description of Artificial Sequence Synthetic polynucleotide
ccgtttgatttccagcttggtgcctccaccgaacgtccacggaaaatgtgtagcttgcaagcagtaataaactcccaaatcctcagcctccactctgctgattttcagtgtgaaatctgtccctgatccactgccagtgaacctgtcagggactccagactccagtttagacaccacatagattaggagctttggagactcgcctggcccctggaataaccaatttaaataggtttttccatcactatctaagaggctctgggtcaagaggcaagagatggaagctggttgtccagtggtaaccgacaaagtgagtggtgtctgagtcataacaacatc

SEQ ID NO: 90 - DNA, 336 bp, Artificial Sequence
  Description of Artificial Sequence Synthetic polynucleotide
caggtccaactgcagcagcctggggctgagctggtgaggcctggggcttcagcgaagctgtcctgcaaggcttctggctacacgttcaccagtgacgtcaagaactgggttaaacagaggcctgagcaaggccttgagtggattggaaggattgatccttacgatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctccagc

SEQ ID NO: 91 - DNA, 336 bp, Artificial Sequence
  Description of Artificial Sequence Synthetic polynucleotide
gctggagacggtgaccgtggtccctgcgccccagtaaccatcagagacgcaataatagaccgcagagtcctcagatgtcaggctggtgagttgcacgaaggctgtgctggaggatttgtctacagtcaaaatggccttgtccttgaacttttgattgtagtgagtttcactatcgtaaggatcaatccttccaatccactcaaggccttgctcaggcctctgtttaacccagttcttgacgtcactggtgaacgtgtagccagaagccttgcaggacagcttcgctgaagccccaggcctcaccagctcagccccaggctgctgcagttggacctg

SEQ ID NO: 92 - DNA, 339 bp, Artificial Sequence
  Description of Artificial Sequence Synthetic polynucleotide
gatgttgttatgactcagacaccactcactttgtcggttaccactggacaaccagcttccatctcttgcaagtcaagttcgctgcagttagatagtgatggaaaaacctatttaaattggttattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgg

SEQ ID NO: 93 - DNA, 339 bp, Artificial Sequence
  Description of Artificial Sequence Synthetic polynucleotide
ccgtttgatttccagcttggtgcctccaccgaacgtccacggaaaatgtgtagcttgcaagcagtaataaactcccaaatcctcagcctccactctgctgattttcagtgtgaaatctgtccctgatccactgccagtgaacctgtcagggactccagactccagtttagacaccacatagattaggagctttggagactcgcctggcctctggaataaccaatttaaataggtttttccatcactatctaactgcagcgaacttgacttgcaagagatggaagctggttgtccagtggtaaccgacaaagtgagtggtgtctgagtcataacaacatc

SEQ ID NO: 94 - DNA, 336 bp, Artificial Sequence
  Description of Artificial Sequence Synthetic polynucleotide
caggtccaactgcagcagcctggggctgagctggtgaggcctggggcttcagtgaagctgtcctgcaaggcttctggctacgcgttcaccagttactggatgaactgggttaaacagaggcctgagcaaggccttgagtggattggaaggattgatcctccctccctggaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctca

SEQ ID NO: 95 - DNA, 336 bp, Artificial Sequence
  Description of Artificial Sequence Synthetic polynucleotide
tgaggagacggtgaccgtggtccctgcgccccagtaaccatcagagacgcaataatagaccgcagagtcctcagatgtcaggctggtgagttgcacgaaggctgtgctggaggatttgtctacagtcaaaatggccttgtccttgaacttttgattgtagtgagtttccagggagggaggatcaatccttccaatccactcaaggccttgctcaggcctctgtttaacccagttcatccagtaactggtgaacgcgtagccagaagccttgcaggacagcttcactgaagccccaggcctcaccagctcagccccaggctgctgcagttggacctg

SEQ ID NO: 96 - DNA, 339 bp, Artificial Sequence
  Description of Artificial Sequence Synthetic polynucleotide
gatgttgttatgactcagacaccactcactttgtcggttaccactggacaaccagcttccatctcttgcaagtcaagtcagagcctcttagatagtgatggaaaaacctatttaaattggttattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaacgg

SEQ ID NO: 97 - DNA, 339 bp, Artificial Sequence
  Description of Artificial Sequence Synthetic polynucleotide
ccgtttgatttccagcttggtgcctccaccgaacgtccacggaaaatgtgtagcttgcaagcagtaataaactcccaaatcctcagcctccactctgctgattttcagtgtgaaatctgtccctgatccactgccagtgaacctgtcagggactccagactccagtttagacaccacatagattaggagctttggagactcgcctggcctctggaataaccaatttaaataggtttttccatcactatctaagaggctctgacttgacttgcaagagatggaagctggttgtccagtggtaaccgacaaagtgagtggtgtctgagtcataacaacatc

SEQ ID NO: 98 - DNA, 336 bp, Artificial Sequence
  Description of Artificial Sequence Synthetic polynucleotide
caggtccaactgcagcagcctggggctgagctggtgaggcctggggcttcagtgaagctgtcctgcaaggcttctggctacacgttcaccagtccgaaccacaactgggttaaacagaggcctgagcaaggccttgagtggattggaaggattgatccttacgatagtgaaactcactacaatcaaaagttcaaggacaaggccattttgactgtagacaaatcctccagcacagccttcgtgcaactcaccagcctgacatctgaggactctgcggtctattattgcgtctctgatggttactggggcgcagggaccacggtcaccgtctcctca

SEQ ID NO: 99 - DNA, 336 bp, Artificial Sequence
  Description of Artificial Sequence Synthetic polynucleotide
tgaggagacggtgaccgtggtccctgcgccccagtaaccatcagagacgcaataatagaccgcagagtcctcagatgtcaggctggtgagttgcacgaaggctgtgctggaggatttgtctacagtcaaaatggccttgtccttgaacttttgattgtagtgagtttcactatcgtaaggatcaatccttccaatccactcaaggccttgctcaggcctctgtttaacccagttgtggttcggactggtgaacgtgtagccagaagccttgcaggacagcttcactgaagccccaggcctcaccagctcagccccaggctgctgcagttggacctg

SEQ ID NO: 100 - DNA, 339 bp, Artificial Sequence
  Description of Artificial Sequence Synthetic polynucleotide
gatgttgttatgactcagacaccactcactttgtcggttaccactggacaaccagcttccatctcttgcaagtcaagtcagagcctcttagatagtgatggacagggccccttaaattggttattccagaggccaggcgagtctccaaagctcctaatctatgtggtgtctaaactggagtctggagtccctgacaggttcactggcagtggatcagggacagatttcacactgaaaatcagcagagtggaggctgaggatttgggagtttattactgcttgcaagctacacattttccgtggacgttcggtggaggcaccaagctggaaatcaaatgg

SEQ ID NO: 101 - DNA, 339 bp, Artificial Sequence
  Description of Artificial Sequence Synthetic polynucleotide
ccatttgatttccagcttggtgcctccaccgaacgtccacggaaaatgtgtagcttgcaagcagtaataaactcccaaatcctcagcctccactctgctgattttcagtgtgaaatctgtccctgatccactgccagtgaacctgtcagggactccagactccagtttagacaccacatagattaggagctttggagactcgcctggcctctggaataaccaatttaaggggccctgtccatcactatctaagaggctctgacttgacttgcaagagatggaagctggttgtccagtggtaaccgacaaagtgagtggtgtctgagtcataacaacatc

SEQ ID NO: 102 - protein, 10 aa, Artificial Sequence
  Description of Artificial Sequence Synthetic peptide
GYTFTAYCMN

SEQ ID NO: 103 - protein, 10 aa, Artificial Sequence
  Description of Artificial Sequence Synthetic peptide
GYTFTSYQPG

SEQ ID NO: 104 - protein, 10 aa, Artificial Sequence
  Description of Artificial Sequence Synthetic peptide
GYTFTSYPTG

SEQ ID NO: 105 - protein, 10 aa, Artificial Sequence
  Description of Artificial Sequence Synthetic peptide
GYTFTSDVKN

SEQ ID NO: 106 - protein, 10 aa, Artificial Sequence
  Description of Artificial Sequence Synthetic peptide
GYAFTSYWMN

SEQ ID NO: 107 - protein, 10 aa, Artificial Sequence
  Description of Artificial Sequence Synthetic peptide
GYTFTSPNHN

SEQ ID NO: 108 - protein, 18 aa, Artificial Sequence
  Description of Artificial Sequence Synthetic peptide
RICLMYDSETHYNQKFKD

SEQ ID NO: 109 - protein, 16 aa, Artificial Sequence
  Description of Artificial Sequence Synthetic peptide
KVRESLLDSDGKTYLN

SEQ ID NO: 110 - protein, 16 aa, Artificial Sequence
  Description of Artificial Sequence Synthetic peptide
KSDLDLLDSDGKTYLN

SEQ ID NO: 111 - protein, 16 aa, Artificial Sequence
  Description of Artificial Sequence Synthetic peptide
LLTQSLLDSDGKTYLN

SEQ ID NO: 112 - protein, 16 aa, Artificial Sequence
  Description of Artificial Sequence Synthetic peptide
KSSSLQLDSDGKTYLN

SEQ ID NO: 113 - protein, 16 aa, Artificial Sequence
  Description of Artificial Sequence Synthetic peptide
KSSQSLLDSDGQGPLN

SEQ ID NO: 114 - protein, 25 aa, Artificial Sequence
  Description of Artificial Sequence Synthetic peptide
QVQLQQPGAELVRPGASAKLSCKAS

SEQ ID NO: 115 - protein, 25 aa, Artificial Sequence
  Description of Artificial Sequence Synthetic peptide
QVQLQQPGAELVRPGASAKLSCKAS

SEQ ID NO: 116 - protein, 14 aa, Artificial Sequence
  Description of Artificial Sequence Synthetic peptide
WVKQRPEQGLEWIE

SEQ ID NO: 117 - protein, 14 aa, Artificial Sequence
  Description of Artificial Sequence Synthetic peptide
WVKQRPEQGLEWIE

SEQ ID NO: 118 - protein, 32 aa, Artificial Sequence
  Description of Artificial Sequence Synthetic peptide
KAILTVDKSSSTAFVQLTSLTSEDSAVYYCVS

SEQ ID NO: 119 - protein, 11 aa, Artificial Sequence
  Description of Artificial Sequence Synthetic peptide
WGAGTTCTVSS

SEQ ID NO: 120 - protein, 23 aa, Artificial Sequence
  Description of Artificial Sequence Synthetic peptide
DVVMTQTPLTLSVTTGQPASISC

SEQ ID NO: 121 - protein, 15 aa, Artificial Sequence
  Description of Artificial Sequence Synthetic peptide
WXFQXPGESPKLLIY

SEQ ID NO: 122 - protein, 15 aa, Artificial Sequence
  Description of Artificial Sequence Synthetic peptide
WSFQRPGESPKLLIY

SEQ ID NO: 123 - protein, 15 aa, Artificial Sequence
  Description of Artificial Sequence Synthetic peptide
WLFQGPGESPKLLIY

SEQ ID NO: 124 - protein, 32 aa, Artificial Sequence
  Description of Artificial Sequence Synthetic peptide
GVPDRFTGSGSGTDFTLKISRVEAEDLGVYYC

SEQ ID NO: 125 - protein, 13 aa, Artificial Sequence
  Description of Artificial Sequence Synthetic peptide
WTFGGGTKLEIKW

SEQ ID NO: 126 - protein, 13 aa, Artificial Sequence
  Description of Artificial Sequence Synthetic peptide
WTFGGGTKLEIKW

SEQ ID NO: 127 - protein, 112 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTCTVSS

SEQ ID NO: 128 - protein, 113 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 129 - protein, 17 aa, Artificial Sequence
  Description of Artificial Sequence Synthetic peptide
RIDPPSLETHYNQKFKD

SEQ ID NO: 130 - protein, 25 aa, Artificial Sequence
  Description of Artificial Sequence Synthetic peptide
QVQLQQPGAELVRPGASXKLSCKAS

SEQ ID NO: 131 - protein, 14 aa, Artificial Sequence
  Description of Artificial Sequence Synthetic peptide
WVKQRPEQGLEWIX

SEQ ID NO: 132 - protein, 13 aa, Artificial Sequence
  Description of Artificial Sequence Synthetic peptide
WTFGGGTKLEIKX